(19) United States
(12) Patent Application Publication
(10) Pub. No.: US 2009/0143761 A1
(43) Pub. Date: Jun. 4, 2009

(54) AGENT DELIVERY SYSTEM AND USES OF SAME

(75) Inventors: Hal C. Cantor, West Bloomfield, MI (US); Scott A. Cantor, West Bloomfield, MI (US); Kenneth H. Swartz, Brighton, MI (US); Robert Hower, Farmington Hills, MI (US)

Correspondence Address: BROOKS KUSHMAN P.C., 1000 TOWN CENTER, TWENTY-SECOND FLOOR, SOUTHFIELD, MI, 48075, US

(73) Assignee: TRANSDERMAL PATENTS COMPANY, LLC, Troy, MI (US)

(21) Appl. No.: 11/949,718

(22) Filed: Dec. 3, 2007

Related U.S. Application Data
(63) Continuation-in-part of application No. PCT/US2006/021761, filed on Jun. 5, 2006.
Application No. PCT/US2006/021761 is a continuation-in-part of application No. PCT/US2006/021762, filed on Jun. 5, 2006.
Application No. PCT/US2006/021762 is a continuation-in-part of application No. PCT/US2006/021763, filed on Jun. 5, 2006.
(60) Provisional application No. 60/687,262, filed on Jun. 3, 2005.
(60) Provisional application No. 60/687,262, filed on Jun. 3, 2005.
(60) Provisional application No. 60/687,262, filed on Jun. 3, 2005.

(51) Int. Cl. A61N 1/30; A61K 38/22; A61K 31/465

(52) U.S. Cl. 604/501; 514/12; 514/343

(57) ABSTRACT

The use of an automated, controllable, and affixable pulsatile for treating diseases, having an automated controller for controlling the delivery of drug to a patient, an agent delivery reservoir containing an agent operatively connected to the automated controller, a reservoir controller operatively connected to the automated controller and the reservoir for controlling the delivery of agent to a patient, and a feedback control operatively connected to the automated controller for providing feedback with regard to the drug requirements of the patient for use in treating diseases.

### CROSS REFERENCE TO RELATED APPLICATIONS

This application is a continuation-in-part of International Patent Application Nos. PCT/US2006/021761, filed 5 Jun. 2006, published in English, which claims the benefit of provisional patent application Ser. No. 60/687,262, filed Jun. 3, 2005; PCT/US2006/021762, filed 5 Jun. 2006, published in English which claims the benefit of provisional patent application Ser. No. 60/687,262, filed Jun. 3, 2005; and PCT/US2006/021763, filed 5 Jun. 2006; and which claims the benefit of provisional patent application Ser. No. 60/687,262, filed Jun. 3, 2005. The disclosures of these applications are hereby incorporated by reference in their entireties.

### BACKGROUND OF THE INVENTION

1. Field of the Invention

Generally, the present invention provides an agent delivery system for use in treating disease. More specifically, the present invention provides an automated system for delivery of drugs or compounds for the treatment of disease.

2. Description of the Related Art

The skin functions as the primary barrier to the transdermal penetration of materials into the body and represents the body's major resistance to the transdermal delivery of beneficial agents such as drugs. To date, efforts have concentrated on reducing the physical resistance of the skin or enhancing the permeability of the skin to facilitate the delivery of drugs by passive diffusion. Various methods of increasing the rate of transdermal drug flux have been attempted, most notably by using chemical flux enhancers.

The delivery of drugs through the skin provides many advantages. Primarily, such a means of delivery is a comfortable, convenient and noninvasive way of administering drugs. The variable rates of absorption and metabolism encountered in oral treatment are avoided, and other inherent inconveniences, e.g., gastrointestinal irritation and the like are eliminated as well. Transdermal drug delivery also makes possible a high degree of control over blood concentrations of any particular drug.

However, many drugs are not suitable for passive transdermal drug delivery because of their size, ionic charge characteristics and hydrophilicity. One method of achieving transdermal administration of such drugs is the use of electrical current to actively transport drugs into the body through intact skin. The method of the present invention relates to such iontophoresis, which is an example of such an administration technique.

Herein the terms “electrotransport”, “iontophoresis”, and “iontophoretic” are used to refer to the delivery of pharmaceutically active agents through a body surface by means of an applied electromotive force to an agent-containing reservoir. The agent may be delivered by electromigration, electroporation, electroosmosis or any combination thereof. Electroosmosis has also been referred to as electrohydrokinesis, electro-convection, and electrically induced osmosis. In general, electroosmosis of a species into a tissue results from the migration of solvent in which the species is contained, as a result of the application of electromotive force to the therapeutic species reservoir, which results in solvent flow induced by electromigration of other ionic species. During the electrotransport process, certain modifications or alterations of the skin may occur such as the formation of transiently existing pores in the skin, also referred to as “electroporation”. Any electrically assisted transport of species enhanced by modifications or alterations of the body surface (e.g., formation of pores in the skin) are also included in the term “electrotransport” as used herein. Thus, as used herein, the terms “electrotransport”, “iontophoresis” and “iontophoretic” refer to (a) the delivery of charged drugs or agents by electromigration, (b) the delivery of uncharged drugs or agents by the process of electroosmosis, (c) the delivery of charged or uncharged drugs by electroporation, (d) the delivery of charged drugs or agents by the combined processes of electromigration and electroosmosis, and/or (e) the delivery of a mixture of charged and uncharged drugs or agents by the combined processes of electromigration and electroosmosis.

Systems for delivering ionized drugs through the skin have been known for some time. British Patent Specification No. 410,009 (1934) describes an iontophoretic delivery device that overcame one of the disadvantages of the early devices, namely, the need to immobilize the patient near a source of electric current. The device was made by forming, from the electrodes and the material containing the drug to be delivered, a galvanic cell which itself produced the current necessary for iontophoretic delivery. This device allowed the patient to move around during drug delivery and thus required substantially less interference with the patient's daily activities than previous iontophoretic delivery systems.

In present day electrotransport devices, at least two electrodes are used simultaneously. Both of these electrodes are disposed so as to be in intimate electrical contact with some portion of the skin of the body. One electrode, called the active or donor electrode, is the electrode from which the drug is delivered into the body. The other electrode, called the counter or return electrode, serves to close the electrical circuit through the body. In conjunction with the patient's skin, the circuit is completed by connection of the electrodes to a source of electrical energy, e.g., a battery, and usually to circuitry capable of controlling current passing through the device. If the ionic substance to be driven into the body is positively charged, then the positive electrode (the anode) can be the active electrode and the negative electrode (the cathode) serves as the counter electrode, completing the circuit. If the ionic substance to be delivered is negatively charged, then the cathodic electrode can be the active electrode and the anodic electrode can be the counter electrode.

All electrotransport agent delivery devices utilize an electrical circuit to electrically connect the power source (e.g., a battery) and the electrodes. In very simple devices such as those disclosed by Ariura et al in U.S. Pat. No. 4,474,570, the “circuit” is merely an electrically conductive wire used to connect the battery to an electrode. Other devices use a variety of electrical components to control the amplitude, polarity, timing, waveform shape, etc. of the electric current supplied by the power source. See, for example, U.S. Pat. No. 5,047,007 issued to McNichols et al.

Existing electrotransport devices additionally require a reservoir or source of the pharmaceutically active agent that is to be delivered or introduced into the body. Such drug reservoirs are connected to an electrode, i.e., an anode or a cathode, of the electrotransport device to provide a fixed or renewable source of one or more desired species or agents. A reservoir would include a reservoir matrix or gel that contains the agent and a reservoir housing which physically contains the reservoir matrix or gel. In addition to the drug reservoir, an electrolyte-containing counter reservoir is generally placed between the counter electrode and the body surface. Typically, the electrolyte within the counter reservoir is a buffered saline solution and does not contain a therapeutic agent. In early electrotransport devices, the donor and counter reservoirs were made of materials such as paper (e.g., filter paper), cotton wadding, fabrics and/or sponges that could easily absorb the drug-containing and electrolyte-containing solutions. In more recent years however the use of such reservoir matrix materials has given way to the use of hydrogels composed of natural or synthetic hydrophilic polymers. See for example, U.S. Pat. No. 4,383,529, to Webster, and U.S. Pat. No. 6,039,977, to Venkatraman. Such hydrophilic polymeric reservoirs are preferred from a number of standpoints, including the ease with which they can be manufactured, the uniform properties and characteristics of synthetic hydrophilic polymers, their ability to quickly absorb aqueous drug and electrolyte solutions, and the ease with which these materials can be handled during manufacturing. Such gel materials can be manufactured to have a solid, non-flowable characteristic. Thus, the reservoirs can be manufactured having a predetermined size and geometry.

Generally, the geometry of a reservoir can be described in terms of three parameters: (1) the average cross-sectional area of the reservoir (“A_{RES}”), defined as the arithmetic mean of reservoir cross-sectional areas measured at a number of different distances from and parallel to the body surface; (2) the average thickness of the reservoir; and (3) the body surface contact area (“A_{BODY}”). References to reservoir housing configuration and the above parameters include not only the parameters of the physical reservoir housing, but also include the physical parameters of the reservoir gel or matrix as well.

Electrotransport drug delivery devices having a reusable controller for use with more than one drug-containing unit have been described. The drug-containing unit can be disconnected from the controller when the drug becomes depleted and a fresh drug-containing unit can then be connected to the controller. The drug-containing unit includes the reservoir housing, the reservoir matrix, and associated physical and electrical elements that enable the unit to be removably connected, both mechanically and electrically to the controller. In this way, the relatively more expensive hardware components of the device (e.g., the batteries, the light-emitting diodes, the circuit hardware, etc.) can be contained in the reusable controller. The relatively less expensive donor reservoir and counter reservoir may be contained in the single use, disposable drug containing unit. See, U.S. Pat. No. 5,320,597, to Sage et al.; U.S. Pat. Nos. 5,358,483 and 5,135,479, both to Sibalis. Electrotransport devices having a reusable electronic controller with single use/disposable drug units have also been proposed for electrotransport systems comprised of a single controller adapted to be used with a plurality of different disposable drug units. For example, WO 96/38198, to Johnson et al., discloses the use of such reusable electrotransport controllers which can be connected to drug units for delivering the same drug, but at different dosing levels, (e.g., a high dose drug unit and a low dose drug unit) which can be connected to the same electrotransport controller. Although these systems go far in reducing the overall cost of transdermal electrotransport drug delivery, further cost reductions are needed in order to make this mode of drug delivery more competitive with traditional delivery methods such as by disposable syringe.

To date, commercial transdermal iontophoretic drug delivery devices (e.g., the Phoresor, sold by Iomed, Inc. of Salt Lake City, Utah; the Dupel Iontophoresis System sold by Empi, Inc. of St. Paul, Minn.; the Webster Sweat Inducer, model 3600, sold by Wescor, Inc. of Logan, Utah) have generally utilized a desk-top electrical power supply unit and a pair of skin contacting electrodes. The donor electrode contains a drug solution while the counter electrode contains a solution of a biocompatible electrolyte salt. The “satellite” electrodes are connected to the electrical power supply unit by long (e.g., 1 2 meters) electrically conductive wires or cables. Examples of desktop electrical power supply units which use “satellite” electrode assemblies are disclosed in Jacobsen et al; U.S. Pat. No. 4,141,359; U.S. Pat. No. 5,006,108, to LaPrade et al; and U.S. Pat. No. 5,254,081, to Maurer.

More recently, small self-contained electrotransport delivery devices adapted to be worn on the skin, sometimes unobtrusively under clothing, for extended periods of time have been proposed. The electrical components in such miniaturized iontophoretic drug delivery devices are also preferably miniaturized, and may be in the form of either integrated circuits (i.e., microchips) or small printed circuits. Electronic components, such as batteries, resistors, pulse generators, capacitors, etc. are electrically connected to form an electronic circuit that controls the amplitude, polarity, timing waveform shape, etc. of the electric current supplied by the power source. Such small self-contained electrotransport delivery devices are disclosed for example in Tapper U.S. Pat. No. 5,224,927; Haak et al; U.S. Pat. No. 5,203,768; Sibalis et al U.S. Pat. No. 5,224,928; and Haynes et al U.S. Pat. No. 5,246,418. One concern, particularly with small self-contained electrotransport delivery devices that are manufactured with the drug to be delivered already in them, is the potential loss in efficacy after a long period of device storage. In an electrotransport device using batteries and other electronic components, all of the components have various shelf lives. If it is known, for example, that the batteries used to power these small delivery devices gradually degrade, and the drug delivery rate may go off specification. It would be advantageous to have a means to limit the active life of the delivery device for a certain period of time (e.g., months) after device manufacture in order to prevent this potential loss in device efficacy.

Application of therapeutic drugs, whether by electrotransport or more traditional (e.g., oral) dosing, can sometimes cause unwanted reactions in certain patients. These reactions can take many forms, including change in heart rate, change in body temperature, sweating, shaking and the like. It would be advantageous to automatically and permanently disable an electrotransport drug delivery device upon encountering such “unwanted” reactions.

The potential for abuse by either oral or parenteral routes of narcotic and other psychoactive drugs is well known. For example, the potential for abuse of the synthetic narcotic drug fentanyl is so high that it has become a major cause of death for anesthesiologists and other hospital workers having access to the drug. In order to prevent abuse of these substances, it has been proposed to provide dosage forms that combine the abusable substance with an amount of an antagonist for the abusable substance sufficient to eliminate the “high” associated with abuse of the substance without eliminating the other therapeutic benefits. See, for example, U.S. Pat. Nos. 4,457,933; 3,493,657; and 3,773,955, all of which are incorporated herein by reference.

Many abusable substances are capable of being administered to the body by direct application of the drug to the skin or mucosa, i.e., nasal, vaginal, oral, or rectal mucosa. See for example U.S. Pat. No. 4,588,580, to Gale et al. They can also be delivered to the body by electrotransport. See U.S. Pat. No. 5,232,438, to Theeuwes et al., which is incorporated herein by reference. Electrotransport devices that are intended to deliver an abusable drug, such as a narcotic analgesic pain-killing drug, could be subject to abuse. It would therefore be useful to develop a device to either limit the ability to abuse or to limit the dependency on the drug.

Additionally, people with a variety of diseases would also benefit from the ability to administer drugs via electrotransport. For example, diabetes is the sixth leading cause of death from disease in the U.S., afflicting an estimated 16 million people. Unfortunately, only slightly more than 10 million are diagnosed. Type 1 diabetes accounts for approximately 5-10% of the cases of diabetes. It is estimated that there is an incidence of 30,000 new cases per year. Most new cases of Type 1 diabetes are presented in patients under the age of 25 years.

Treatment of diabetes and its devastating complications results in significant health care expenditures. Currently, it is estimated that more than 10% of all health-care dollars and about 25% of Medicare dollars are expended on patients with diabetes. At present, there are no methods to prevent or cure diabetes. Type 1 diabetes (formerly known as insulin-dependent diabetes mellitus) affects an estimated 500,000 to 750,000 Americans and is more common among children and young adults.

Diabetes significantly diminishes the quality and shortens the longevity of life. Generally half of all Type 1 diabetics die before reaching the age of 50 years. Diabetes is the leading cause of kidney failure, blindness, and non-traumatic amputations in adults. Other major risk factors include; oral infections, tooth loss, heart disease, stroke, and premature death. Treatment of diabetes and its devastating complications results in significant health care expenditures. Currently, it is estimated that more than 10% of all health-care dollars and about 25% of Medicare dollars are expended on patients with diabetes.

The two most common forms of this disease are referred to as Type 1 diabetes and Type 2 diabetes. This research and development project is aimed at patients suffering from Type 1 diabetes, the form of the disease specifically addressed in the Balanced Budget Act of 1997.

The cause of Type I diabetes is due to the destruction of the insulin-producing (beta) cells in the islets of the pancreas by the body's own immune defense system, hence, an “autoimmune” disease process. The destruction of the islet cells leads to a deficiency of insulin secreted by the pancreas, thereby removing the body's ability to regulate glucose metabolism. The end stage of a patient with type II diabetes is type I diabetes because of the destruction of the function of the pancreas by overstimulation in time.

The only treatment available for these individuals includes daily monitoring of blood glucose (via finger prick blood sampling at multiple times each day) followed by injections or infusions of insulin in the effort to maintain blood glucose levels near the normal range. Since the discovery of insulin in the 1920's, insulin replacement has served as the cornerstone of treatment for Type 1 diabetics. Under conventional therapy, insulin replacement is provided via subcutaneous injections of insulin once or twice each day. For most patients, this treatment by subcutaneous injections involves some combination of short acting regular insulin and other longer acting insulin preparations. This presentation of insulin types is non-physiological, both temporally as well as compositionally, leading to the aforementioned long-term medical complications. This process has been termed “intensive therapy” for diabetes management, and appears to offer the greatest hope of preventing diabetic complications by achieving tight control of the normal blood glucose range.

There has been extended research for the development of subcutaneous glucose sensors for the diabetic patient. The difficulty with these devices is that chemical sensors require periodic calibration in order to ensure accuracy and precision over the duration of operation. In the case of failure, invasive techniques are necessary to replace a subcutaneous device.

Therefore, there is a need for a near-continuous non-invasive device for monitoring composition levels with automated, near-continuous infusion of appropriate amounts of an appropriate compound in the effort to achieve normal, i.e. non-diseased, states at all times. It would be desirable to have such devices available in a condition in which the abuse potential of the device is reduced without diminishing the intended therapeutic efficacy of the device or the abusable substance to be administered.

### SUMMARY OF THE INVENTION

The present invention is a pulsatile agent delivery system is a portable iontophoretic device to be attached to the skin. The device is based upon the micro-electro-mechanical system (MEMS) and/or complementary metal oxide semiconductor (CMOS) technology. The device contains two battery-powered electrodes, which send a charged ion across the skin iontophoretically. The battery can be one or more thin film or watch batteries. The battery can be built into the agent delivery system housing or may be integrated into the detachable agent delivery reservoir. This device can also be used in a hospital setting operating on an AC/DC power source. The agent delivery system of the present invention is controlled by an automated controller, which is based on an integrated circuit, which controls the timing and activation of the iontophoretic delivery of the agent from the agent delivery reservoir. Data from the agent delivery system can be stored and transmitted to an external computer.

The agent delivery system can be configured to both deliver a therapeutic agent and extract interstitial fluid to analyze agent concentration in the body or monitor a surrogate marker to determine when additional agent is necessary. The device unlike other iontophoretic devices is able to deliver the charge on a pulsed basis rather than continuously. The pulsed delivery may be timed to: optimize drug concentration requirements; reduce drug waste; reduce the potential for antibiotic drug resistance; and, developing a tolerance to therapeutic agents. The agent delivery system can vary the pulse to increase the interval between doses or reduce the amount of agent delivered over time. The “ramp down” characteristic is a novel way to wean a patient off an addictive drug.

The pulsatile agent delivery system is used in the following applications:

Category 1: The treatment of diseases by delivering a treatment agent directly via the skin to avoid the challenges presented by oral delivery and use of needles. The treatment of a disease can be programmed to utilize receptor turnover rates to optimize delivery of the treatment agent. The delivery of a therapeutic agent using pulsed delivery may also be programmed to “ramp down” the amount of drug given over time or gradually extend the drug delivery time interval.
Category 2: The pulsatile agent delivery system may also be configured to include a feedback system, which measures and monitors the therapeutic agent or a biologic marker to determine if additional agent administration is required.
Category 3: The agent delivery system may also be utilized in cosmetic applications to provide needleless cosmetic treatments. This application would involve an altered device shape to fit the facial feature treated or the whole face. This application utilizes an external electric field to facilitate perfusion of the agents across the skin.

The applications for the agent delivery system are not meant to be limitations, those skilled in the art understand that the type of agent and therapeutic dosing requirements would guide the configuration of the agent delivery system for a specific therapy.

### BRIEF DESCRIPTION OF THE DRAWINGS

Other advantages of the present invention can be readily appreciated as the same becomes better understood by reference to the following detailed description when considered in connection with the accompanying drawings wherein:

FIG. 1A illustrates an embodiment of the present invention of a one-time use device, wherein the device includes a collection chamber and several assaying chambers, and 1B illustrates another embodiment of the present invention of a system, wherein the system includes at least one sensor connected to a remote display system and at least one collection chamber, at least one separation chamber, and at least one sensing chamber in communication with the other chambers through micro-conduits;

FIG. 2 shows the CAD layout of the chambers wherein two chips constitute the top and bottom of the device;

FIG. 3 shows the complete mask layout;

FIGS. 4A and 4B shows the cross-section of the assembled chip;

FIG. 5 shows top and bottom pieces of the chamber, mated together;

FIG. 6 shows a thick bead of photoresist material at the corner of the etched;

FIG. 7 shows that the vaporized OP was bubbled through an appropriate buffer solution, causing the OP to dissolve back into the liquid to be assayed;

FIG. 8 is a graph that shows the activity of the enzyme was determined by measuring the change in absorbance (or slope) after one month and two months of storage at −4 C;

FIG. 9 shows that the separation of the enzyme globule from the plastic substrate caused the effective surface area of the immobilized enzyme to increase, enabling more substrate to react with the enzyme;

FIG. 10 shows that there was significant suppression of enzyme activity in the 2P1 immobilized enzyme wells;

FIG. 11 shows the results of a kinetic protocol was created on the photometric micro-titer plate reader to take an absorbance reading at 405 nm every minute for 10 minutes, and compute an average slope;

FIG. 12, show almost identical slopes for control and plasma cholinesterase, confirming the capacity of the BTC substrate to detect cholinesterase activity in plasma;

FIG. 13 shows that acetylcholinesterase from RBC lysate had significant activity (slope=53.6 mOD/min) when the AcTC substrate was used, whereas there was significantly less activity (slope=13.7 mOD/min) for the reaction using the BTC substrate;

FIG. 14 shows the effect of selective inhibition on plasma samples that were treated with quinidine (20 μM), the inhibitory effect was observed only when BTC was used;

FIG. 15 shows the effect of selective inhibition on plasma samples that were treated with quinidine (20 μM), the inhibitory effects of cholinesterase activity with and without quinidine was observed;

FIG. 16 shows that diluted and undiluted plasma showed cholinesterase activity using substrate reagents that were dried and spotted individually;

FIG. 17 shows that the present invention can include a detection chamber that can fit into a conventional 96 well plate and read using a conventional spectrophotometer;

FIG. 18 shows that absorbance increased in a linear manner for the wells containing plasma and also shows that a detectable color change occurred;

FIG. 19 shows the reliability of the sampling and immunoassay analysis and a correlation to literature values, the pre melatonin saliva values were averaged (n=5, MEAN=17.5+/−8.4 pg/mL);

FIG. 20 shows that in normal adults, serum melatonin concentrations are highest during the night (about 60 to 200 pg/mL) and lowest during the day (about 10 to 20 pg/mL) and that these concentrations are well within the melatonin standard curve as determined by amperometry;

FIG. 21 shows a glucose (Sigma, Cat. No. EC No 200-075-1, Lot No. 41 K0184) standard curve that was prepared with concentrations ranging from 50 mg/dL to 400 mg/dL;

FIG. 22 shows that the diode acts as a quarter wave stack, enhancing the signal at certain wavelengths;

FIG. 23 shows that the response of the diodes is linear to the amount of incident power;

FIG. 24 shows optical chemical sensors reproduced on silicon chips by incorporating a photo-diode with an optical membrane on top of the diode;

FIG. 25 is a photomicrograph of the 2 μm sensor array;

FIG. 26 shows a different size sensor array chips bonded in a ceramic carrier;

FIG. 27 shows a schematic of the sensor array;

FIG. 28 shows alternative sensor array configurations;

FIG. 29 shows an inhibition of the ChE activity that was demonstrated in the presence of OP;

FIGS. 30A, 30B, 30C, and 30D show a variety of different support mechanisms located within a chamber of the present invention;

FIGS. 31A, 31B, and 31C show a variety of support mechanism spacing within a chamber of the present invention;

FIGS. 32A and 32B are CAD drawings of a transdermal sampling chamber of the present invention;

FIG. 33 shows a microfluidic system of the present invention;

FIG. 34 shows a microfluidic actuator and microfluidic valve of the microfluidic system of the present invention;

FIG. 35 is a cross-sectional layout of the fluid analyzing device;

FIG. 36 is a cross-sectional layout of the fluid analyzing device with a separation membrane (electrolyte polymer membrane);

FIG. 37 is a cross-sectional view of a system of the present invention including a removable membrane interface chamber;

FIG. 38 is a schematic view of a CAD layout of the fluid analyzing device and the fluid analyzing system, this chip measures 8 mm×4 mm×2 mm, the membrane interface chamber resides underneath the chip;

FIG. 39 is a cross-sectional view of the fluid delivery device with supports;

FIG. 40 is a cross-sectional view of the fluid delivery device, with an electrolyte polymer membrane;

FIG. 41 is a schematic view of the fluid analyzing system on one body portion;

FIG. 42 is a cross-sectional view of the fluid analyzing system on one body portion;

FIG. 43 is a cross-sectional view of the fluid analyzing system on two body portions;

FIG. 44 is a dose-response curve of closed loop delivery vs. standard methods of delivery;

FIG. 45 is a back view of a mock-up of a patch with pulsatile delivery, approximately 2 cm in diameter (the size of a band-aid);

FIG. 46 is a flow chart of a model-based controller;

FIG. 47 illustrates a comparison of lithium delivery methods in hairless mice; and

FIG. 48 illustrates a software interface.

### DETAILED DESCRIPTION OF THE INVENTION

In an embodiment of the present invention, an agent delivery device is provided. The agent delivery device of this embodiment is useful for administering a biologically compatible agent to a patient. The agent delivery device includes an agent delivery reservoir containing the agent to be administered to the patient. An electrolyte receives at least a portion of the agent from the agent delivery reservoir. The electrolyte is mixed with the agent to form an electrolyte-agent mixture that is contained in the reservoir. Moreover, the electrolyte-agent mixture traps the agent until electric current is applied thereto. The device also includes an agent delivery surface in communication with the electrolyte. In a refinement, the agent delivery device includes one or more additional delivery surfaces. The agent delivery surface contacts the patient and delivers agent received from the reservoir to the patent. A controller in communication with the electrolyte-agent mixture provides a series of control pulses to the electrolyte. Each pulse allows the device to administer a portion of the agent to the patient. The series of pulses provides a temporally varying concentration of agent in the patient. In a variation, the electrolyte comprises an iontophoretic electrically conductive material. In a further refinement, the electrolyte is polymeric. The term iontophoretic electrically conductive material means any material that exhibits iontophoretic behaviour.

In a variation of the present embodiment, the temporally varying concentration of agent includes a plurality alternating agent concentration maxima and minima with the maxima and minima differing by a predetermined amount. In a further refinement, the maxima and minima differ by at least 5%, 10%, 20%, 30%, 40% and 50% in order of increasing preference. In some refinements, the temporally varying concentration of agent is matched to the turnover of cell receptors for the agent. In another refinement, the temporally varying concentration of agent is matched to the life-cycle of an invading bacteria or parasite In another refinement, the temporally varying concentration of agent is such that agent concentration maxima in the patient is increased over time. In a further refinement, the series of pulses provide a temporally increasing agent concentration maxima in the patient for a first predetermined time period. In still a further refinement, the series of pulses provides a temporally decreasing agent concentration maxima in the patient for a second predetermined time period that occurs after the second time period. In another refinement, the temporally varying concentration of agent is such that agent concentration maxima in the patient is decreased over time

As set forth above, the agent delivery device of the invention includes a digital controller and a memory accessible to the digital controller. An algorithm for controlling the electrolyte is encoded in the memory such that the algorithm may be executed by the digital controller. In a refinement, one or more intervals of the series of controlled pulses are varied over time via the controlling algorithm. In another refinement, the amplitudes of the series of controlled pulses are varied via the controlling algorithm. In still another variation, the duration or width of the series of controlled pulses is varied via the controlling algorithm.

In another variation, the agent delivery devise further includes a sensor system for determining the concentration of the agent in the patient. In a refinement of this variation, such a sensor system is advantageously used in a feedback loop to the controller. In such a feed back loop, information from the sensor system is used to adjust the concentration of the agent or one more additional agents in the patient.

In another embodiment of the present invention, a method of delivering a biologically compatiable agent to a patent is provided. The method of this embodiment utilizes the agent delivery device set forth above. The method of this embodiment comprises contacting the patient with the agent delivery surface and then operating the controller to administer the agent to the patent.

A number of different compositions may be used for the agent in the present inventions. Examples of such compositions include anti-malarial agents, hormones, antiretroviral drug, antibiotic drugs, antipsychotic drugs (e.g., lithium), addictive agents, chemotherapeutic cancer agent, cosmetic anti-wrinkle agent, naturally occurring or synthetic hydrophilic or hydrophobic agents, the agent comprises an analgesic, and the like. Specific examples of anti-malarial agents include amodiaquine, artemether, artemisinin, artesunate, atovaquone, cinchonine, cinchonidine, chloroquine, doxycycline, halofantrine, mefloquine, primaquine, pyrimethamine, quinine, quinidine, sulfadoxine, and combinations thereof. Specific examples of hormones include gonadotropin releasing hormones (GnRH), estradiol, progesterone, growth hormone, thyroid stimulating hormone (TSH) prolactin, human parathyroid hormone buserelin, insulin, and combinations thereof. Specific examples of antiretroviral drugs include abacavir, didanosine, indinavir, lamivudine, nevirapine, ritonavir, saquinavir mesylate, zalcitabine, zidovudine, and combinations thereof. Specific examples of antibiotic drugs include ampicillin, azithromycin, doxycycline, erythromycin, penicillin, tetracycline, and combinations thereof. Specific examples of addictive agents include nicotine, morphine, methadone, and combinations thereof. Specific examples of chemotherapeutic cancer agents include Buserelin, Taxol, and combinations thereof. Specific examples of cosmetic anti-wrinkle agents include acollagen, collagen-glycosaminoglycan, polytetrafluoroethylene, poly-L-lactide and poly(ethyleneoxide)-poly(butyleneterephthalate), polyglactin, polyglycolic acid, biosynthetic materials, hydrocolloid-like materials, and combinations thereof. Specific examples of analgesic agents include non-steroidal anti-inflammatory drugs, steroids, COX-1 inhibitors, COX-2 inhibitors, and combinations thereof.

Generally, the present invention provides a completely automated, miniaturized agent delivery system/device 10 capable of detecting, monitoring, and delivering different types of agents from or into a minute amount of fluid. The present invention can determine a subject's reaction to various agents, analyze trends, perform comparisons among a normalized standard of people, determine tolerance levels of a subject, and/or treat the disease or condition accordingly. More specifically, the present invention is a micro-electro-mechanical system (MEMS) based agent delivery device 10 with optionally integrated fluid acquisition or microfluidic system 11 and external monitoring system 44.

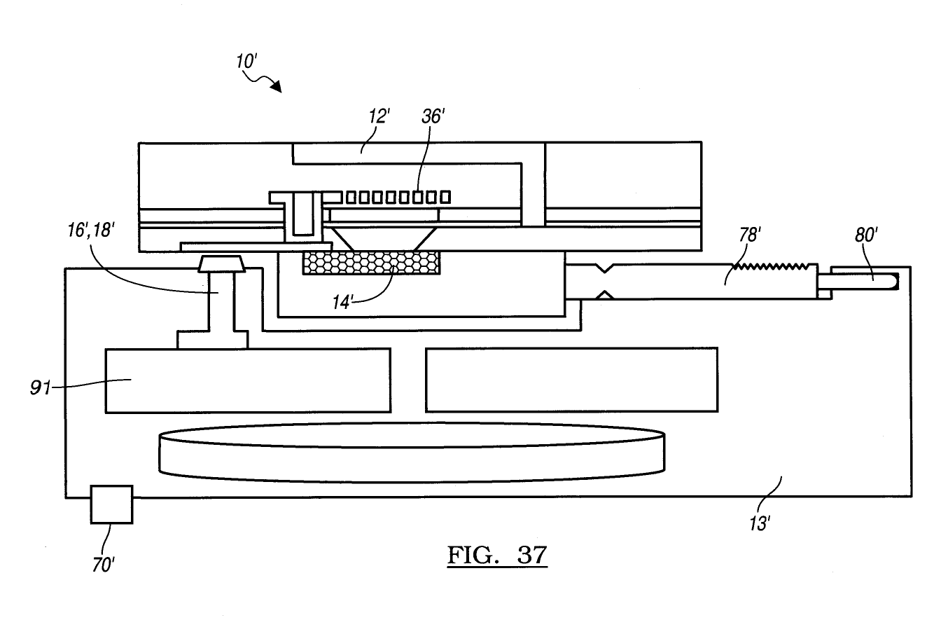

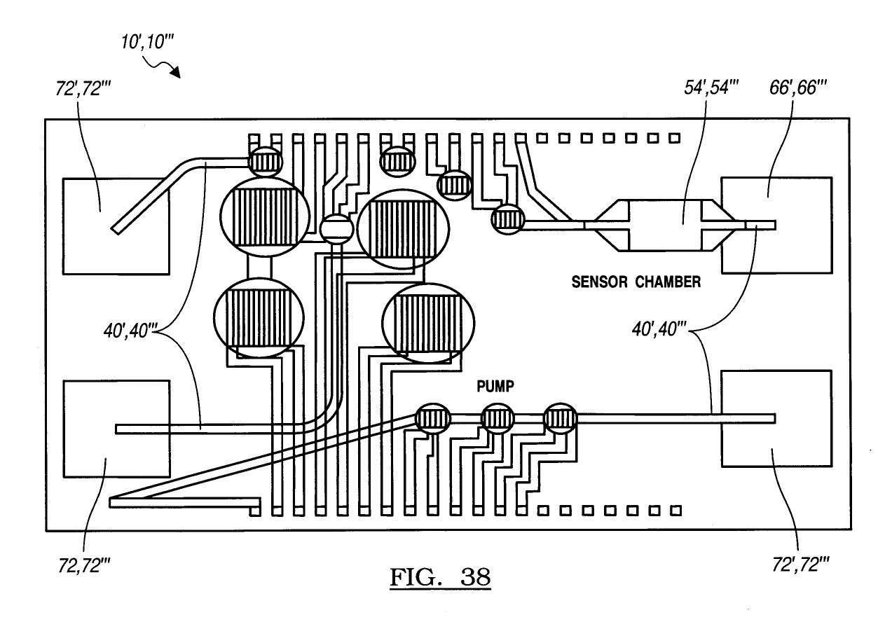

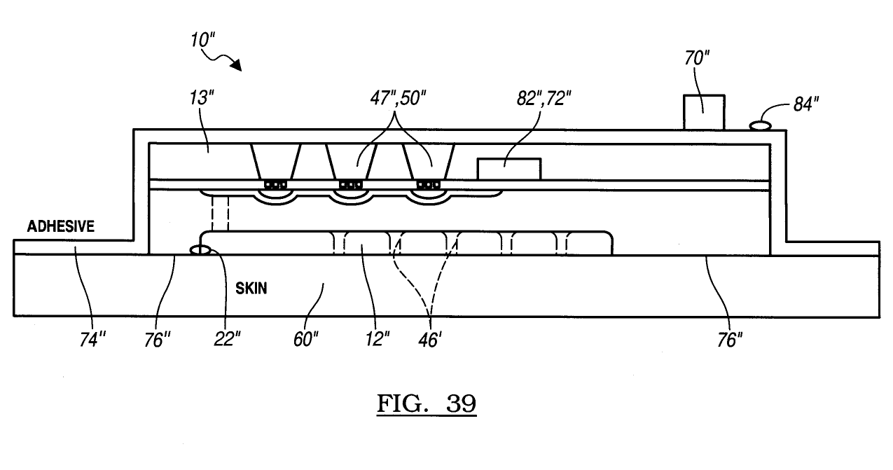

This agent delivery device 10 is small and non-invasively monitors interstitial fluids that are in equilibrium with the concentration in blood. The agent delivery device 10 contains a low power micro-fluidic pump for transporting fluid sample to the sensors, micro-fluidic conduits and valves for routing sample and calibration solutions, silver/silver chloride (Ag/AgCl) reference electrodes for electrical stimulation of the skin, microscopic semiconductor sensors to detect ions and chemicals, and electronic circuitry to control the pumps and valves as well as to provide integration with existing data-logging and telemetry systems. FIG. 37 depicts a cross-section of the final device with sampling and sensor chambers, waste reservoir, and three polysilicon heaters with membrane actuators to act as the peristaltic pump.

The agent delivery device 10 of the present invention can incorporate microscopic, interdigitated sensor arrays (potentiometric, amperometric, and optical) able to transduce compositions in less than 1 μl sample volumes. Membranes are placed onto the sensing arrays to confer specificity to the desired agent (in combination with other molecules). The agent delivery device 10 is preferably formed utilizing a micro screen printer. Because of their extremely small size, arrays of these sensors provide the ability to utilize more than one electrode for statistical control, as well as providing the ability to transduce dozens of molecules simultaneously.

Unlike the prior art systems, the agent delivery device 10 of the present invention allows delivery of hydrophilic as well as hydrophobic molecules, such as antibiotics. The agent delivery device 10 is smaller (less than 2 cm²), less expensive to manufacture, and utilizes an electrolyte polymer to trap the drug in large quantities and release it, as square-wave pulses, only when iontophoretic current is applied. The agent delivery device 10 is fully programmable utilizing on-chip custom CMOS circuitry, thus allowing it to be programmed for any pulse length and frequency regime. Using a programmed algorithm, the timing and duration of each pulse can be changed throughout the treatment to provide the agent delivery pattern sufficient to provide appropriate protection without overdosing, underdosing, creating resistance to the drug, or any of the other known side effects.

The transdermal delivery of drugs, by diffusion through a body surface, offers improvements over more traditional delivery methods, such as subcutaneous injections and oral delivery. Transdermal drug delivery also avoids the hepatic first pass effect encountered with oral drug delivery. Generally the term “transdermal” when used in reference to drug delivery, broadly encompasses the delivery of an agent through a body surface, such as the skin, mucosa, nails or other body surfaces (e.g., an organ surface) of an animal.

When iontophoresis has been used to obtain transdermal interstitial fluid samples in the prior art devices, a troublesome tingling sensation was experienced by patients from the large area electrodes employed in the study (10 cm²). Such problems are overcome by the agent delivery device 10 of the present invention, which has a smaller area electrode (1 cm²) with an equivalent current density that does not produce as significant a “side-effect”; however, the reduced surface area results in a significantly reduced volume of drawn interstitial fluid. By reducing the test volume required for analysis by three orders of magnitude, the surface area of the agent delivery device 10 can be significantly reduced without affecting the ability of the agent delivery device 10 to perform the necessary functions. The agent delivery device 10 is able to be so much smaller because of the microscopic semiconductor sensor arrays. The agent delivery device 10 continuously monitors interstitial fluid in near real-time, is a small patch, approximately 10 mm×10 mm, that contains low power micro-fluidic pump for transporting fluid samples, micro-fluidic conduits and valves for routing interstitial fluid samples and calibration solutions, platinum electrodes for electrical stimulation of the skin, microscopic semiconductor sensor arrays to detect glucose, ions, and other analytes, and electronic circuitry to control the pumps and valves as well as to provide integration with existing data-logging, telemetry, and device (pump) control systems. A schematic view of the complete micro-fluidic system, including transdermal sampling chamber and sensor array chamber, and a CAD drawing of the device is shown in the figures. Platinum electrodes can be integrated into the sampling chamber to facilitate iontophoretic methods to sample interstitial fluids.

The method of delivering drugs and metabolites to patients using the agent delivery device 10 of the present invention follows normal physiological concentrations patterns, as opposed to super- or pharmaco-physiological concentrations and patterns, the timing of which is based on systemic factors including receptor dynamics, drug clearance, drug half-life, etc. The delivery timing is based on closed-loop feedback via monitoring of the actual delivered molecule (i.e., lithium or nicotine) or by monitoring of a second indicator molecule (i.e., glucose monitoring for insulin administration). This provides “on-demand” delivery of the agent. Further, the “on-demand” delivery of agents/drugs maintains the body load to the therapeutic level as opposed ton the great oscillations present when administered orally or via injection. The invention provides pulsatile delivery of the agent/drug and continuous “ramp-down” capability, controlled automatically. With either form of feedback monitoring, the administration of the agent occurs objectively, without requiring a subjective analysis. This aids in limiting overdosing or creating an addiction to an agent, because the administration is based upon readily ascertainable bodily events that can be tested/analyzed objectively. Since only the necessary amount of agent is being administered, lower amounts of agents can be administered. The end result of the delivery methods are fewer side effects, less drug resistance, less increased tolerance to agents, and increasing the number of individuals that are able to benefit from the agents.

### DEFINITIONS

Like structure among the several defined embodiments are indicated by primed numbers.

The terms “chamber 12,” “sampling chamber 12,” “reacting chamber 12,” and “sensor chamber 12” are defined as an enclosed cavity wherein fluids are retained.

The term “agent” is defined as a traceable biological or chemical component. As used herein, an “agent” is meant to include, but is not limited to environmental agents, blood markers, antigens, pesticides, drugs, chemicals, toxins, PCBS, PBBS, lead, neurotoxins, blood electrolytes, metabolites, analytes, NA+, K+, CA+, urea nitrogen, creatinine, biochemical blood markers and components, ChE, AChE, BuChe, tumor markers, PSA, PAP, CA 125, CEA, AFP, HCG, CA 19-9, CA 15-3, CA 27-29, NSE, hydroxybutyrate, acetoacetate, anti-malarial drugs such as amodiaquine, artemether, artemisinin, artesunate, atovaquone, cinchonine, cinchonidine, chloroquine, doxycycline, halofantrine, mefloquine, primaquine, pyrimethamine, quinine, quinidine, and sulfadoxine; anti-biotic drugs such as ampicillin, azithromycin, doxycycline, erythromycin, penicillin, and tetracycline; anti-retroviral drugs such as abacavir, didanosine, indinavir, lamivudine, nevirapine, ritonavir, saquinavir mesylate, zalcitabine, and zidovudine; nicotine; gonadotropin releasing hormone (GnRH), estradiol, progesterone, growth hormone, morphine, methadone, lithium, and insulin, and any other similar agents known to those of skill in the art.

The term “monitoring” is defined as testing, sampling, detecting, sensing, and/or analyzing an agent. Testing can either determine the presence of the agent or identify the agent itself. Moreover, testing includes both quantification and qualification of the agent.

The term “antigen” or “immunogen” is defined as any substance that is capable of inducing the formation of antibodies and reacting specifically in some detectable manner with the antibodies so induced. Not all antigens however, are immunogens. Examples of an “antigen” include, but are not limited to, immunogens such as viruses, bacteria, microbes, pathogens, HIV, hepatitis, anthrax, cholera, Q-fever, smallpox, tuberculosis, and any other similar biological agents or pathogens known to those of skill in the art.

The term “subject” or “patient” as used herein is defined as, but is not limited to, humans and animals.

The term “fluid” or “fluids” as used herein is meant to include, but is not limited to, blood, plasma, saliva, urine, sputum, feces, interstitial fluids, tears, sweat, water, and any other similar bodily fluids or other fluids known to those of skill in the art.

The term “label” as used herein is defined as a device that enables the quantitation and quantification of an agent. Examples of labels that can be used in connection with the present invention include, but are not limited to, chemiluminescent labels, luminescent labels, fluorescent labels, calorimetric labels, including, but not limited to, absorption, bioluminescence, and fluorescence, radiolabels, and enzyme labels.

The term “working electrode 16” as used herein is defined as, but is not limited to, an electrode that supplies the potential source for affecting oxidation and/or reduction.

The term “counter electrode 18” is defined as an electrode paired with a working electrode 16, through which an electrochemical current passes equal in magnitude and opposite in sign to the current passed through the working electrode. In the context of the invention, the term “counter electrode 18” is meant to include counter electrodes 18 that can have the dual function as a potentiometric reference electrode (i.e. a counter/potentiometric electrode). The counter electrode 18 is an electrode at which an analyte is electrooxidized or electroreduced with or without the agency of a redox mediator.

The term “amperometric electrochemical sensor” is defined as a device configured to detect the presence and/or measure the concentration of an analyte via electrochemical oxidation and reduction reactions on the sensor. These reactions are transduced to an electrical signal that can be correlated to an amount or concentration of analyte.

The term “electrolysis” is defined as the electrooxidation or electroreduction of an agent either directly at an electrode or via one or more electron transfer agents. An example of this includes, but is not limited to, using glucose oxidase to catalyze glucose oxidation creating oxidized glucose and peroxide, where the peroxide is being measured.

The term “facing electrodes” is defined as a configuration of the working and counter electrodes 16 and 18 in which the working surface of the working electrode 16 is disposed in approximate apposition to a surface of the counter electrode 18.

The term “measurement zone 28” is defined as a region of the sample chamber sized to contain only that portion of the sample that is to be interrogated during an analyte assay.

The term “non-leachable compound” or “non-releasable compound” is a compound, which does not substantially diffuse away from the working surface of the working and/or counter electrodes for the duration of an analyte assay.

The term “redox mediator” is defined as an electron transfer agent for carrying electrons between the analyte and the working electrode, either directly or via a second electron transfer agent.

The term “reference electrode 24” is defined as an electrode used to monitor and account for voltage drop due to medium resistance in amperometric sensors, and supplies a reference potential for comparison in potentiometric electrodes.

The term “second electron transfer agent” is defined as a molecule that carries electrons between the redox mediator and the analyte (See example above).

The term “sorbent material” is defined as a material that wicks, retains, or is wetted by a fluid sample in its void volume and does not substantially prevent diffusion of the analyte to the electrode.

The term “working surface 26” is defined as that portion of the working electrode, which is coated with redox mediator and configured for exposure to sample.

The term “actuator 30” as used herein is defined as, but is not limited to, a device that causes something to occur. The actuator 30 activates the operation of a valve, pump, villi, fan, blade, or other microscopic device. Typically, the actuator of the present invention affects fluid flow rates within a chamber.

The term “closed cavity 52” as used herein is defined as, but is not limited to, a sealed cavity that contains a liquid or solid expanding mechanism 32 that is expanded or vaporized to generate expansion or actuation of a flexible mechanism 34. The closed cavity must be completely sealed in order to contain the expansion therein, and must be flexible on at least one side.

The term “expanding mechanism 32” as used herein is defined as, but is not limited to, a fluid capable of being vaporized and condensed within the closed cavity enclosed by the flexible mechanism 34. The expanding mechanism 32 operates upon being actuated or heated. The expanding mechanism 32 includes, but is not limited to, water, wax, hydrogel (solid or non-solid), hydrocarbon, and any other similar substance known to those of skill in the art. Condensation of the expanding mechanism 32 occurs when the heat, which is generated to induce expansion of the expanding mechanism, is removed by a surrounding medium such as a gas, liquid or solid. Then, once condensation occurs, contraction of the flexible mechanism 34 takes place.

The term “flexible mechanism 34” as used herein is defined as, but is not limited to, anything that is capable of expanding and contracting with the vaporization and condensation of the expanding mechanism. The flexible mechanism 34 must be able to stretch without breaking when the expanding mechanism 32 is vaporized. The flexible mechanism 34 is made of any material including, but not limited to, silicone rubber, rubber, polyurethane, PVC, polymers, combinations thereof, and any other similar flexible mechanism 34 known to those skilled in the art.

The term “heating mechanism 36” as used herein is defined as, but is not limited to, a heating device that is incorporated with the actuator 30 of the present invention. The heating mechanism 36 generates heat to induce expansion of the expanding mechanism. The heating mechanism 36 is disposed adjacent to the flexible mechanism 34 in order to turn on and off and maintaining on and off selective expansion of the expanding mechanism 32. The heating mechanism 36 can be powered using any power source known to those of skill in the art. In the preferred embodiment, the heating mechanism 36 is powered by a battery. However, both AC and DC mechanisms are used to minimize power requirements. Generally, the heating mechanism 36 is formed of materials including, but not limited to, polysilicon, elemental metal, silicide, or any other similar heating elements known to those of skill of the art. Moreover, the heating mechanism 36 is disposed within a medium such as Si0₂ or other solid medium known to those of skill in the art.

The term “temperature sensor 38” as used herein is defined as, but is not limited to, a device designed to determine temperature. A resistive temperature sensor 38 is made from material including, but is not limited to, polysilicon, elemental metal, silicide, and any other similar material known to those of skill in the art. Thermocouple temperature sensor 38 can also be used. Typically, the temperature sensor 38 is situated within or near the heating element of the heating mechanism 36.

The terms “micro-conduit,” “microfluidic conduit,” and “conduit 40” as used herein are defined as, but not limited to, any type of tube, pipe, planar channel, conduit, or any other similar conduit known to those of skill in the art. The conduit has a wall mechanism made from material including, but not limited to, silicon, glass, rubber, silicone, plastics, polymers, metal, and any other similar material known to those of skill in the art. In one embodiment of the microfluidic valve, the conduit encompassing the micro-actuator is etched out of glass in a nearly hemispherical shape. A variety of conformations of spherically cut patterns (i.e. ⅓ of a sphere, ½ of a sphere, etc.) with differing radii and footprints are employed to provide different valving characteristics.

The device of the present invention can be composed of numerous materials including, but not limited to, plastic, silicone, glass, metals, alloys, rubber, combinations thereof, or any other similar material known to those of skill in the art.

Typically, the device of the present invention is manufactured by chemical etching methods known to those of skill in the art. Thus, the chambers and micro-conduits of the present invention can be etched into a base material of silicon or glass. The chambers are made out of material that is sandwiched between pieces of silicon, glass or membranes. Further, the present invention can be made by utilizing glues and other securing methods and materials known to those of skill in the art. Fabrication of the microfluidic system components is based upon the development of a process flow. The fabrication process utilizes bulk silicon micro-machining techniques to produce the isolation windows, and thick film screen-printing techniques, spin coating, mass dispensing, or mechanical dispensing of actuation membranes.

Alternatively, the chambers and conduits can be produced from plastic by injection molding, micro-milling, or soft lithography. The materials of the present invention can be modified or altered according to the specific design required. Moreover, the device of the present invention can vary in size, shape, and configuration without departing from the spirit of the present invention.

The agent delivery device 10 of the present invention has numerous advantages over currently existing devices. For instance, the present invention is minimally invasive and measures nanoliter and microliter amounts of fluids and not milliliter amounts.

The agent delivery device 10 of the present invention can perform various assays such as ELISA, but also is capable of performing chromatographic separations. The agent delivery device 10 of the present invention is capable of performing various tests on a single, small unit sensor system without the aid, or need, of external equipment (i.e., laboratory-on-a-chip). However, the device can be optionally linked to an external electrical source, power source, computer unit, or palm pilot as desired by the user either directly with wires or via telemetry. The agent delivery device 10 of the present invention can also be constructed as an instrumentless device and can provide easily readable visual indicia of a positive and/or negative test.

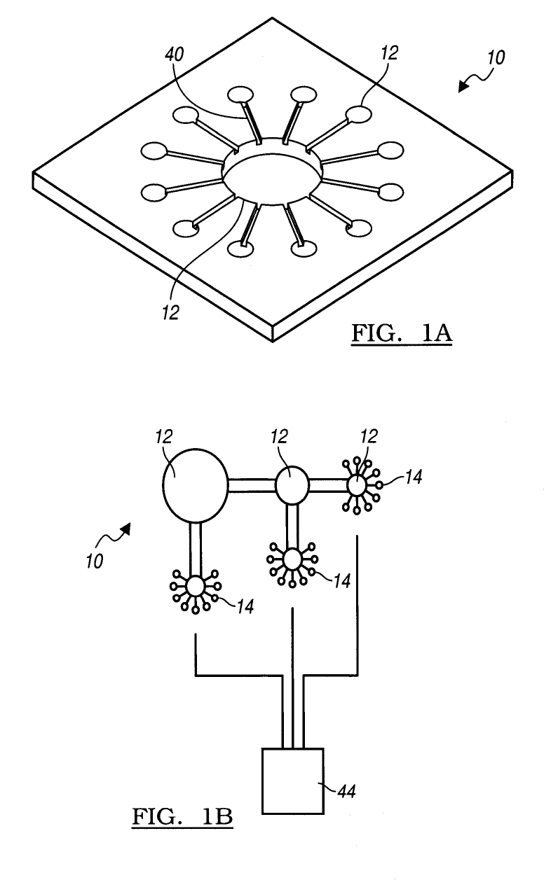

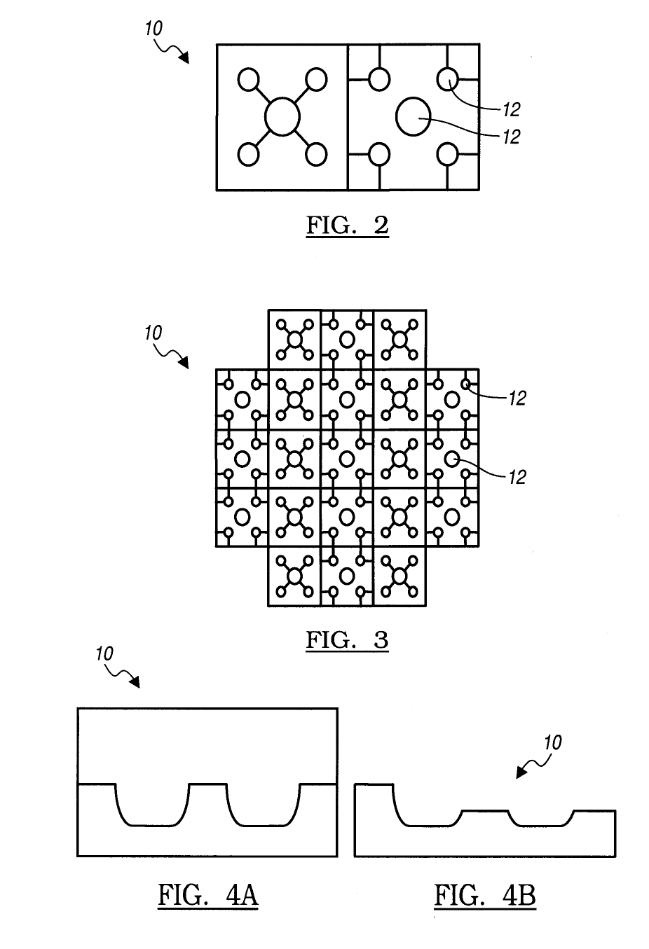

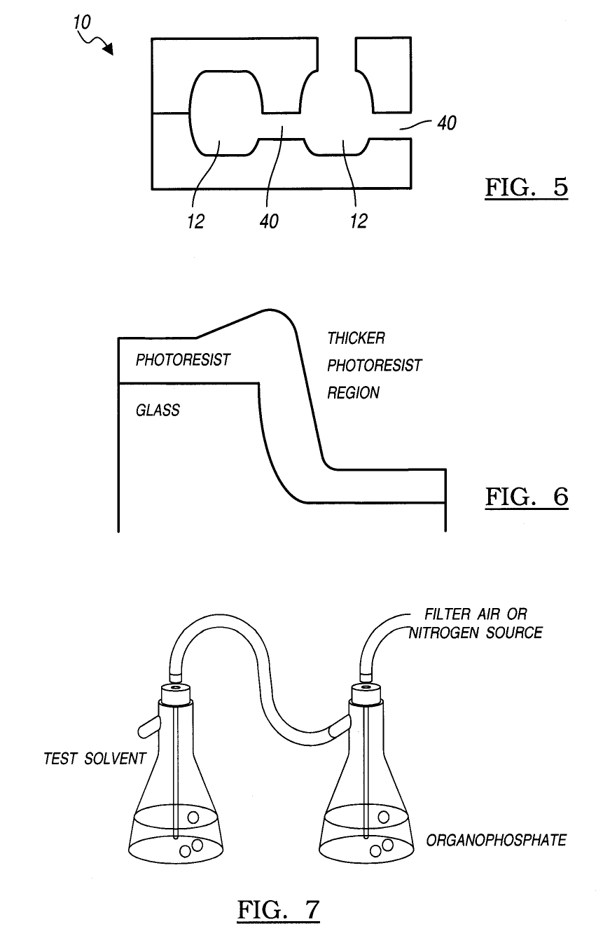

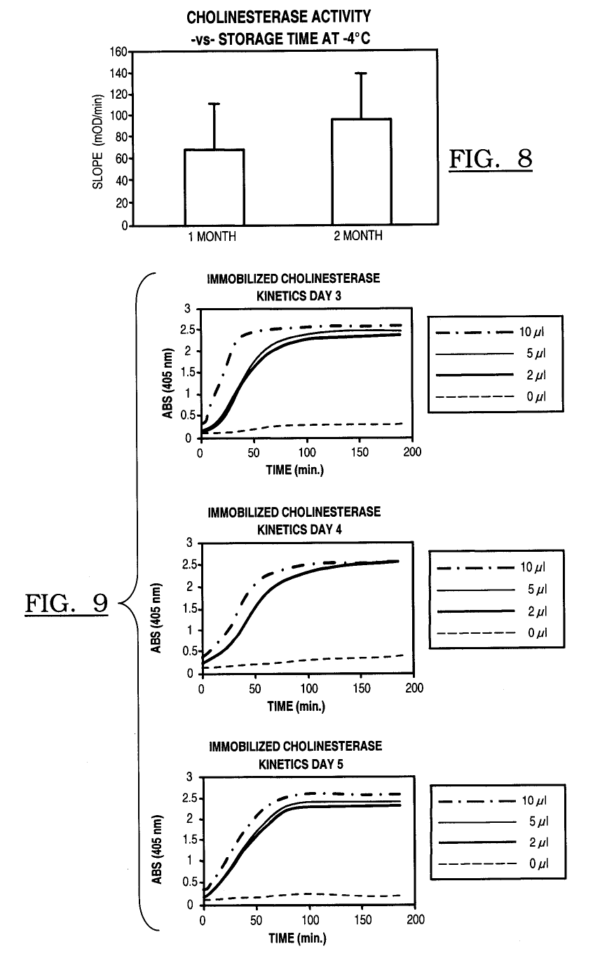

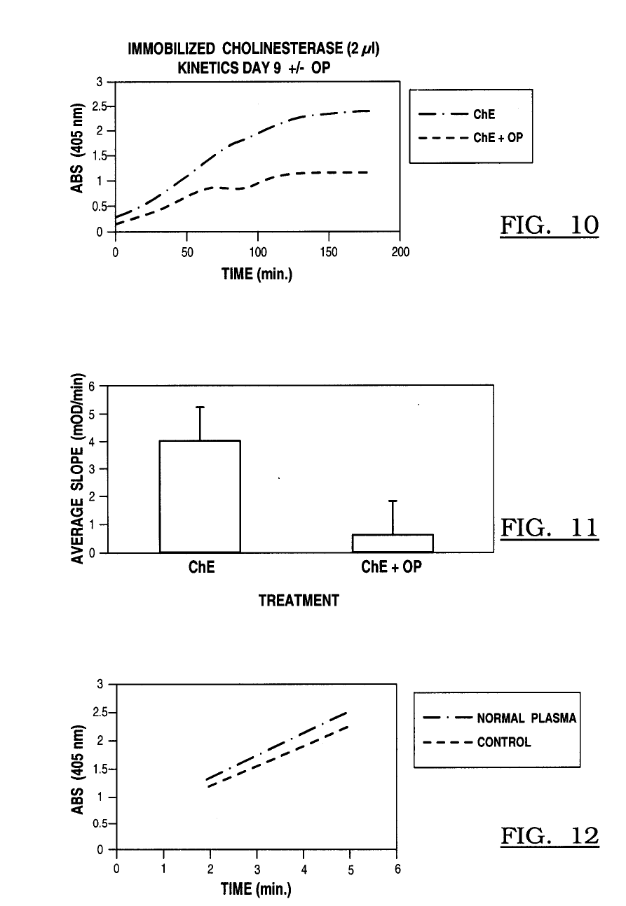

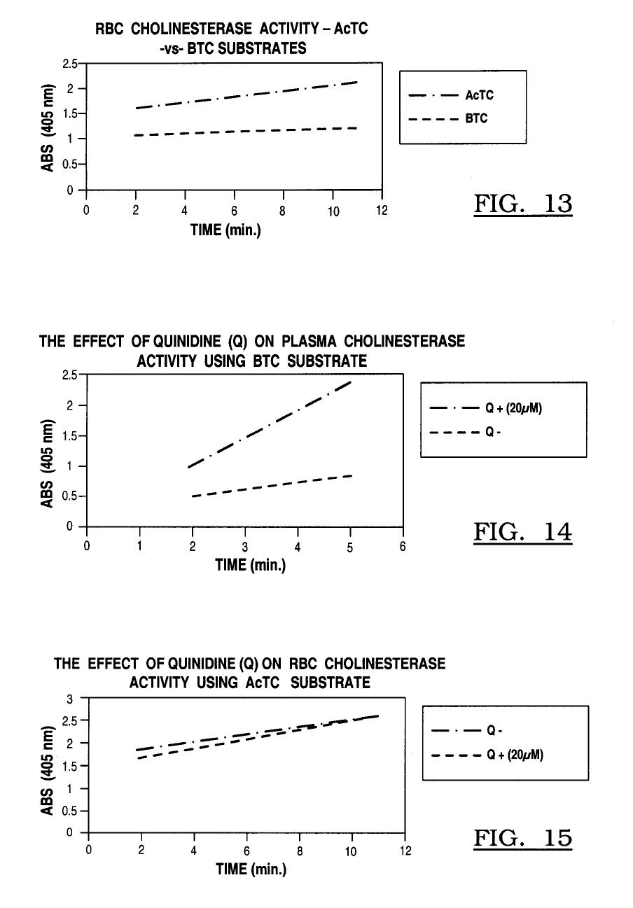

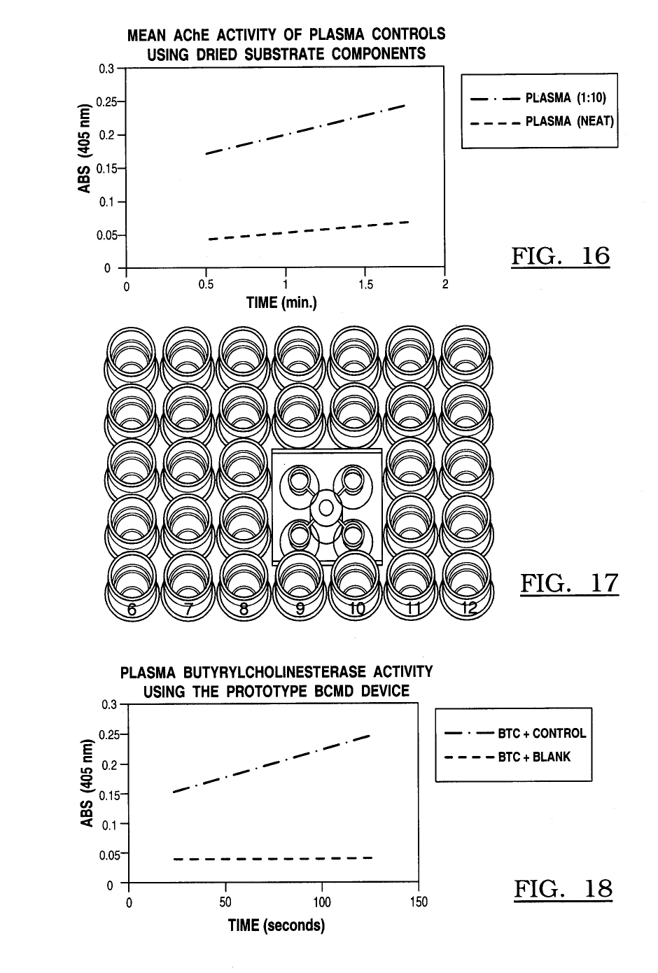

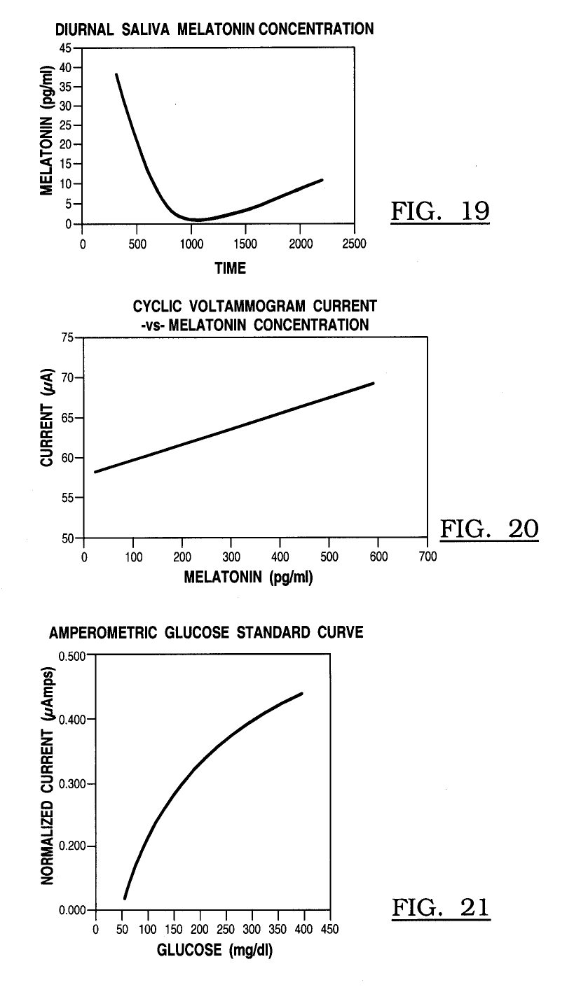

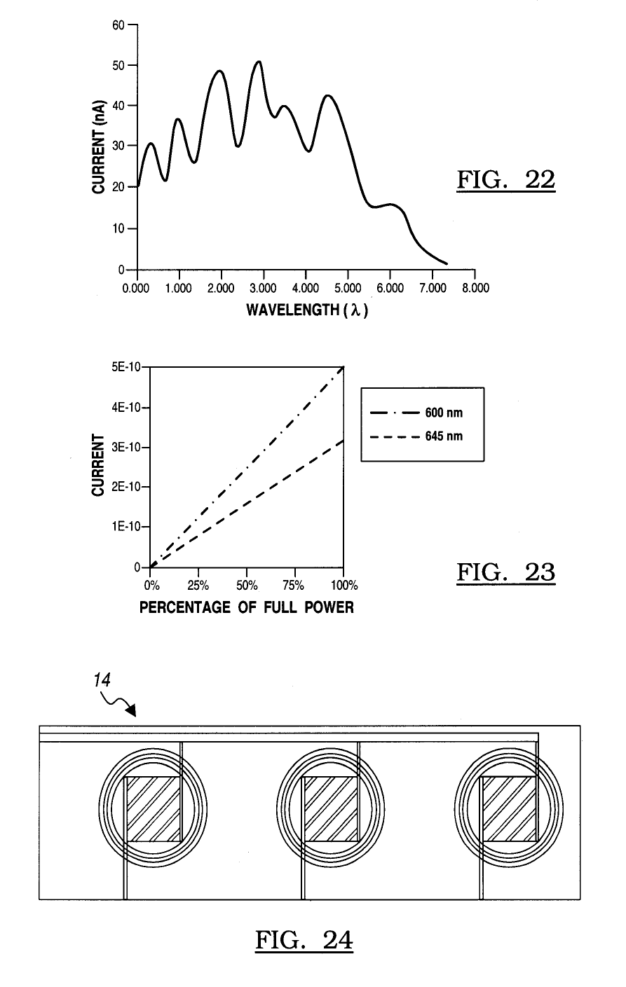

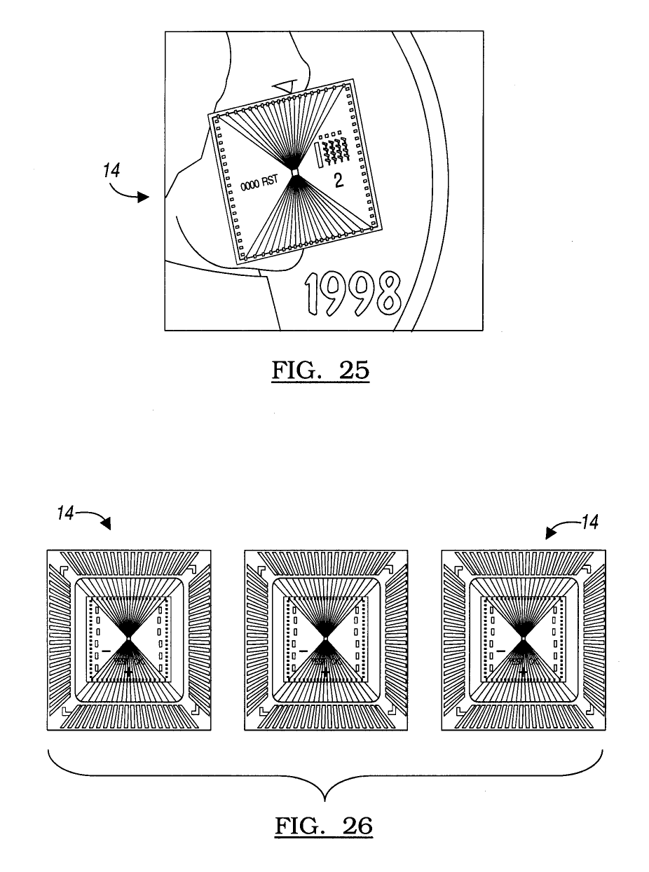

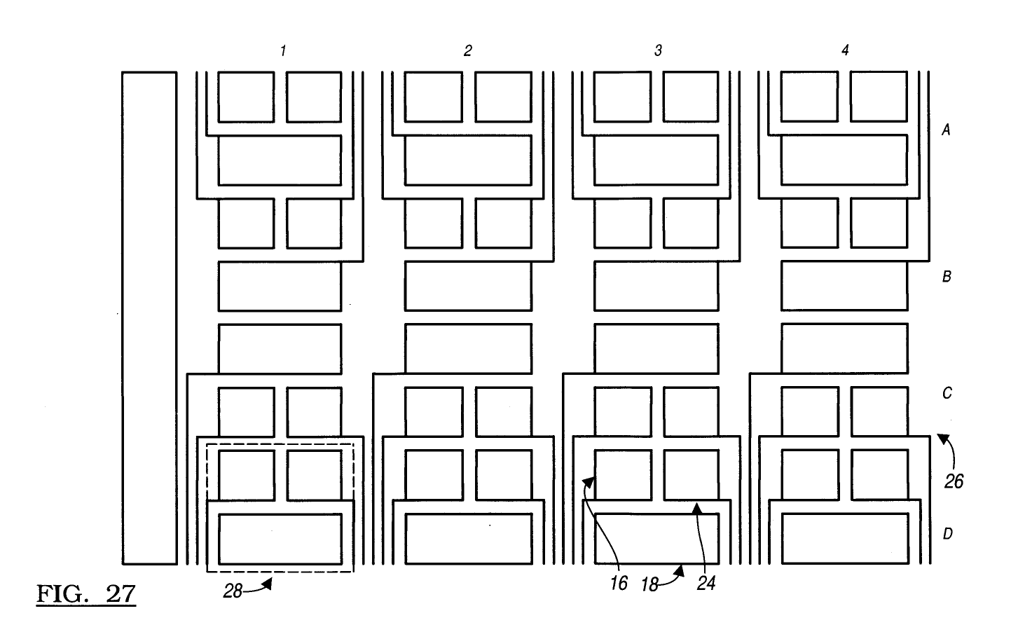

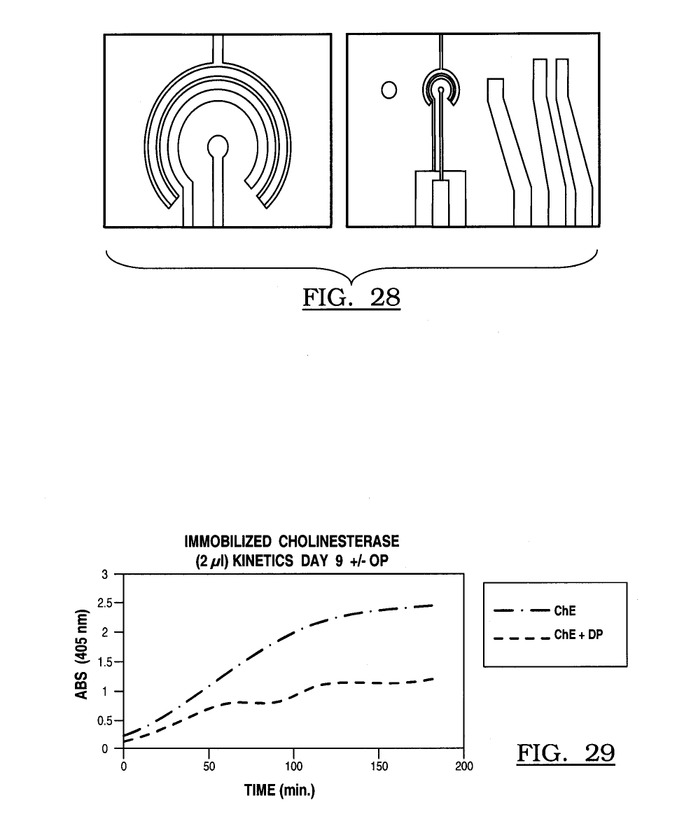

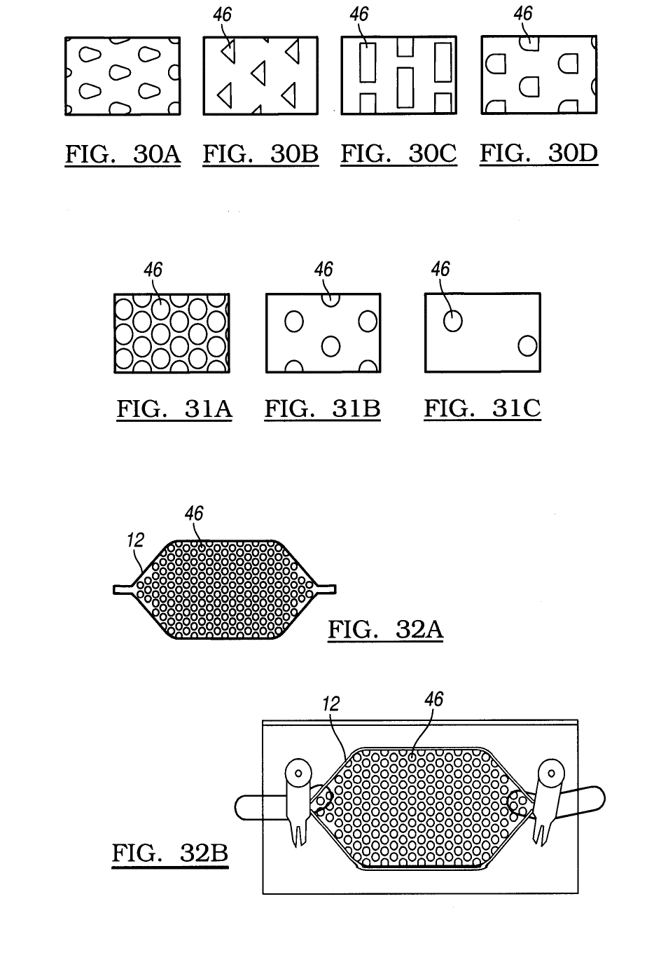

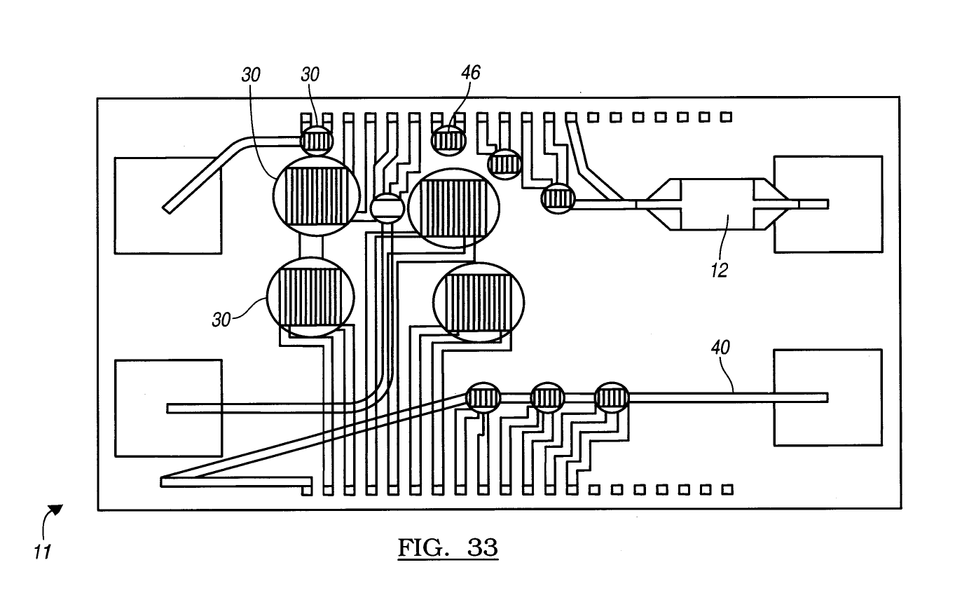

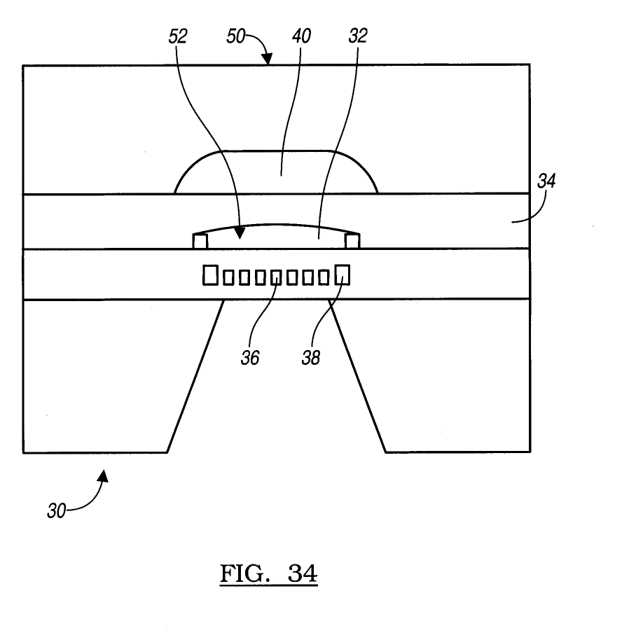

The present invention has additional advantages in that it is capable of having either a single or numerous chambers 12 (FIGS. 1 and 2). Various reactions of the fluid can take place in one chamber 12 or various other chambers 12. Movement of the fluids occurs through micro-conduits 40 connecting the chambers 12. Alternatively, reactions can take place between chambers 12 and within the micro-conduits 40 themselves. For example, a fluid can be added to a sampling chamber 12, treatment of the fluid then occurs along the micro-conduit, and the results are obtained at an end of micro-conduit 40 or the destination site of the fluid. Various treatments of the fluid can take place within the micro-conduit 40 such as degassing, surfactant treatment, heating, incubating, mixing with reagents, and the like that can change the state of the fluid. Additionally, various membrane-based, enzymatic, potentiometry, amperometric, electrochemical, and immunological tests can be performed within the chambers 12 or micro-conduits 40.

The agent delivery device 10 of the present invention does not require separation and/or purification of fluids before performing assaying as in typical ELISA assays. All purification and preparation steps can occur within the device of the present invention (e.g., chromatography, primary incubation with antibody, enzymatic degradation, blood cell separation, blood cell lysis, and the like). Additionally, the agent delivery device 10 of the present invention is smaller than any other system that is utilized to perform conventional ELISA based assays. The present invention utilizes and requires significantly fewer quantities of antibodies, reagents, chromophores, samples, physical space, energy, and incubation time. The microscopic nature of the device of the present invention is more amenable to temperature regulation; thus, making the assays more precise and accurate, as well as reducing incubation periods (e.g., temperature control can be performed on the device to utilize integrated polysilicon heaters and thermocouples/thermistors). The size of the agent delivery device 10 also allows multiple assays to be run on a single dipstick-type device to provide color-coded testing results more useful for the layperson via in-home testing. Thus, multiple background, standards, sample duplicates, and the like can all be performed on a 1×1 inch device, which increases accuracy through statistical analysis. Alternatively, the device can be of a smaller size such as in the micro or nano range.

As mentioned above, the agent delivery device 10 of the present invention utilizes significantly less power than conventional microfluidic devices. It is compatible with standard CMOS fabrication and therefore the controlling circuitry can be integrated onto the substrate. It is calculated that less than 700 μW of power is necessary to achieve a pumping rate of 10 μL/min and that pumping rates of 100 μL/min are achievable with this design. Pumping volumes are accurate to within 5 nL volumes.

The agent delivery device 10 of the present invention has numerous embodiments. One embodiment is directed towards a micro-electro-mechanical system (MEMS) based agent delivery device 10 including at least one sampling chamber 12. The device can optionally include micro-conduits 40, sensor arrays 14, a microfluidic system 11, and an external monitoring system 44. The agent delivery device 10 can simply include one or multiple chambers 12 (i.e., sampling, reacting, and/or sensing). If there are multiple chambers 12, then they can be in communication with each other via micro-conduits 40. Alternatively, other embodiments are directed towards a device 10 including a sampling chamber connected to either reaction chambers 12 and/or sensor chambers 12 having sensor arrays 14. In any of the embodiments of the present invention, the system or device 10 can be placed on an attachable means such as a patch, Band-Aid, or other disposable sensor system. The device 10 can be placed directly onto the skin of a subject in order to obtain samples.

The chamber 12 (i.e., sampling, reacting, and/or sensing) of the present invention is generally illustrated in FIGS. 1 and 2. The chamber 12 provides for an area for placing the fluid, performing chemical reactions, sensing or detecting agents within the fluid, and/or collecting or storing the fluid. A simple one-step process can occur in one or more of the chambers 12. If numerous chambers 12 are utilized, these chambers 12 can perform required separations, measurements, and analyses of the fluid. For example, the chamber 12 can be used to lyse whole cells such as red blood cells by utilizing salts, chaotropes, heat, and any other similar reagents known to those of skill in the art. Additionally, certain chambers 12 can be utilized to contain just cells, while other chambers 12 contain only plasma therein. The actual structural components of the chambers 12 are outlined below and illustrated in the attached figures.

The chamber 12 can have various designs that have a flap or membrane covering the chamber 12 therein as well as configurations of supports 46 to act as stand-offs to prevent occlusion by the skin or to increase mixing and disrupt flow of the fluids therein. The supports 46 can vary in size and shape. For example, the bottom of the supports 46 can have a teardrop shape, oval shape, triangular shape, square, rectangular, cylindrical, and the like, while the top of the supports 46 is narrower or the same size and shape as the bottom portion thereof. The supports 46 also vary in size (i.e., volume) and shape in order to increase the volume capacity of the chamber 12.

The fluids within the agent delivery device 10 of the present invention primarily move via mechanisms including, but not limited to, capillary action, diffusion, microfluidic pumps, gravity, mechanical action, peristaltic action, pneumatic action, and any other similar mechanism known to those of skill in the art. The fluids can initially diffuse through membranes located on the device of the present invention and into various chambers 12. In other embodiments, there is no movement through a membrane.

The fluids move from chamber 12 to chamber 12 and within micro-conduits 40. Alternatively, active mechanical pressure induced by microfluidic pumps can aid in the movement of the fluids. For instance, positive or negative pressure on a membrane flap can move the fluids or active mechanical movement of micro-pumps 47 or actuators 30 can provide enough force to drive the fluids.

The microconduits 40 can be made of numerous materials as listed above. Additionally, the microconduits 40 can contain within the liner of the tube, placed in the tube or within the tube materials itself, various chemicals or reagents. The chemicals or reagents that are contained within the micro-conduits 40 or are impregnated within the micro-conduits 40 vary according to desired outcomes and reactions. For instance, the micro-conduits 40 can be coated with heparin to prevent clotting of blood, any surfactant to prevent bubbling of the fluid sample, charcoal to separate steroids, and any other similar substances known to those of skill in the art. Moreover, the micro-conduits 40 can be used to perform various treatments or reactions so that as the fluid sample travels along the micro-conduits 40, the reaction or treatment occurs and thus by the time the fluid sample reaches a designated chamber 12 or other location, the reaction or treatment is finished.

As discussed above, the agent delivery device 10 of the present invention can also include a microfluidic system 11 that aides in the quantitative and/or qualitative determination of the fluid samples. The microfluidic system 11 includes various components including, but not limited to, microfluidic pumps 47′, microfluidic devices additional chambers 12, microfluidic valves 50, microfluidic actuators 30, DNA chips, ports, micro-conduits or tubes 40, electrodes, and deflectable membranes made of materials such as glass, plastic, rubber, and any other similar materials known to those of skill in the art. A more detailed description of the microfluidic system is set forth in PCT/US01/27340, filed Aug. 31, 2001, which is incorporated herein by reference.

The microfluidic system 11 includes microfluidic actuators 30, which are the driving mechanism behind various components of the microfluidic system 11. The micro-fluidic valves 50 have various pressures and temperatures required for their actuation. The microfluidic pump 47′ is selectively controlled and actuated through an integrated CMOS circuit or computer control, which controls actuation timing, electrical current, and heat generation/dissipation requirements for actuation.

Integration of control circuitry is important for the reduced power requirements of the present invention. Closed loop feedback provides the basis of automated adjustment of circuitry within the micro-actuator 30.

The actuator 30 includes a closed cavity 52, flexible mechanism 34, and expanding mechanism 32. Fabrication of actuators microfluidic 30 is accomplished by generating electron-beam and/or optical masks from CAD designs of the micro-fluidic system. Then, using solid-state mass production techniques, silicon wafers are fabricated and the flexible mechanisms 34 for the microfluidic actuators 30 are subsequently placed on the chips.

In the microfluidic system 11 without integrated circuitry, the control circuitry is produced on external breadboards and/or printed circuit boards. In this manner, the circuitry is easily, quickly, and inexpensively optimized prior to miniaturization and incorporation as CMOS circuitry on-chip that can be controlled manually, or through the use of a computer with digital and analog output. Optimized CMOS circuitry, modeled utilizing CAD solid-state MEMS and CMOS design and simulation tools, is integrated into the active device making it a stand-alone functional unit.

Using an arbitrary waveform generator, and/or computer controlled digital-to-analog (d/a) and analog-to-digital (a/d) PCI computer cards (for example, the PCIM1016XH, National Instruments) the optimal operating parameters (i.e., stimulatory waveform patterns) are configured to generate peristaltic pumping action.

Electronic control of the microfluidic actuators 30 is optimized to maximize flow rates, maximize pressure head, and minimize power utilization and heat generation. Another parameter that is evaluated includes the temperature profile of the medium being pumped. To minimize power consumption and heat generation, a resistor-capacitor circuit is utilized to exponentially decrease the voltage of the sustained pulse. Further, integrated circuitry initiation and clocking of the circuitry provide control of the second-generation actuators.

An e-prom can also be included on-chip to provide digital compensation of resistors and capacitors to compensate for process variations and, therefore, improve the process yield. Electrical access/test pads are designed into the chips to allow for the testing of internal nodes of the circuits.

The flexible mechanism 34 deflects upon the application of pressure thereto. In one embodiment, the flexible mechanism 34 is screen-printed over the expanding mechanism 32 utilizing an automated screen-printing device, a New Long LS-15TV screen-printing system. The flexible mechanism 34 is very elastic and expands many times its initial volume as the expanding mechanism 32 under the flexible mechanism 34 is vaporized. Due to the large deflection, it is possible to completely occlude a micro-conduit 40 with this flexible mechanism 34, hence providing the functionality of an electrically actuated microfluidic valve 50. The present invention can also apply the flexible mechanism 34 with syringe or pipette devices or spin coat it on the entire wafer. Photo curable membrane can also be used to pattern the flexible mechanism 34 on the wafer.

A wide variety of commercially available polymers can be utilized as the flexible mechanism 34, including, but not limited to: Polyurethane, PVC, and silicone rubber. The actuator flexible mechanism 34 must possess elastomeric properties, and must adhere well to the silicon or other substrate surface. A material with excellent adhesion to the surface, as well as appropriate physical properties, is silicone rubber.

In an embodiment of the microfluidic system 11, the flexible mechanism 34 is made of silicone rubber. The silicone rubber can be dispensed utilizing automated dispensing equipment, or can be screen-printed directly upon the silicon wafer. Screen-printing methods have the advantage that the entire wafer, containing hundreds of pump and valve actuators, can be produced at once. By varying the amount of solvent in the polymer, such as silicone rubber, the flexible mechanism 34 thickness and its resulting physical force characteristics can be precisely controlled.

The flexible mechanism 34 can serve the dual purpose of actuation as well as serving as the bonding material used to attach the liquid flow channels to the silicon chip containing the actuators. By covering the entire area of the chip with the flexible mechanism 34, with the exception of the sensing regions and the bonding pads, the glass or plastic channels can be “glued” to the actuator containing silicon chip. This method provides additional anchoring and strength to the actuation flexible mechanism 34, and allows the actuation area to encompass the entire actuation chamber. The only drawback to this method is potential protein and/or steroid adsorption onto the micro-conduits 40. However, with proper flexible mechanism 34 selection and chemical treatment, molecular adsorption can be minimized, or a second, thin, inert layer can be used to coat the flexible mechanism 34.

The expanding mechanism 32 selectively expands the cavity defined by the flexible mechanism 34 thereof and thereby selectively flexes the flexible mechanism 34. The expanding mechanism 32 can be made of various materials. In one embodiment, the expanding mechanism 32 is a hydrogel material, which contains a large amount of water or other hydrocarbon medium, which is vaporized by the underlying heating mechanism 36. In this embodiment, the volume of hydrogel needed to produce the desired actuation and pressure for the flexible mechanism 34 is approximately 33 pL. With this design, approximately 97% of the energy generated by the heating mechanism 36 is transferred into the hydrogel for vaporization.

A practical technique for the microfluidic pumping of moderate volumes of liquid is through the use of peristaltic pumping utilizing pneumatic actuation. The integrated microfluidic pumping system 11 of the present invention is designed to sample small amounts of interstitial fluid from the body on a continuous basis. In order to analyze the microscopic volumes, silicon micro-machining methods and recent improvements in membrane deposition technologies are utilized to produce a microscopic test chamber 60 on the order of 50 nL in volume, roughly 3-4 orders of magnitude less volume than current systems. In addition to the improved response time, the reduction to microscopic volumes allows the use of very small amounts of calibration solution to effect calibration and rinsing, hence reducing the overall size of the package. In some systems the calibration solutions are a significant portion of the entire package (MALINKRODT MEDICAL/IL) where, even though miniature sensors are used, liters of calibration solutions are necessary.

In one embodiment, the microfluidic pump 47′ design is based upon electrically activated pneumatic actuation of a micro-screen printed silicon rubber membrane. Generally, the pump includes the microfluidic actuator 30 including a closed cavity 52, flexible mechanism 34 defining a wall of the closed cavity 52, and expanding mechanism 32 disposed within the closed cavity. The flexible mechanism 34 deflects upon the application of pressure thereto and the expanding mechanism 32 selectively expands the cavity and thus flexible mechanism 34 and thereby selectively flexes the expanding mechanism 32.

The microfluidic actuator 30 is based upon electrically activated pneumatic actuation of a micro-screen-printed or casted flexible mechanism 34. The peristaltic pump generally includes three actuators 30 placed in series wherein each actuator 30 creates a pulse once it is activated. By working in tandem, the actuators 30 peristaltically pump fluids. The optimal firing order and timing for each actuator 30 depends upon the requirements for the system 11 and are under digital control to create the peristaltic pumping action. The advantage of pneumatic actuation is that large deflections can be achieved for the flexible mechanism 34. To actuate the flexible mechanism 34, a vaporizable fluid is heated and converted into vapor to provide the driving force.

Utilizing an integrated heating mechanism 36, the expanding mechanism 32 is vaporized under the flexible mechanism 34 to provide the pneumatic actuation. This actuation occurs without the requirement of utilizing external pressurized gas.

The liquid or gaseous fluid being pumped serves the purpose of acting as a heat sink to condense the vapor back to liquid and hence return the flexible mechanism 34 to its relaxed state when the heating mechanism 36 is inactivated. A temperature sensor 38 is integrated adjacent to the actuator to monitor the temperature of the microfluidic integrated heating mechanism 36 and hence, expanding mechanism 32.

Once the heating mechanism 36 is activated, vaporization of the expanding mechanism 32 takes place. The expanding mechanism 32 component imposes a pressure upon the flexible mechanism 34 causing it to expand and be displaced above the heating mechanism 36 and reduces the volume of the chamber. This methodology can be utilized to displace fluid between the flexible mechanism 34 and the walls of the chamber (pumping action), to occlude fluid flow through the chamber (valving action), to provide direct contact to the glass substrate to effect heat transfer, or to provide the driving force for locomotion of a physical device (i.e., as in a walking caterpillar and/or a swimming paramecium with a flapping flagella, in which case the glass chamber encompassing the microfluidic actuator 30 is not used).

In one embodiment, the temperature of the saturated liquid hydrogel, at 1 ATM, is assumed to be 100° C. The heat flux to the air, through the back of the heating mechanism 36, is calculated to be 1263 W/K-m². The total heat flux through the device is calculated to be 46,995 W/K-m² with a total flux from the heating mechanism 36 of 47,218 W/K-m² (i.e. 97% efficiency of focused heat transfer). In this embodiment, the temperature of the inactive state hydrogel varies between 86° C. and 94° C.

The temperature of the activated, vapor state hydrogel is approximately 120° C., which is the saturation temperature for steam at 2 ATM. The heat transfer coefficient for convection can be calculated directly from the thermal conductivity.

The heat flux to the air through the back of the heating mechanism 36 is 2818 W/K-m². The heat flux through the device is 21,352 W/K-m² with a total flux from the heating mechanism 36 of 24,170 W/K-m². When the aqueous component of the hydrogel is completely in the vapor state, there is no fluid in the channel and the thin film of solution between the flexible mechanism 34 and the glass is approximately at 60° C. These values and calculations vary according to the type of actuator, valve, pump, and micro device being used.

In an embodiment of the present invention, the volume of the expanding mechanism 32, in this case, liquid hydrogel, is determined based on the volume of vapor needed to expand the flexible mechanism 34 completely at 2 ATM using the ideal gas law. This assumption is valid because the temperatures and pressures are moderate. The volume of liquid hydrogel necessary to achieve this volume of gas at this pressure, assuming the hydrogel is 10% water and all of the water is completely evaporated, is 0.033 nL. Cylindrically shaped sections of hydrogel are utilized within the microfluidic actuator 30. This shape has been chosen to optimize encapsulation by the actuator flexible mechanism 34. The cylinders have either a diameter of approximately 140 μm and a height of 2.14 μm, or a diameter of 280 μm with a height of 0.54 μm (identical volumes, different orientation to the heating element). Of course, the shapes and volumes vary according to the type of expanding mechanism 32 being used. For example, photocurable liquid hydrogels have different parameters.

The heating mechanism 36 is poly-silicon, but can be any similar material or mechanism, such as direct metals, known to those of skill in the art. Because of its high thermal conductivity, the silicon substrate acts as a heat sink. To reduce thermal conduction to the silicon substrate, a window in the silicon, located beneath the heating mechanism 36, provides the expanding mechanism 32 with an isolated platform. This window is only slightly larger than the heating mechanism 36 to maintain some thermal conduction to the substrate. After the microfluidic actuator 30 is energized, thermal conduction to the silicon provides decreased time to condense the liquid in the expanding mechanism. This decreases constriction time and provides improved pumping rates. If the window is significantly larger than the microfluidic actuator 30, there is no heat conduction path to the substrate, hence increasing condensation time and decreasing the maximal flow rate.

A polymeric hydrogel (or hydrocarbon) can be utilized to provide a physically supportive structure that withstands the application of flexible mechanism 34 as well as to provide the aqueous component required for actuation. Several commercially available materials meet these requirements. A hydrogel is selected that contains approximately 30% aqueous component that vaporizes near 100° C. Several materials have been identified, each of which is suitable in this application, including, but not limited to, hydroxyethylmethacrylate (HEMA) and polyvinylpyrrolidone (PVP).

Additionally, hydrocarbons can be used since they possess lower boiling points than aqueous hydrogels, and therefore require less power to effect pneumatic actuation. Dispensing hydrogel (or hydrocarbon) into the desired location is accomplished utilizing one of three methods. First, a promising method for patterning the hydrogel is to utilize a photopatternable-crosslinking hydrogel. The hydrogel is cross-linked by incorporating an UV photo-initiator polymerizing agent within the hydrogel that cross-links when exposed to UV radiation. Using this technique, the hydrogel is evenly spun on the entire wafer using standard semiconductor processing techniques. A photographic mask is then placed over the wafer, followed by exposure to UV light. After the cross-linking reaction is completed, excess (non-cross-linked hydrogel) is washed from the surface.

The second method involves dispensing liquid hydrogel into well rings created around the poly-silicon heating mechanism 36. These wells have the ability to retain a liquid in a highly controlled manner. Two photopatternable polymers have been utilized to create microscopic well-ring structures, SU-8 and a photopatternable polyimide. These well rings can be produced in any height from 2 μm to 50 μm, sufficient to contain the liquid hydrogel. Once the hydrogel solidifies, flexible mechanisms 34 can be deposited over them. This can be accomplished in an automated manner utilizing commercially available dispensing equipment.

In a third alternate method, a pre-solidified hydrogel is used that has been cut into the desire size and shape. This is facilitated by extruding the hydrogel in the desired radius and slicing it with a microtome to the desired height, or by spinning the hydrogel to the desired thickness and cutting it into cylinders of the desired radius. Utilizing micromanipulators, the patterned gel is placed in the desired area. This process can also be automated.

It is assumed that the temperature on both sides of the SiO₂ that encapsulates the heating mechanism 36 is constant, and that heat flux in each direction is dependent upon the heating mechanism 36 temperature and both sides are resistant to heat flow either through the device or to an air pocket on the heating mechanism 36 backside. Steady-state heat flow through the entire actuator, for the fully actuated state, the intermediate state, and the resting state are modeled. These data are calculated for the static case during which time no fluid flow is occurring (i.e. steady-state; the system is poised at 100° C., waiting to be initiated). The fluid temperature is greater for the contracted state since the liquid hydrogel conducts heat at a greater rate than vapor. Once fluid flow is initiated, the temperature of the solution is raised by only a few degrees Celsius.

A typical problem experienced with many microfluidic designs revolves around the methodology for mixing of solutions and reagents. The microfluidic pump 47′ design of the present invention provides mixing action in concert with the pumping action. To construct the microfluidic valves 50 and pumps 47′ in a manner compatible with the sensor technologies and to integrate the entire system on a single silicon chip, the pump is preferably fabricated using planar MEMS technologies that do not require special wafer bonding, although other methods of fabrication can also be used as are known to those of skill in the art.

For encapsulating a liquid within a silicone rubber membrane, micro-machining techniques, including wafer bonding of multiple chips, are used by others to create a cavity where the liquid is stored. This requires several machining steps to produce the actuator, reducing the overall yield of functional pumps and valves, and increasing the cost.

By properly placing the planar actuators within the fluidic channels, micro-pumps, fluidic multiplexers, and valves can be formed. CAD/CAM tools are used to design the photo-masks. This can be accomplished in conjunction with the design of the fluidic channels, ports, and test chambers.

The pneumatically actuated membrane is utilized to produce the microfluidic valves. The microfluidic actuator's silicone rubber membrane is very elastic and expands many times its initial volume as the liquid under the membrane is vaporized.

At least two techniques for the valving of solutions can be used. The first utilizes the flexible mechanism 34 actuation to completely fill a microfluidic channel when actuated, hence providing the functionality of an electrically actuated microscopic valve. The second utilizes the flexible mechanism 34 to occlude an orifice to block fluid flow.

The pneumatically actuated membrane is also utilized to produce the microfluidic pumps. The microfluidic actuator's flexible membrane 34 is very elastic and expands many times its initial volume as the liquid under the membrane is vaporized. The micro-conduits 40 are designed such that all media flow is in the laminar regime while minimizing fluid volume, dead volume, and residence time.

Further, the routing of the micro-conduits 40 is designed such that the required calibration and wash solutions can be routed into the sensing chamber 12. The micro-conduits 40 and sensor chamber 12 accommodate approximately 50 nL volumes of solution.

Once modeled and optimized, photomasks are created for the fluidic system. Valves at the various ports are optimally designed to start and stop the flow of the various calibration and wash solutions.

In one embodiment, the integration of a sampling system or microfluidic system 11 to the agent delivery device 10 allows transdermal-sampling techniques for the acquisition of interstitial fluids. This sampling chamber 12 has a maximized surface area within the confines of the agent delivery device 10 and an extremely minute volume to reduce the required sample volume and to decrease the sampling time. This chamber 12 is micro-machined into the backside of the glass fluidic channel chip.

For mobile applications, automated control of the pumps, valves, and sensors is required to continuously monitor and calibrate the microscopic “lab-on-a-chip” devices. Using integrated electronics, the sensor arrays 14 can be calibrated on a regular basis in an automated manor that is transparent to the user, ensuring accuracy of the data obtained. The sensing system also requires integrated circuitry to buffer the signals, reduce noise, transduce the chemical concentrations into electronic signals, and analyze the signals, allowing untrained personnel to utilize the device.

Another application for integrated circuitry is for the telemetric communication of the device with a base unit, which can then relay the information to a remote location. Moreover, the circuitry can perform closed-loop feedback control for biological applications. For example, closed-loop feedback control can be used to inject insulin into an individual when the transdermal sensor system detects hyperglycemic levels of glucose in the transdermally sampled interstitial fluid, thereby maintaining euglycemia.

The sensor arrays 14 are fabricated in a three-mask process with two metal layers, silver and platinum. Since these metals are difficult to etch using wet chemistry, a resist lift-off process was used to pattern them. This provided an additional advantage in allowing the use of layered materials in a metal structure to modify electrode properties and still allowed for patterning to occur in one step.

Additionally, other sensor array 14 conformations can be produced in accordance with the present invention, each with differing transduction, and membrane encapsulation properties. These designs incorporate rectangular, circular, and concentric circle shaped electrodes.

In any embodiment, the microfluidic valves 50 of the present invention utilize an actuating mechanism to occlude a micro-conduit 40 and thereby decreasing or preventing fluid flow. The ability to occlude is selective, in that the valve can effectively open and close a passageway of the micro-conduit 40. The microfluidic actuators 30 are the driving mechanism behind the microfluidic valves 50 of the present invention.

For a mono-stable microfluidic valve 50, it is assumed that the temperature on both sides of the Si0₂ that encapsulates the heating mechanism 36 is constant, and that heat flux in each direction is dependent upon the heating mechanism 36 temperature and the general resistance to heat flows either through the device or to the air from the backside. In order to isolate the heater, a cavity is etched in the backside of the wafer, providing thermal isolation. The mono-stable microfluidic valve 50 requires continuous power to maintain a closed-stated position. Utilizing the heating mechanism 36, an expanding mechanism 32 is vaporized under the encapsulating flexible mechanism 34 thereby providing the pneumatic driving force required for expanding the flexible mechanism 34 and hence occluding the micro-conduit 40. The mono-stable, normally open microfluidic valve 50 utilizes a single actuator to effectively actuate the valve. As the hydrogel is expanded, the silicone rubber of the actuator completely occludes the micro-conduit 40 to effect valving of the solution. While the normally open microfluidic valve 50 is less complicated to construct, it requires continuous power or pulsed power to keep the valve closed.

A bi-stable microfluidic valve 50 is also capable of being utilized. The bi-stable microfluidic valve 50 is designed that utilizes lower power consumption and a wax material to provide passively open and passively closed functionality, i.e. bi-stability. Thus, power is only required to transition from one state to the other. The bi-stable valve design is based upon the utilization of a moderate melting point solid, such as paraffin wax, which possesses a melting point between 50° C. and 70° C.

The bi-stable microfluidic valve 50 similarly utilizes actuating mechanisms to occlude the micro-conduit 40. The mono-stable microfluidic valve 50 can only provide the functionality of a normally open valve. During the period that the valve must be maintained in a closed position, continuous power must be applied. The bi-stable microfluidic valve 50 utilizes microfluidic actuators 30 to provide both zero-power open and closed functionality.

The bi-stable microfluidic valve 50 utilizes a total of three actuators 30. Any number of actuators 30 can be used without departing from the spirit of the present invention. Two actuating mechanisms are physically connected by a micro-conduit 40 formed under the membrane and are filled with a low melting point solid such as paraffin wax as opposed to an aqueous hydrogel (see above for mono-stable actuation). The third is a standard design micro-actuator filled with an aqueous hydrogel connected by the expansion chamber to the middle wax filled actuator. The first two actuators 30 are activated causing the wax to melt. The third, standard, micro-actuator is then activated, providing pneumatic force on the wax containing actuators, causing the orifice containing chamber to close. The wax is then allowed to solidify. Again, the advantage of this valve is that it requires power only to transform from the stable open to the stable closed state.

In the open state, medium in the channel readily flows. To switch from the open state to the closed state, the wax is melted and the pneumatic actuator 30 on the right is expanded. This creates pressure outside the middle actuator, forcing the paraffin into the smaller left chamber, expanding the membrane, thereby blocking fluid flow. The wax is allowed to solidify, after which the power can be removed from the actuator providing the driving force pressure, resulting in an electrically passive closed state. To transition from the closed state to the open state, the wax is melted and membrane tension forces the wax from the small left chamber back into the middle chamber. The micro-valve design provides bi-stable functionality, which only requires power to switch between each state, but is completely passive once in either the open or closed position.

The use of polydimethylsiloxane (PDMS) in multiple layers to directly produce the three-dimensional structures of the microfluidic system is a technique well suited to mass production. This technique has the advantages of allowing an entire wafer of chips to be packaged simultaneously and of being compatible with integrated circuitry. This process is fairly complex, requiring multiple photo patterning of the devices and the application of a top layer to complete the structure. Despite the manufacturing challenges, this method is capable of creating three-dimensional microfluidic systems. PDMS has the following properties: low glass transition temperature, low surface energy, high permeability of gases good insulating properties, and very good thermal stability. The properties of PDMS can be altered such as to convert the surface from hydrophobic to hydrophilic. This can be accomplished by numerous methods known to those of skill in the art including, but not limited to, oxygen plasma treatment, hot acid treatment, surface coating with polyurethane, and surfactant treatment.

The sensors 14 of the present invention include at least one amperometric sensor, and at least one potentiometric sensor. The sensors of the present invention can detect neuronal action potentials and the resulting release of neurotransmitting and/or hormones. The sensors can also detect the diffusion, dispersion, degradation, and re-uptake of neurotransmitters, hormones AND/OR other cellular metabolites. Examples of such sensors 14 are known to those of skill in the art and more specifically, sensors are disclosed in co-pending U.S. patent application Ser. No. 10/111,964, filed May 2, 2002.

Coulometry is the determination of charge passed or projected to pass during complete or nearly complete electrolysis of an analyte, either directly on the electrode or through one or more electron transfer agents. The current, and therefore analyte concentration, is determined by measurement of charge passed during partial or nearly complete electrolysis of the analyte or, more often, by multiple measurements during the electrolysis of a decaying current and elapsed time. Once the hydration shell has been established around the electrode, the decaying current results from the decline in the local concentration of the electrolyzed species caused by the electrolysis. A compound is immobilized on a surface 26 when it is physically entrapped on or chemically bound to the surface.

Electrochemical detection, specifically amperometry, has been used in the past in relatively unsophisticated applications, for example, detecting and quantifying eluted molecules at the end of chromatographic columns (Kissinger et al, 1984).

The main limitations of amperometry are its low specificity and sensitivity. The present invention takes advantage of this technique's speed and overcomes its limited specificity and sensitivity. First, to enable the amperometric sensors 20 to detect multiple neurotransmitters independently, the sensors employ two particular forms of amperometry; cyclic and constant voltage voltammetry. Second, utilizing a micro-screen printing device, such as a New Long LS-15TV, several different selectivity membranes can be applied over the individual sensors to eliminate background measurement of unwanted compounds (such as ascorbic acid) and impart specificity onto the microscopic electrodes including the sensor (Goldberg et al, 1994). Finally, by encapsulating the multi-site sensor array 14 leads with silicon nitride, which is a substrate that neurons can be made to readily attach, the sensor array is in very close apposition to the secreting neurons allowing measurement of the relatively high neurotransmitter concentrations in the immediate vicinity of the axon, prior to degradation, dilution, dispersion, and re-uptake.

An amperometric process, cyclic voltammetry, is a technique whereby a cyclically repeated triangular waveform of potential is applied between the working and counter electrodes. Individual analytes, such as neurotransmitters, have characteristic oxidation and reduction potentials based on their chemical moieties (Adams, 1969; Dryhurst et al, 1982). When the voltage between the electrodes reaches the oxidation potential of a particular neurotransmitter that molecule oxidizes. Oxidation is a process whereby an electron is stripped from the molecule. The counter electrode absorbs the oxidatively produced electrons, effectively transducing chemistry into electricity. The flow of electrons per unit of time is current, which is proportional to the number of molecules being oxidized. The voltage at which this oxidatively produced current is obtained provides information useful for identifying the analyte such as neurotransmitter, hormone or cellular metabolite being measured (Dryhurst et al, 1982; Baizer et al, 1973).

Other embodiments of the sensor array can include, but is not limited to, additional components such as various separating and purifying mechanisms, heating elements to aid in the lysis of cells, adding and mixing mechanisms, and degassing mechanisms to remove air bubbles. Moreover, various agents can be added to the present invention including, but not limited to, surfactants, primary antibodies to start ELISA reactions, other enzymes to start desired reactions, color reporters (HRP), luminescent agents, or other indicators, and any other chemicals or substances known to those of skill in the art.

In another embodiment of the present invention, the device can be used in conjunction with a hand-held reader for electronically timing the reaction rates and provide digital read-out to automate the measurement process so as to eliminate the need for trained personnel. In this embodiment, the device includes a disposable cartridge containing the enzyme chemistry reagents, detection chambers, and microconduits, a reader containing the sensors, actuators and controlling electronics, and a hand-held read-out system.

The hand-held read out system is usable by both the clinician as well as the patient themselves. It can be designed and developed for use with the device of the present invention. The readout device can be designed as a “hand-held” readout and controlling instrument (RCI) utilizing commercially available Palm or Windows CE hand-held computers. The RCI can be utilized to provide an ergonomic display of sensor and calibration data as well as to monitor trends in the patient. The RCI can control the actuator timing to obtain more or less frequent samples and/or calibrations in a given time period. The RCI unit is also responsible for sensor data conversion utilizing the calibration parameters.

On the chip-based sensor unit, the data is stored in a digital manner until it is ready to be read by the RCI. The RCI accepts a stream of data from the sensor unit and display it in one of two different configurations. The first software implementation in the RCI is for the patient that can display subjective data. In other words, if concentrations are in a high, normal, or low range, then trend analysis providing simple exposed/not-exposed information to the patient. The second version can be utilized by the clinician or trained personnel, who can receive a readout that displays quantitative data from the sensor array and allows data output for use in any standard database or graphing program. In addition, the RCI allows the clinician to control-the acquisition device, including sampling frequency, calibration frequency, alarm settings, etc. Numerical concentration levels and trends can be displayed on a hand-held computer or PDA. Furthermore, compatible integration into a Medical database for the individual can take place.

The present invention provides a transdermal glucose monitor with a Bluetooth™ transponder for wireless technology for the purpose of transmitting glucose data from the patch to a remote computer. Although there are a wide variety of alternative wireless technologies that could be employed, Bluetooth™ was chosen for a number of reasons.

Bluetooth™ wireless technology is specifically designed for short-range (nominally 10 meters) communications; one result of this design is very low power consumption, making the technology well suited for use with small, portable personal devices that typically are powered by batteries. A typical Bluetooth™ device draws less than 0.3 mA in standby mode and an average of 5 mA in raw data mode. Bluetooth™ was designed to be simple to implement, have low power consumption and be relatively inexpensive. There is no need for a line of sight between the Bluetooth™ transponder and receiver since Bluetooth™ uses a radio link for communications. These characteristics make Bluetooth™ well suited for use in medical applications such as physician tools, diagnostic instruments and telemedicine.

A Bluetooth™ module consists primarily of three functional blocks, an analog 2.4 GHz Bluetooth™ RF transceiver unit, a baseband link controller unit, and a support unit for link management and host controller interface (HCI) functions. Bluetooth™ uses Frequency Hopping Spread Spectrum (FHSS) technology (1600 hops/second) to increase the reliability of the communication channel. The signal hops among 79 frequencies at 1 MHz intervals to give a high degree of interference immunity. Bluetooth™ devices form networks called Personal Area Networks (PANs) or piconets. Up to seven simultaneous connections can be established and maintained in a piconet. The device that establishes and controls the piconet is called a master and all other seven devices in the piconet are called slaves. These piconets are established dynamically and automatically as Bluetooth™ devices enter and leave the radio proximity. This allows many different devices to be used by many different users in a dynamic environment. Each piconet uses a slow hopping frequency with a pattern determined by the master. The timing of the network is also done by the master with the slaves synchronizing to the master's clock. Using this methodology, Bluetooth™ devices are capable of 723.2 kbps, which is more than sufficient for the proposed glucose monitor.

Bluetooth™ technology can be either built into an electronic monitoring device or used as an adaptor that plugs into these devices. The Bluetooth™ device contains a circuit board, power supply, Bluetooth™ core chip, Bluetooth™ RF (radio) module, interface (USB or RS232), PCM chip and audio interface for audio interface and connector for external antenna.

There are three ways of implementing Bluetooth™ wireless technology into an end product. The first is by using a Bluetooth™ module. Although it is a very expensive and inflexible method, this is the easiest method that offers fastest time-to-market solutions. The second method is to use a pre-qualified Bluetooth™ chipset. The off-the-shelf items are available in the market for integration into the system level of the product. The third method is to directly incorporate Bluetooth™ circuitry directly into the product being developed. The IP for directly incorporating Bluetooth™ into a product can be purchased from providers such as Newlogic, Ericsson or ParthusCeva.

Develop the software architecture: There are three aspects of the software system that are required: the overall software architecture, the interface between the patch and the remote computer, and the user interface. AST plans to use modern (object oriented) methods for developing all aspects of the device's software.

The software architecture describes the relationship of the system's data objects with other data objects and with external systems. The system has two data producers: the patch and the user input, and one data consumers: the local display of data. Since each of these data objects can act relatively independently, the number and complexity of the interactions between the system's data objects are likely to be minimal.

To be able to operate within this type of environment, one must either employ a common interface or employ an interface that works with a defined subset of external systems.

SQL Server CE is a compact database for rapidly developing applications that extends enterprise data management capabilities to mobile devices. SQL Server CE makes it easy to develop mobile applications by supporting the industry-standard Structured Query Language (SQL) syntax. SQL Server CE also provides a range of data types and supports 128-bit encryption on the device for database file security.

The SQL Server CE engine exposes a broad set of relational database features while maintaining a compact footprint that enables applications using this engine to be deployed to a wide variety of PocketPC devices. The programming and operational model, which is consistent with the rest of the SQL Server family, facilitates the development of new applications and integration with existing systems. SQL Server CE is easily integrated with the Microsoft .NET Compact Framework by means of Microsoft Visual Studio .NET, thereby simplifying database application development. This allows mobile application developers to build highly extensible applications with offline data management capability for disconnected scenarios.

This is a key feature not present in existing mobile databases. SQL Server CE is particularly well suited for mobile and wireless environments as it has methods for remote data access and ensuring merge replication with SQL Server databases. Remote data access exposes data in SQL Server databases through remote execution of Transact-SQL statements and providing the ability to pull record sets to the client device for updating. SQL Server CE provides the ability to synchronize through merge replication. These data access technologies take advantage of Internet standards, including HTTP Secure Sockets Layer (SSL) encryption, through integration with Internet Information Services (IIS). This approach ensures data can be accessed reliably and flexibly, even through firewalls. These are important capabilities as MS SQL server is one of the three most commonly deployed databases and IIS is one of the two most commonly deployed web servers.

Later versions of the software can employ the Extensible Markup Language (XML) for data interactions with external systems. XML is a markup language for documents containing structured information. Structured information contains both content and an indication of the role that content plays. A markup language is a mechanism to identify structures in a document. The XML specification defines a standard way to add markup to documents. XML is an international standard and most all modern computers provide the ability to create and parse XML documents. By employing an XML-based interface, all computers are able to interact with the data provided by the PDA.

Although the shift to XML might seem like a radical departure from the SQL method described previously, it is actually an enhancement to the proposed system, not a replacement for it. This is due to the fact that both SQL Server CE and Microsoft Visual Studio .NET, the development environment of choice for mobile SQL Server CE applications, provide extensive support for building and deploying web-based XML applications. The integration of this capability can provide the broadest possible base of support for the system.

Design the user interface: Possibly the most important aspect of the software design is the graphical user interface (GUI). Aspects of this task include defining the users' interaction with the system, defining the means for inputting data into the system, and defining the data presented to the user and the format in which it is presented.

The patient has several modes of interaction with the device. Representative interactions include, but are not limited to, inputting relevant therapeutic information into the system, recalling historical data for analysis and study, and uploading data to a centralized system.

As shown above, several of the physician's interactions with the device include the entering of data. Since the core of the device is a PDA, the most obvious choice of methods for inputting this data is via on-screen buttons and/or written notes. A suite of buttons and/or free-form text fields was carefully designed to provide the physician with the greatest possible degree of flexibility while minimizing the effort to input the data. Additionally, the device can use voice input. At the least, voice input could be used by the physician to store examination notes. With the use of voice recognition, it is possible to eliminate the need for manual data input.

The GUI can be developed in such a manner as to make the device as easy to use as possible. This means that each screen has a single purpose, such as data entry, viewing results, etc., and that the most obvious controls can sequence through the screens in a typical fashion. To provide the physician with full control, all system functions can be available (probably through a menu system), though the ones that are infrequently used can require one or two levels of menu navigation to reach.

The device was developed using a MS CE.NET compliant PocketPC. The two primary reasons for choosing this platform are the wide availability of such devices with CF ports and the ease of graphically developing GUI's using Microsoft Visual Studio NET. By using a graphical design paradigm, the software developer can more easily develop systems that are ergonomically sound and visually pleasing.

Develop a stand-alone version of the software: The distinguishing feature of the proposed system is its use of an industry standard, relational database as the core of the software aspect of the product. This contrasts with all other PDA-based programs for managing diabetes that employ proprietary, flat-file systems. Since the database is the core of the software program, the first step in developing the application is developing the database schema.

A schema is the logical structure of the database, i.e., it defines the relationships between each of the data objects contained within the database. The figure shows a preliminary sketch of a schema for this project:

The schema focuses solely on the dietary logbook aspect of the project. Additional tables for storing personal information, sensor readings, and other user supplied data can be added to this schema when development commences. There are several noteworthy features of this schema: 1. Data items are never removed from the database, instead, they are marked as being inactive. This guarantees that data analyses performed in the future can always return valid data; 2. The grouping of food items into groups greatly facilitates searching for items. This is supported by the use of many-to-many relationships that helps ensure data normalization; and 3. Since the data is being stored in a relational database, searches can be performed using any combination of criteria, thereby making it possible to quickly locate data items of interest.

The next aspect of the software development is the implementation of the GUI (see Task 5). To facilitate development, the program was developed using Microsoft Visual Studio .NET 2003, which has built-in support for PocketPC development. The tools provided permit developing applications for PocketPC's in the exact same manner as for desktop systems. Visual Studio also facilitates the development of database applications through the use of SQL-specific data objects and methods.

To facilitate usage of this system, it was necessary to populate the database, especially the Food and Group tables, with typical foodstuffs so users can immediately start entering there consumption data without first having to populate these tables. To perform this subtask, a database was identified with the necessary information that is in the public domain and can import the data into the database.

The present invention can be used to detect the presence of various agents and substances as described above. Additionally, the present invention can detect and determine whether exposure to an agent has occurred through the detection of antibody presence and levels thereof. Additionally, the present invention can be used to detect the biological effect of exposure to such various agents and substances as described above.

The device of the present invention is capable of directly determining the presence of an agent, the presence of a reaction to an agent, and providing a differential analysis of an agent level and correspondingly responding to the analysis. For example, the device is capable of providing a differential blood ChE analysis. Thus, the device provides a full analysis of a patient's cholinesterase levels using a single drop of blood obtained from finger prick sampling. The device is automated such that minimally trained personnel can utilize it, and provides results in approximately 5 minutes or less. Additionally, the device specifically can monitor acetylcholinesterase (AChE) levels within red blood cells (RBCS) and butyrylcholinesterase (BuChE) levels within plasma. The device is capable of performing these tests within a few minutes and with less than a 5 μl sample of capillary blood.

Lyophilized enzyme detection chemistries can be incorporated into the device in the form of membranes on the assay pads. The membrane coated assay pads undergo calorimetric changes in response to analyte concentration. The device incorporates various microscopic, solid-state, photo diode sensors that can be plugged into a hand-held or laptop computer to objectively monitor the assay results. Alternatively, potentiometric and/or amperometric sensors can be employed. Thereby, simple assays or complex enzyme or antibody assays can be utilized.

The device of the present invention can be used in a variety of settings including, but not limited to, health clinics, emergency rooms, hospitals, clinical settings, home health care market, offices, work places, points of chemical exposure including possible terrorist attack sites such as in planes, trains, buildings, and any other similar settings requiring the monitoring or screening of individuals to determine and confirm exposure to various toxins and/or agents. Thus, the present invention is not meant to exclude any application outside of the medical field.

Furthermore, the present invention is well suited to test any subject including, but not limited to, employees, workers, athletes, EMS personnel, emergency first responders, and any other subject who is in need of administration of an agent for treatment of a disease or condition.

The present invention can be used to detect or treat any disease or condition. For example, the device of the present invention can be used to detect agents in order to diagnose diseases or detect the presence of toxins or pollutants. Further, the system of the present invention can be used to treat the detected disease. The following list is meant to include, but is not limited to conditions that can be treated, biological contaminants, chemical contaminants, environmental pollutants and toxins, effects of chemotherapy, levels of bilirubin, drug effectiveness, disease states, and the amount of an allergic reaction.

For example, the present invention can be use to treat diseases or conditions. Examples of such diseases include malaria, diabetes, infertility, substance addiction, dermal treatments, and other conditions as listed below.

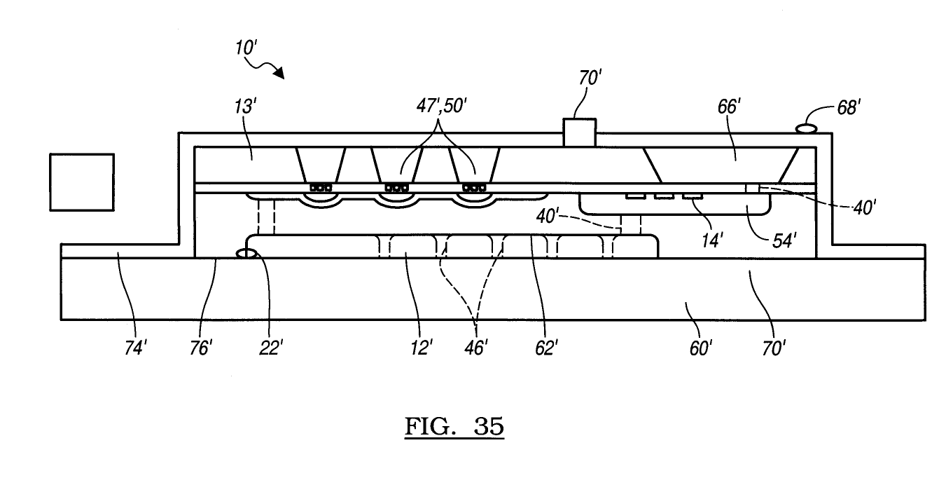

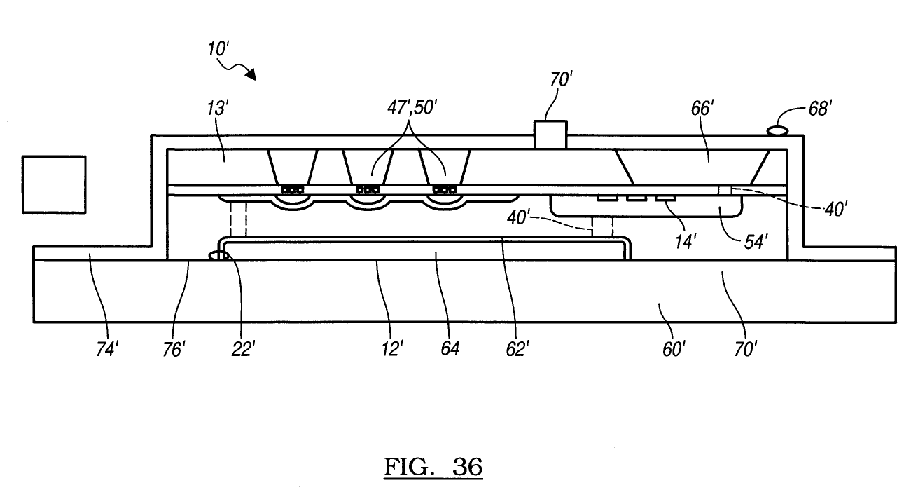

In a further embodiment, the agent delivery device 10 includes a body portion 13′ housing a transmembrane fluid capturing chamber 12′ for capturing interstitial fluid through a membrane 60′ and a testing chamber 54′ for detecting molecules in captured interstitial fluid, as shown generally in FIG. 35. The transmembrane fluid capturing chamber 12′ is also described as a membrane interface chamber 12′ because it is situated against and adjacent to a membrane 60′. The membrane 60′ can be skin, a membrane in vitro, or any suitable membrane in/on a body. The agent delivery device 10′ is small, on the order of a few square centimeters or less. The agent delivery device 10′ is manufactured essentially as described above, and integrates the circuitry, microfluidic devices, and other elements of the agent delivery device 10 as described above. The membrane interface chamber 12′ is made of material and manufactured as described for the chamber 12 above.

The membrane interface chamber 12′ can include an operatively attached electrode(s) 22′ for performing iontophoresis/electroporation in order to obtain interstitial fluid from the membrane 60′. Iontophoresis is a means of enhancing the flux of ionic compounds across a membrane through the application of an electric current. The top layer of the skin, the stratum corneum, is the main barrier to drug and molecular transport, however with the help of an electric current, molecules can pass through the skin easier. There are two principal mechanisms by which iontophoresis enhances molecular transport across the skin: (a) iontophoresis, in which a charged ion is repelled from an electrode of the same charge, and (b) electroosmosis, the convective movement of solvent that occurs through a charged “pore” in response to the preferential passage of counter-ions when the electric field is applied. Iontophoresis can also be operated in the reverse, wherein applying an electric current across the skin extracts a substance from beneath the skin. For larger molecules, and increased transport, electroporation uses short (100-300 ms) pulses of very high voltage (50-250V) to increase transdermal interstitial fluid transport. This method of drug delivery increases mass transport across the dermal membrane by several orders of magnitude. Electroporation efficiency is dependent on both the duration and amplitude of applied voltage. Short pulses between 4V and 15V have been shown to increase the epidermal conductance, but not the effective pore radii, while longer pulses (on the order of 50 min) have been demonstrated to increase pore radii. This method is compatible with larger molecule transport through the skin, at much higher rates, and it has been demonstrated that 40 Kda molecules can be transported through the skin with this method without any skin enhancers.

The membrane interface chamber 12′ includes a base 62′ to which supports 46′ can be attached, such as the posts described above which allow for mixing of fluid (either captured interstitial fluid or molecular fluid from a reservoir 72′) from the formation of eddies in the membrane interface chamber 12′. The base 62′ can also be covered by a separation membrane 64′ to maintain a gap or a distance between the base 62′ of the membrane interface chamber 12′ and the membrane 60′, as shown in FIG. 36.

The separation membrane 64′ can be any suitable membrane, for example an electrolyte polymer membrane. Polymer matrix electrolytes have been shown to be ideal for storage and delivery of molecules, such as lithium and lidocaine using iontophoresis. Polymer electrolytes are solid-like materials formed by dispersing a molecule/therapeutic, such as nicotine for cessation of smoking, in a high molecular weight polymer. In essence, the molecule is trapped within the polymer until the application of an electric current. Application of electric current, such as by electrodes, causes the porosity of the polymer to increase, hence providing controlled release of a molecule. This technology allows molecular concentrations of nicotine as high as 4M to be incorporated into the matrix. The use of polymer electrolytes to deliver molecules can simplify the agent delivery device considerably since it may eliminate the need for reservoirs and pumps. CMOS circuitry controls the amplitude and duration of the molecule transfer in order to deliver precise amounts of the desired molecule. This may also provide a secondary fail-safe mechanism in case of trauma to the agent delivery device 10′, or failure mode operation since transdermal delivery of the desired molecule can only occur when current is applied.

Polymer electrolytes are ionically conducting polymers that are composed essentially of solutions of ionic salts in heteropolymers, such as poly(ethylene oxide) (PEO). PEO is a semicrystalline solid with a high proportion of crystalline regions distributed in a continuous amorphous phase, which means the PEO is a solid at room temperature (tm=65° C. and Tg=−60° C., thus it has structural integrity) and the PEO chains in the amorphous regions have a sufficient degree of segmental mobility, permitting ion transport. The amount and state of amorphous regions of polymer is therefore crucial to its functioning as a polymer electrolyte, which can be altered by many factors, including the type and amount of added ions (including medicinal drugs) and the method by which the polymer electrolyte is formed.

As its low molecular weight analogs, the poly(ethylene glycol)s, the PEO has minimal adverse reactions to skin (skin irritation and sensitization), as well as a sufficient loading capacity of drug dose. Unlike its low molecular weight analog like poly(ethylene glycol), which tends to form liquid or semisolids, PEO forms a solid matrix. The drug delivery property of the polymer electrolyte film for iontophoresis is assessed by checking its AC impedance. PEO-salt complexes can be formed as soft, flexible films with a thickness that can vary from a few micrometers to about 100 micrometers. Previous studies showed that PEO can incorporate large concentrations (˜4M) of salt, making it eminently suitable as a matrix into which highly potent drugs may be incorporated.

The membrane interface chamber 12′ can be removably attached to the body portion 13′ so that it can be disposed of, for sterility issues, and making the testing chamber 54′ reusable. As shown in FIG. 37, the membrane interface chamber 12′ can be removably secured to the body portion 13′ through the use of a die-locker 78′ for locking the membrane interface chamber 12′ in place and a spring 80′ for releasing the membrane interface chamber 12′. Any other suitable lock and release mechanism can also be used.

The testing chamber 54′, having the properties of the sensing chamber 12 described above including various sensors (such as a sensor array 14′), is a housing in which a reaction(s) is performed on the captured interstitial fluid. The testing chamber 54′ is operatively connected to the membrane interface chamber 12′ through at least one micro-conduit 40′. The captured interstitial fluid in the membrane interface chamber 12′ can be drawn through the micro-conduits 40′ into the testing chamber 54′ so that a reaction can be performed to determine the presence of molecules. Such reactions can be ELISA assays, or chromatography as described above, a PCR assay, an absorbance assay, a calorimetric assay, a solid-phase immunoassay, an enzyme immunoassay, a fluorescent immunoassay, or any other suitable reaction or assay.

The sensors/sensor array 14′ include at least one potentiometric and one amperometric sensor as described above. The sensor/sensor array 14′ can be covered by an array membrane as described above for the purpose of potentiometric transduction or to provide selected access by certain molecules to the sensor. The sensor/sensor array 14′ is manufactured and made of materials as described above.

The testing chamber 54′ further includes an evaporative waste disposal chamber 66′ as shown in FIGS. 35 and 36. The evaporative waste disposal chamber 66′ allows fluids from the testing chamber 54′ to be removed from the agent delivery device 10′ through evaporation once a reaction has been performed. The evaporative waste disposal chamber 66′ can be operatively connected to the testing chamber 54′ by micro-conduits 40′, and can be manufactured in the same manner and with materials described above for the chambers 12.

The testing chamber 54′ can further include a signal transmitter 68′ for sending a signal either by telemetry to a microprocessor and/or to a second device for dispensing a molecule, or by electronic connection to another site on the agent delivery device 10′. The signal transmitter 68′ can be any suitable signal transmitter 68′ and can be operative integrated in the agent delivery device 10′ at any suitable location. The signal can be used to report the results of the reaction(s) in the testing chamber 54′ and can be displayed to a user, either on the agent delivery device 10′ itself or on a separate microprocessing device. The signal can have a unique encoding so as to distinguish from other signals coming from other devices. The signal transmitter 68′ can operate in any suitable band such as but not limited to the wireless medical telemetry services (WMTS) band, radio frequency, or other similar frequencies capable of operating the device of the present invention. Any suitable signal transmitter 68′ can be used. For example, Bluetooth™ technology can be utilized. The telemetric signal can come from a remote device such as from a handheld control, or from a main station such as a nurse's station or any other base for monitoring people.

The agent delivery device 10′ can operate in an active or in a passive manner. During active operation, a user can operate a control 70′ on the agent delivery device 10′ to acquire a sample of interstitial fluid from the membrane 60′ and perform a reaction on the captured interstitial fluid in the testing chamber 54′, and the user can monitor the results. During passive operation, the agent delivery device 10′ can automatically acquire a sample of interstitial fluid at a predetermined programmable time interval and perform a reaction in the testing chamber 54′ for a continuous monitoring of a user's interstitial fluid.

The agent delivery device 10′ can further include at least one reservoir 72′ for storing reservoir fluid being operatively connected to the membrane interface chamber 12′ and/or testing chamber 54′ by micro-conduits 40′, as shown in FIG. 38. The reservoir fluid can be any desired fluid in cleaning/calibrating the membrane interface chamber 12′ and the testing chamber 54′ such as buffer solution, calibration solution, and wash solution.

The body portion 13′ can be integrated with a patch 74′ including an adhesive backing for removable attachment to the membrane 60′, shown in FIGS. 35 and 36. The patch 74′ can optionally cover the entire body portion 13′. Adhesive can also be applied to the bottom edges 76′ of the body portion 13′ without a patch 74′ for application to the membrane 60′. Skin permeation enhancers can be applied to the adhesive such as liposomes, menthol derivatives, or glycerol derivatives to enhance the permeation of molecules through the membrane 60′.

For example, CPEs are compounds that enhance the permeation of drugs across the skin. These CPEs increase skin permeability by reversibly altering the physicochemical nature of the stratum corneum, the outer most layer of skin, to reduce its diffusional resistance. These compounds increase skin permeability also by increasing the partition coefficient of the drug through skin and by increasing the thermodynamic activity of the drug in the vehicle. Chemicals such as liposomes, menthol derivatives or glycerol derivatives cam enhance the permeation of drugs through the skin.

Based on the chemical structure of penetration enhancers (such as chain length, polarity, level of unsaturation and presence of some special functional groups such as ketones), the interaction between the stratum corneum and penetration enhancers may vary which results in significant differences in penetration enhancement. Two very potent enhancers that can be considered are decylmetyl sulfoxide (DMSO) and oleic acid that act by altering the level of hydration or degrading proteins and membrane lipids. Also, oleic acid incorporates into the skin lipids and disrupts molecular packing of the membrane, alters the level of hydration, and allows faster drug penetration.

Other CPEs that can be used for the enhancement of Transdermal delivery (TDD) extraction of the glucose are as follows. It has been found that polyunsaturated fatty acids PUFA-Linoleic (LA), alpha-linolenic (ALA), and arachidonic acids enhance skin permeation to a greater extent than monounsaturated fatty acids. The enhancement effects of fatty acids on penetration through the stratum corneum are structure-dependent, associated with the existence of a balance between the permeability of pure fatty acids across stratum corneum and the interaction of the acids to skin lipids. Cod-liver-oil can also be used. The enhancing effect of the marine products could generally be associated with their content of free unsaturated fatty acids. As potential skin penetration enhancers, studies have demonstrated that the permeation enhancing effect of I-menthol is significant with short lag time. The promoting activity of the ethyl ether derivative of Menthol is the greatest of all menthol derivatives. Studies have shown that this derivative is the most promising compound with the greatest action and relatively low skin irritancy. Studies have elucidated the mechanism of skin permeation enhancement and it was concluded that the increase in skin flux, up to eight times the base line, could be attributed to the effect of menthol on the skin barrier properties. Squalene was found to be a very effective skin permeation enhancer. 12% of the human sebum is composed of Squalene to which is attributed the natural moisturizing effect of the sebum. Studies also showed the skin soothing effect of Squalene. Studies concluded that glycerol monoethers derived from linear saturated fatty alcohols are very effective permeation enhancers. While specific embodiments are disclosed herein, they are not exhaustive and can include other suitable designs and systems that vary in designs, methodologies, and transduction systems (i.e., assays) known to those of skill in the art. In other words, the examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

The agent delivery device 10′ of the present invention can be used to monitor many different molecules in interstitial fluid. For example the interstitial fluid can be monitored for low molecular weight proteins to detect cancer, metabolic disease, heart function, or liver function. The low-molecular weight proteomic analysis of serum, which is believed to contain multitudes of biological markers that could provide the means for assessing an individual's health, is difficult to analyze due to the need to perform extensive fractionation to remove large proteins prior to mass spectrometric analyses. In addition, obtaining serum is necessarily an invasive procedure. Interstitial Fluid (ISF), the extracellular fluid surrounding cells, is a microcosm of human serum containing proteins and peptides at approximately thirty percent of the concentration found in serum. This was determined by applying a standardized suction technique to sample plasma proteins in dermal interstitial fluid serially for 5 to 6 days from a suction-induced skin mini-erosion. Since ISF can be obtained non-invasively, through the skin using various established techniques, and since the composition of ISF is closely related to that of serum plasma, it is an ideal body fluid to sample and monitor for biological markers.

The one “limitation” of non-invasive interstitial fluid sampling, the difficulty with which large molecules pass through the stratum corneum (SC) layer of the skin, serves as an advantage when attempting to sample and characterize the LMW components of the ISF proteome. In this respect, the stratum corneum is a natural filter allowing only the smaller LMW components to pass through while retaining the larger molecular weight components, thus eliminating the need to perform extensive fractionation of the sample. Whereas fractionation of serum to remove the high molecular weight proteins requires hours or days to perform, the agent delivery device 10′ has the potential to obtain ISF samples, containing only low molecular weight proteins, within minutes. Such an agent delivery device 10′, with the incorporation of specific marker sensors and readout circuitry, allows an individual's health status to be assessed immediately.

In a further embodiment, the micro-device 10 is a agent delivery device 10″ including a body portion 13″ housing a membrane interface chamber 12″ and a molecular delivery apparatus 82″ for delivering molecules through the membrane 60″. The agent delivery device 10″ is small, on the order of a few square centimeters or less. The agent delivery device 10″ is manufactured and made of materials essentially as described above for the agent delivery device 10, and integrates the circuitry, microfluidic devices 48, and other elements of the agent delivery device 10 as described above.

The molecular delivery apparatus 82″ can be at least one reservoir 72″ operatively attached to the membrane interface chamber 12″ by micro-conduits 40″. The reservoir(s) 72″ can be controlled by microfluidic valves 50″ and microfluidic pumps 47″, as described above. Agents are stored in the reservoir 72″ until the need for administration when they are released into the membrane interface chamber 12″ to be administered through the membrane 60″. Other fluids can also be stored in the reservoir 72″, such as wash fluid described above or any other suitable fluid. Additionally, the device 10 of the present invention can include numerous reservoirs 72″. The reservoirs 72″ do not have to all contain the same agent. Instead, adjacent reservoirs 72″ can contain agents that work in concert with one another. For example, one reservoir 72″ can contain the needed agent and the next reservoir 72″ can contain a skin healing agent or chemical enhancer that aids in the delivery of the needed agent. The benefit of such a configuration is a limit in potential skin irritation at the site of agent administration. Alternatively, the reservoir 72″ can be layered with different agents being encapsulated in the layers.

An electrode(s) 22″ can also be operatively attached to the membrane interface chamber 12″ for electrophoresic/iontophoretic delivery. Alternatively, other devices can be affixed to the membrane interface chamber 12″ to cause the agents to be released from the reservoir 72″. Preferably, the device is something that can administer electrons to the reservoir 72″ in order to release the agent from the reservoir 72″.

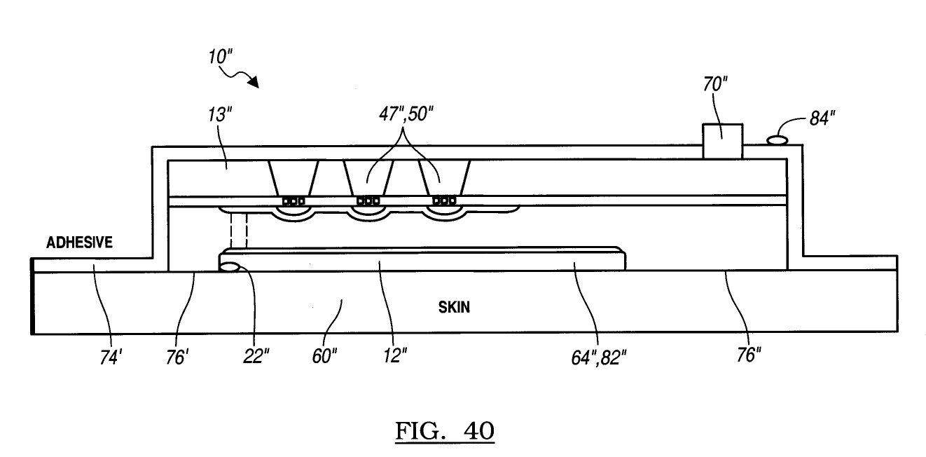

As shown in FIG. 40, the molecular delivery apparatus 82″ can also be an electrolyte polymer membrane 64″ with electrodes 22″ operatively attached, fitting inside the membrane interface chamber 12″, as described above. Embedded in the electrolyte polymer membrane 64″ are molecules which can be released by an electric current produced by the electrodes 22″, causing the molecules to be administered through the membrane 60″.

During active operation, a user can operate a control 70″ on the agent delivery device 10″ to deliver molecules from the reservoir 72″. The control 70″, when activated, causes the microfluidic pumps 47″ and microfluidic valves 50″ to release molecules from the reservoir 72″, or the control 70″ causes the activation of electrodes to release molecules from the electrolyte polymer membrane 64″.

The molecular delivery apparatus 82″ can also include signal receiver 84″ to receive a telemetric signal. The signal receiver 84″ can be any suitable signal receiver 84″ and can also be operatively integrated in the device 10″ in any suitable location. The telemetric signal can activate the microfluidic pumps 47″ and the microfluidic valves 50″ to release molecules in the reservoir 72″ into the membrane interface chamber 12″ to be delivered to the membrane 60″. The telemetric signal can also activate the electrodes 22″ to stimulate the release of the molecules in the electrolyte polymer membrane 64″ to be delivered to the membrane 60″. The telemetric signal can be any signal as described above. The telemetric signal can come from a remote device such as from a handheld control, or from a main station such as a nurse's station or any other base for monitoring people.

The body portion 13″ can be integrated with a patch 74″ including an adhesive backing for removable attachment to the membrane 60″. The patch 74″ can optionally cover the entire body portion 13″. Adhesive can also be applied to the bottom edges 76″ of the body portion 13″ without a patch 74″ for application to the membrane 60″. Skin permeation enhancers, as disclosed above, can be applied to the adhesive such as liposomes, menthol derivatives, glycerol derivatives, linoleic acid, or menthone to enhance the permeation of molecules through the membrane 60″.

The agent delivery device 10″, with any of the structure described above and in active or passive delivery operation as described above, can be used to deliver molecules such as, but not limited to, nicotine for cessation of smoking, an anti-malarial agent, an antibiotic, and a gonadotropin releasing hormone for positive or negative control of fertility as further described in the examples below.

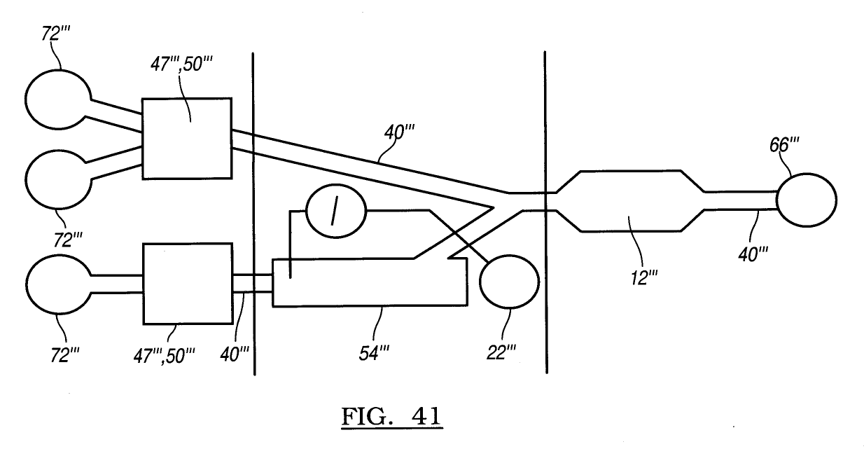

The agent delivery system 10′″ includes a transmembrane fluid capturing chamber 12′″, also called a membrane interface chamber 12′″, with electrodes 22′″ operatively integrated for capturing interstitial fluid through a membrane 60′″, a testing chamber 54′″ for detecting molecules in captured interstitial fluid, and a molecular delivery apparatus 82′″ for delivering molecules through the membrane 60′″, all essentially as described above. The agent delivery system 10′″ is small, on the order of a few square centimeters or less. The agent delivery system 10′″ is made from essentially the same materials and manufactured in the same method as described for the agent delivery 10 above. The agent delivery system 10′″ is shown in FIGS. 41, 42, and 43.

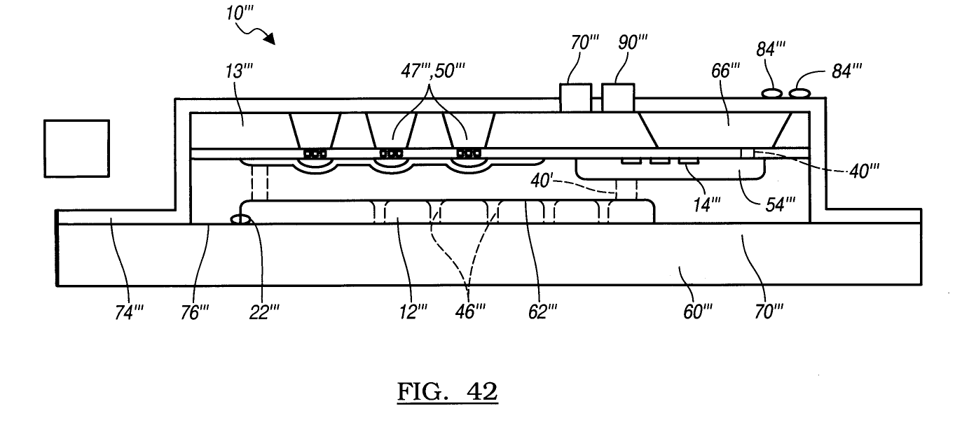

The agent delivery system 10′″ can include one body portion 13′″ having the membrane interface chamber 12′″, the testing chamber 54′″, and the molecular delivery apparatus 82′″ as shown in FIGS. 41 and 42. In this configuration, the membrane interface chamber 12′″ serves as both the site for the acquisition of interstitial fluid from the membrane 60′″ and the site for delivery of molecules into the membrane 60′″. The membrane interface chamber 12′″ can include supports 46′″ or an electrolyte polymer membrane 64′″ as described above.

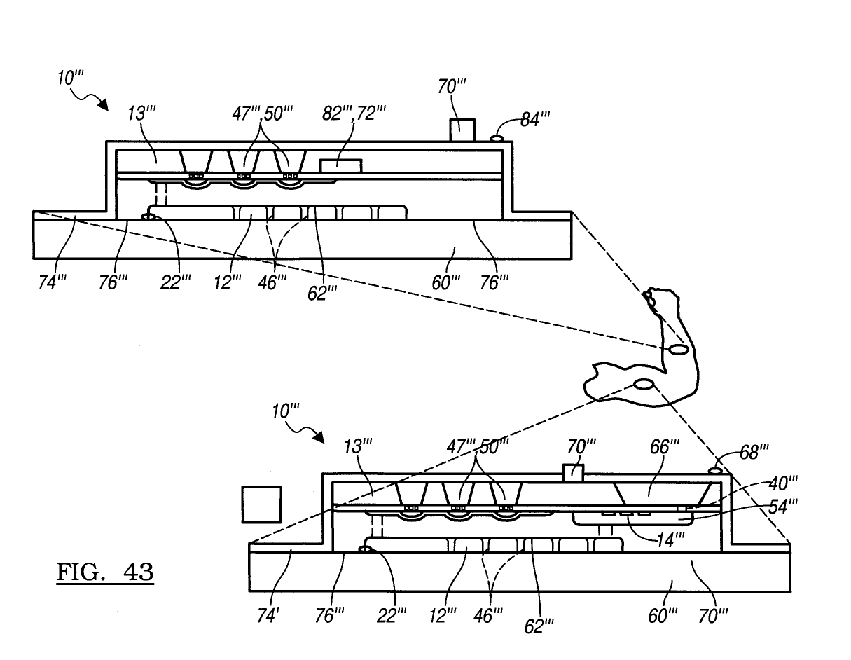

Alternatively, the agent delivery system 10′″ can include a body portion 13′″ having the membrane interface chamber 12′″ and the testing chamber 54′″ (essentially the agent delivery device 10′), and a second body portion having the molecular delivery apparatus 82′″ and a second membrane interface chamber (essentially the agent delivery device 10″), as shown in FIG. 43. The second membrane interface chamber has the same characteristics as the membrane interface chamber 12″ in the agent delivery device 10″ described above. In this configuration, the interstitial fluid acquisition and the delivery of molecules can occur at different places on a user's body. The membrane interface chamber 12′″ and the second membrane interface chamber can both include either supports 46′″ or an electrolyte polymer membrane 64′″, or a combination (one body portion 13′″ or 86″ has supports 46′″ and the other has an electrolyte polymer membrane 64′″). The body portion 13′″ can be placed on a membrane 60′″ at one location on the body, and the second body portion can be placed on another membrane 60′″ at another location on the body. The body portions 13′″ and 86″ can also be positioned so that one is in vivo while the other is ex vivo.

The body portion 13′″ and second body portion can be integrated with a patch 74′″ including an adhesive backing for removable attachment to the membrane 60′″. The patch 74′″ can optionally cover the entire body portions 13′″ and 86″. Adhesive can also be applied to the bottom edges 76′″ of the body portions 13′″ and 86′″ without a patch 74′″ for application to the membrane 60′″. Skin permeation enhancers can be applied to the adhesive such as liposomes, menthol derivatives, or glycerol derivatives to enhance the permeation of molecules through the membrane 60′″.

The agent delivery system 10′″ can further include at least one reservoir 72′″ for storing reservoir fluid being operatively connected to the membrane interface chamber 12′″ and/or testing chamber 54′″, and the second membrane interface chamber 88′″ by micro-conduits 40′″, as described above. The reservoir fluid can be any desired fluid in cleaning/calibrating the membrane interface chamber 12′″ and the testing chamber 54′ such as buffer solution, calibration solution, and wash solution. The reservoir 72′″ can also store molecules to be delivered. On the second body, at least one reservoir 72′″ stores molecules when the second membrane interface chamber includes supports 46′″.

Acquisition of interstitial fluid and delivery of molecules through the membrane 60′″ can be accomplished in an active or a passive manner. During active operation, a user can operate a control 70′″ on the body portion 13′″ to acquire a sample of interstitial fluid from the membrane 60′″ and perform a reaction on the captured interstitial fluid in the testing chamber 54′″, and the user can monitor the results. Based on the results, the user can then operate a second control 90′″ on the body portion 13′″ or on the second body portion to deliver molecules from either a reservoir 72′″ or from an electrolyte polymer membrane 64′″, as described above.

During passive operation, the agent delivery device 10′″ can automatically acquire a sample of interstitial fluid at a predetermined programmable time interval and perform a reaction in the testing chamber 54′″ for a continuous monitoring of a user's interstitial fluid. The results of the reaction can be sent from the testing chamber 54′″ to the molecular delivery apparatus 82′″ to actuate the release of molecules from either the reservoir 72′″ or from the electrolyte polymer membrane 64′″. In this manner, the agent delivery device 10′″ operates in a continuous monitoring and delivering method. The passive mode of operation is useful in the monitoring and delivery of therapeutics with narrow therapeutic windows.

Telemetry can be used in both the active and passive methods of operation. The testing chamber 54′″ can include a signal transmitter 68′″ as described above. The molecular delivery device 82′″ also includes a signal receiver 84′″ as described above. The signal transmitter 68′″ and the signal receiver 84′″ operate essentially as described above, acquiring a sample and transmitting a signal with data to a receiver, and receiving a signal with data to activate delivery of molecules, and optionally transmitting/receiving signals to/from a main station.

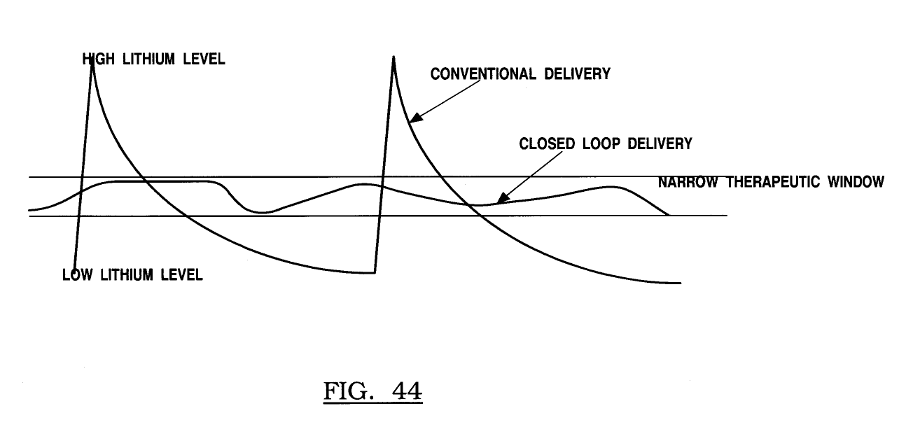

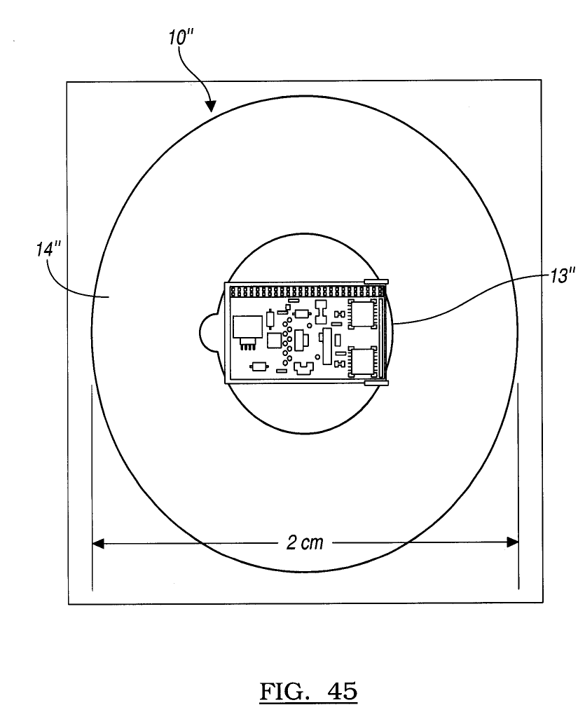

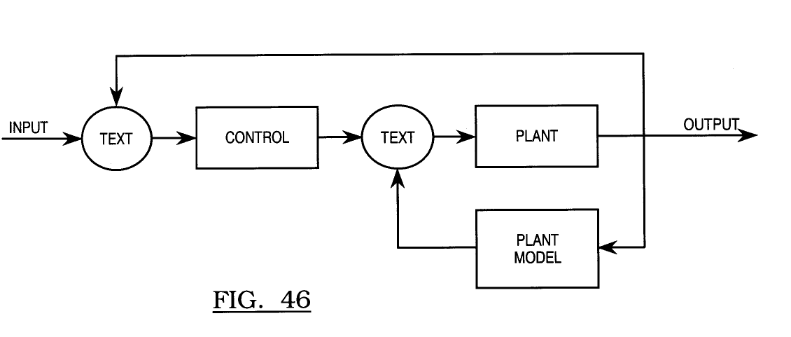

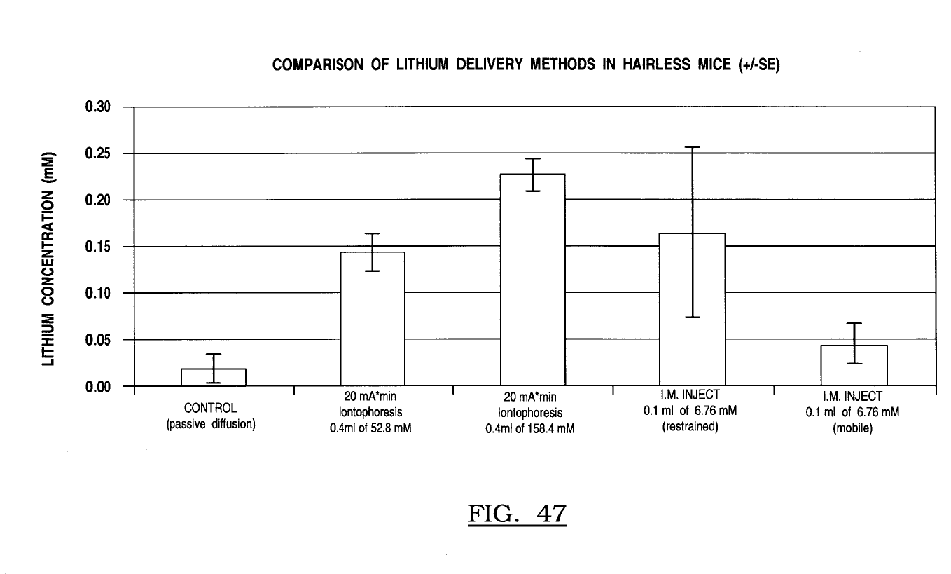

The telemetry in the agent delivery device 10′″ can also operate in an additional method of a closed loop system for real-time monitoring. The closed loop system causes interstitial fluid to be obtained periodically from the membrane interface chamber 12′″. Then, the captured interstitial fluid is tested in the testing chamber 54′″. A signal is generated based on the data from the testing chamber 54′″. This signal of feedback from the testing chamber 54′″ is sent from the signal transmitter 68′″ to the signal receiver 84′″, where it is interpreted and thereby actuating the release of molecules by the molecular delivery apparatus 82′″ for administration through the membrane 60′″. The closed loop system can operate with one body portion 13′″ and also with the second body portion. When the second body portion is included, the signal from the signal transmitter 68′″ on the body portion 13′″ travels to the signal receiver 84′″ on the second body portion 86″. Using a closed loop system provides higher control in dosing and response as shown in FIG. 44, especially with drugs having a narrow therapeutic window (such as lithium), and is advantageous over other methods of drug delivery.

The agent delivery device 10′″ can automatically dispense molecules at a predetermined programmable time interval in a pulsatile release manner. In other words, molecules can be automatically released in pulses from the reservoir 72′″ or the electrolyte polymer membrane 64′″ can be automatically stimulated by the electrodes to release molecules in pulses. Pulsatile delivery can be used with telemetry and a closed loop system. For example, the membrane interface chamber 12′″ can acquire interstitial fluid, test it in the testing chamber 54′″, the signal transmitter 68′″ can send a signal to the signal receiver 84′″, which actuates the release of molecules by the molecular delivery apparatus in a pulsatile manner.

For some types of drugs, it is preferred to release the drug in “pulses,” wherein a single dosage form provides for an initial dose of drug followed by a release-free interval, after which a second dose of drug is released, followed by one or more additional release-free intervals and drug release “pulses.” Pulsatile drug delivery is useful, for example, with active agents that have short half-lives and must be administered two or three times daily, with active agents that are extensively metabolized presystemically, and with active agents which lose the desired therapeutic effect when constant blood levels are maintained. These types of agents have pharmacokinetic-pharmacodynamic relationships that are best described by a clockwise “hysteresis loop.” A drug dosage form that provides a pulsatile drug release profile is also useful for minimizing the abuse potential of certain types of drugs, i.e., drugs for which tolerance, addiction and deliberate overdose can be problematic and creates a more natural drug delivery. Further, pulsatile delivery is advantageous for drugs that have a narrow therapeutic window, usually requiring close monitoring and a smaller dose at a more frequent interval. The amount of drug in the body can be controlled easier with pulsatile delivery, maintaining effectiveness while reducing side effects. Several drugs having a narrow therapeutic window include, but are not limited to, levothyroxine, phenytoin, warfarin, theophylline, lithium, digoxin, and 5-fluorouracil.

Pharmaceutical companies employ a variety of approaches for overcoming the problem of pre-systemic elimination in oral drug administration. Included among these approaches is the use of physical and chemical agents to delay drug metabolism, alternate delivery routes to bypass hepatic metabolism and pulsatile delivery systems, mainly in the form of layered pills or capsules for oral intake, to control the rate of drug release. Despite the efforts necessary to develop these techniques, they have failed to address the problems associated with the continuous and/or oral administration of drugs. The agent delivery device 10″ can overcome previous techniques by providing more accurate pulses of molecules. With a closed loop system, the agent delivery device 10′″ can also closely monitor molecule levels in the body and give pulses of required molecules more accurately when needed.

The agent delivery device 10′″ can be used for many different applications such as, but not limited to, analyzing captured interstitial fluid for melatonin and delivering molecules including melatonin for treating a sleeping disorder, analyzing captured interstitial fluid for glucose and delivering molecules including insulin for treating diabetes or stress, analyzing captured interstitial fluid for lithium and delivering molecules including lithium for treating a psychological disorder, delivering molecules including butylcholinesterase or atropine for acute treatment of chemical warfare agents, or delivering hormones, buserelin, methylphenidate, or mecamylamine. Several of these applications are further described in the examples below.

For example, glucose concentration in blood can be used to determine metabolic status as well as to assess the degree of psychological and physical stress experienced by the individual, by providing indications of their homeostatic condition and providing evidence of stress.

In addition to lithium and other psychotic drugs, such as valproate and haloperidol, the device can non-invasively monitor, in real-time, hundreds of other biological markers such as blood electrolytes, blood ions, glucose, biologically active substances, pharmacological drugs, drugs of abuse, pesticides, hormones, etc. Further, it is possible to customize the system to automatically deliver different types of medication in precise amounts. For example, one application allows insulin-dependent diabetics to closely regulate their blood sugar and maintain a healthy state of euglycemia. With a focus on controlled lithium delivery and the potential for many other applications, the LDMS revolutionizes how diseases are treated today and make proper regulation an attainable goal for everyone.

The device 10 of the present invention can also be used for the treatment of diabetes, manic depression, anxiety disorders, smoking cessation, antibiotic application, or hormonal therapy for fertility, infertility, growth disorders, sleep disorders, etc. or application in the cosmetic industry to remove facial skin wrinkles, acne scars, and other cosmetic treatment to facial features and to return plasticity to aging or full thickness burn damaged skin. The system of the present invention can be utilized to target and induce the formation of collagen, in the appropriate orientation and at a high rate of deposition, in a non-invasive manner. As a result, the skin's elasticity and plasticity can be improved and/or restored.

In treating the skin, the agent delivery device 10 of the present invention is capable of laying a scaffold of precursor substrates in an individual. The scaffold can be established in the epidermis, dermis, subcutaneous fat, or in any other layer within the body of an individual. The scaffold is defined as a supporting framework of precursor substrates wherein the precursor substrates are aligned and/or oriented in a manner that aids in the formation of collagen. Alignment and/or orientation of precursor substrates occur via electromagnetic stimulation. The electromagnetic stimulation increases the growth rate and control of orientation of the newly formed collagen molecules.

While specific embodiments are disclosed herein, they are not exhaustive and can include other suitable designs and systems that vary in designs, methodologies, and transduction systems (i.e., assays) known to those of skill in the art. In other words, the examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### PREFERRED EMBODIMENTS

### Examples

### Category 1

The treatment of diseases and physiologic conditions with the agent delivery system provides a method for delivering the treatment agent directly via the skin to avoid the challenges presented by oral delivery and use of bolus injections. The agent delivery system may be used as an agent delivery system alone or in conjunction with a feedback controller unit. The agent delivery system may be programmed to deliver an agent via pulsatile administration. The interval and dose may be based upon receptor turnover, regeneration or reactivation rates. The interval may also be used in antibiotic therapy to correspond to parasite lifespan or life-cycle.

The examples provided for this agent delivery system category involves: a pulsatile delivery device, an automated controller, agent:polymer matrix, a biocompatible membrane and adhesive to attach the delivery reservoir to the skin, pulsed timing programmed to reflect receptor turnover, feedback control data or a ramp down application. The unit has an integrated USB port and may be adapted for wireless signal transmission.

The present invention for this category may be utilized, but not limited to administering: an anti-malarial agent, an antibiotic, nicotine for cessation of smoking, or a gonadotropin releasing hormone for positive or negative control of fertility. These examples are for illustrative purposes and intended to be descriptive rather than limitations.

### Example 1

### Malaria

A wearable anti-malarial pulsatile administration device (AMPAD) that delivers anti-malarial drugs in a transdermal, pulsatile manner was developed. The AMPAD includes a micro-iontophoresis system, constructed using MEMS and CMOS technologies, and a polymer matrix electrolyte reservoir that contains the drug. The system delivers precise square wave pulses of antibiotic through the skin to increase the efficacy of treatment, as well as compliance to anti-malarial prophylaxis, by eliminating the side effects that result from oral administration.

Polymer matrix electrolytes have been shown to be ideal for storage and delivery of molecules, such as lithium and lidocaine, since the polymers trap the molecules and release them only when a current is applied to the matrix. The microcircuitry, manufactured using CMOS technology, is integrated into a single silicon chip. The device is powered by a thin film battery, built into the protective casing that surrounds the unit, providing a self-contained device the size of a band-aid. The protective casing as well as the entrapment of the molecule in a solid matrix, which is released only when current is applied, provides a fail-safe mechanism such that in the event of damage to the device, the patient can be protected from inadvertent exposure to the drug. Such a device is needed to increase compliance, reduce the costs, and increase the efficacy of antibiotic therapy.

None of the prior art methods of transdermal delivery are very efficient (requiring large patches for an effective dose) or are capable of delivering an anti-malarial agent/antibiotic in a pulsatile manner, as the agent delivery system described above. To aid in the delivery of hydrophobic antibiotic molecules, the fluid delivery device uses an electrolyte polymer membrane, to trap the molecule and release it when current is applied. The agent delivery system is a wearable transdermal patch that incorporates a micro-iontophoresis system, constructed using MEMS and CMOS technologies, and an electrolyte polymer membrane containing sufficient drug to deliver precise square wave pulses of antibiotic to increase the efficacy of treatment, as well as compliance to anti-malarial prophylaxis, by eliminating the side effects that result from oral administration.

There are a number of anti-malarial drugs currently in use. For the best protection against malaria, mefloquine, doxycycline, chloroquine, atovaquone/proguanil, or primaquine are commonly prescribed. However, the number of effective drugs available to treat malaria is small and the rate at which resistance is growing is outpacing the development of new antimalarials. The main obstacle to malaria control is the emergence of drug-resistant strains of the parasite P. falciparum, the deadliest of all the malaria pathogens.

The lipophilicity of anti-malarial drugs makes them good candidates for transdermal absorption. Moreover, the use of a pulsatile transdermal anti-malarial drug delivery system provides a means to decrease or eliminate the development of resistance to these drugs. The technology combats both the problem of resistance and the problem of non-compliance to oral administration of antibiotics.

Researchers have been investigating the transdermal delivery of various anti-malarial drugs including the following. Triclosan is widely used as an anti-bacterial agent and it has recently been demonstrated that this compound has anti-malarial properties. Its high lipophilicity makes it a potential candidate for delivery across the skin. It was determined that a simple transdermal patch could deliver a therapeutic in vivo dose of primaquine across full-thickness excised human skin, with possibilities for the treatment and prophylaxis of Plasmodium vivax, P. ovale and P. falciparum forms of malaria. Researchers have accumulated data that suggests (1) significant amounts of doxycycline, a potent anti-malarial drug, can be administered into and across human skin; (2) Migliol 840 is a potentially useful enhancing vehicle; and (3) significant amounts of drug were delivered transdermally. In the first 3 hours following introduction of erythromycin lactobionate, 1.85 mg/cm² crossed human epidermis. Given that a dose of 50 mg may exert prokinetic effects in vivo in man, increasing the patch size to approximately 28 cm² should provide therapeutic levels of drug within 3 hours.

To aid in the delivery of hydrophobic antibiotic molecules, the present invention uses an electrolyte polymer matrix, to trap the molecule and release it when current is applied. Polymer electrolyte films have been shown to be useful for electrotransport of drugs, e.g., lidocaine hydrochloride and lithium chloride. The polymers are cast from solutions of poly(etheleneoxide) (PEO) and various drug salts using either water (for hydrophilic molecules) or an acetonitrile/ethanol mixture (for hydrophobic molecules) as the casting solvent. AC impedance analysis demonstrates that the conductivity of the films vary between 10⁻⁶ and 10⁻³ cm⁻¹, depending on the salt, casting solvent, and temperature.

In addition to antibiotic delivery, the device of the present invention can also be used for the delivery of other hydrophobic and hydrophilic drugs and hormones. The device's ability to deliver drugs in a pulsatile manner has proven to have advantages over continuous delivery. As previously indicated, the pulsatile delivery of drugs increases their effectiveness while simultaneously decreasing side-effects. The device's ability to deliver drugs in a transdermal manner has proven to have advantages over oral administration, including the need to address pre-systemic elimination. Pharmaceutical companies employ a variety of approaches for overcoming the problem of pre-systemic elimination in oral drug administration. Included among these approaches is the use of physical and chemical agents to delay drug metabolism, alternate delivery routes to bypass hepatic metabolism and pulsatile delivery systems, mainly in the form of layered pills or capsules for oral intake, to control the rate of drug release. Despite the efforts necessary to develop these techniques, they have failed to address the problems associated with the continuous and/or oral administration of drugs.

To meet this objective, an anti-malarial antibiotic was incorporated into a polymer electrolyte and the polymer was cast into a mold the size of a band-aid, approximately 2 cm in diameter. Polymer electrolytes are solid-like materials formed by dispersing a drug in a high molecular weight, lipophilic polymer. In essence, the molecule is trapped within the polymer until the application of an electric current. Application of electric current causes the porosity and diameter of the pores of the polymer to increase, hence providing controlled release of the drug. The technology allows molecular concentrations as high as 4 molar to be incorporated into the matrix.

The patch was applied to human skin samples using an in vitro iontophoresis apparatus to measure the flux of antibiotic that crosses the skin after application of electric current to demonstrate that enough transdermal antibiotic is delivered transdermally to mimic serum levels achieved by oral administration.

Films of PEO (RMM: 4,000,000, Aldrich) mixture were prepared using a standard solvent casting technique for the preparation of polymer electrolyte films. The compositions were in the form PEOn:antibiotic (where n=10 or 20). This represents the molar ratio of the ethylene oxide (EO) repeating unit to antibiotic. PEO10:antibiotic represents 1 molecule of antibiotic associated with 10 EO units. For each preparation, 1 g of PEO was used and the mass of antibiotic to be used was calculated by dividing the molecular mass of the antibiotic by the molar ratio of 10 and the molecular mass of EO repeat unit (i.e. 44).

The calculated mass of antibiotic was then added to 1 g of PEO in 50 mL of distilled water (for hydrophilic molecules) or acetonitrile:ethanol (for hydrophobic molecules) and stirred until complete dissolution. The mixture, which was a viscous solution, was then cast into polystyrene 2 cm diameter culture dishes. Before the polymer had cured, a loop of platinum wire was inserted into the solution such that it was firmly held in place by the cured polymer. The solution was then covered and the solvent was allowed to evaporate at room temperature. The film was then peeled from the well and stored in a sealed plastic bag over silica gel in a desiccator.

A pressure sensitive adhesive (PSA), such as an acrylic emulsion, was applied to the bottom of the patch to provide a tight seal between the polymer and skin. New polymer adhesives have become available to advance transdermal technology. The polymers have been modified to improve solubility and drug diffusion with little change in adhesive and cohesive properties. 3M's Latitude™, and CORPLEX™, both of which are polymer adhesives, has a versatile range of properties for water sorption and adhesion to moist skin. Long-term applications may require a more durable adhesive similar to Mastisol™, a surgical adhesive containing gum mastic. It is best removed using the product Detachol™, which contains petroleum distillates.

The delivery electrode was incorporated into the polymer-antimalarial matrix, which was placed on top of the skin in the donor compartment of the device, while the return electrode was inserted into the receptor compartment.

Construct electrolyte polymer delivery pad: To measure electrochemical degradation that was caused by iontophoresis, thin layer chromatography cab be used. An initial experiment was performed to determine the sensitivity of this method and the migration pattern of primaquine. Silica gel plates were used to spot 50, 5, 0.5, and 0.05 μg of primaquine. n-butanol:acetic acid:water (5:3:2) was used as the solvent and the chromatography was run for four hours at room temperature.

It is apparent from this experiment that the system permits any degradation products, due to electrochemical degradation, to be visualized easily since all degradation products are of lower molecular weight and would appear as spots below the primaquine spots pictured above. However, a better developing reagent is needed, such as Dragendorff's reagent since the iodine vapor also colors the TLC silica gel and reduces the resolution contrast considerably.

To quantify the amount of primaquine that can be delivered transdermally, the absorbance of UV light by primaquine was investigated. Using a BioTek Synergy HT plate reader, a full absorbance spectrum was run using two different primaquine concentrations.

The absorbance spectrum shows an absorbance peak at 340 nm wavelength. The wavelength was used to measure the dose response of primaquine. A standard curve was prepared using concentrations of 0.03125-0.5 mg/ml, in duplicate. The absorbance at 340 nm was plotted vs. primaquine concentration.

Researches have found that 10 mg of primaquine can be delivered, transdermally, within 24 hours to achieve therapeutic plasma concentrations. Approximately 5% of the 10 mg dose is delivered passively each hour. The use of iontophoresis increases the delivery rate and transdermal flux.

Since the receptor compartment of the in vitro transdermal diffusion device is 5.0 ml, and assuming the maximum amount of primaquine delivered is 10 mg, the maximum concentration in the receptor compartment is 2.0 mg/ml. Estimating that 10 percent of the total amount of primaquine is delivered per pulse of current, direct measurement of the receptor compartment absorbance at 340 nm gives reliable primaquine concentrations using the same standard curve.

Casting of electrolyte polymer-primaquine matrix: A drug patch was prepared and tested for the ability to release the drug when current is applied.

The patch was prepared by casting PEO (polyethylene oxide, the electrolyte polymer) into a polydimethylsiloxane (PDMS) polymer mold and allowing it to dry at room temperature. The mold was prepared by casting 200 ml of a two part PDMS (Sylgard 184) mixture into a Petri dish containing a Teflon wafer at the bottom and surrounded by a foil sleeve. After curing at 90° C. for 30 minutes, a 1 cm cork borer was used to bore a hole into the PDMS and create a mold. This type of mold is needed since the polymer-drug mixture sticks to most surfaces. The Teflon-PDMS mold allows the patch to be released from the mold easily.

A mixture of polyethylene oxide (PEO) and primaquine was made by first dissolving 0.1 g of PEO in 10 ml of distilled water. The mixture was heated to 100° C. until dissolved. After cooling, 0.102 g of primaquine was added and shaken on a Vortex mixer until dissolved. 2.5 ml of the PEO-primaquine mixture was added to the mold and the solution was allowed to dry at room temperature. A platinum electrode wire loop was inserted into the mold along with the PEO-drug mixture.

Periodically, over the course of a week, the solution was topped off with more of the PEO-primaquine mixture until a total of 8.0 ml was added and dried. The result was a PEO-primaquine patch containing 80 mg of drug. After drying, the patch was coated with a silicone pressure sensitive adhesive (BIO-PSA 7-4602), a hydrophobic adhesive that can be used to attach the patch to the skin, to determine the device's permeability to the drug.

To accelerate the drying time, it was thought that a better system would be one that provided a large surface area during drying. In this manner, the patches could be cut using the cork borer after the polymer-drug matrix had thoroughly dried. To do this, a second mold was created by coating a thin layer of PDMS onto the bottom of a 100 mm Petri dish and adding 100 ml of the PEO-drug mixture the plate, filled to the brim.

Prepare iontophoresis systems: To test the functionality of the electrolyte polymer to release primaquine when current is applied, the patch was suspended on the surface of a balanced salt solution while current was applied using the Phoresor II iontophoresis system. A 300 ohm resistor (to mimic the resistance of human skin) was soldered to a section of platinum wire and placed into the salt solution. The positive electrode was connected to the patch electrode and the negative electrode was connected to the resistor. Since primaquine is a positively charged molecule, migration is toward the negative electrode. A current dose of 80 mA*min was applied to the patch and 100 μl aliquots were sampled every 10 min.

After the electrodes were connected to the patch and placed in the reservoir, 100 μl samples were collected every 10 minutes. No current was applied for the first 20 minutes to determine if there was passive release of the drug. At 20 minutes, an 80 mA*min current dosage was applied to the patch and samples were collected. A “halo” of drug was apparent in the receptor compartment after 10 minutes of iontophoresis. Before sampling the receptor compartment, the contents were thoroughly mixed by aspirating the liquid several times with a pipette.

The 100 μl samples were placed in the well of a microtiter plate (2 samples per time point) and read at a wavelength of 340 nm. Since only a balanced salt solution was used in the receptor compartment, the only ultraviolet absorbing compound present is primaquine. The results indicate that only a minimal amount of Primiquine was released prior to application of current. After the onset of iontophoresis, the absorbance increased four fold.

The data from these experiments indicate the following: 1. The formulation used for the PEO-primaquine patch is suitable for fabricating the transdermal patch; 2. The pressure sensitive adhesive used is permeable to the drug and allows the flow of current; 3. There is minimal passive diffusion of primaquine from the patch with no current applied; 4. There is significant delivery of drug after the current is applied.

Casting of electrolyte polymer-primaquine matrix: To accelerate the drying time of the casting process, a mold was created by coating a thin layer of PDMS onto the bottom of a 100 mm Petri dish and adding 100 ml of the PEO-drug mixture the plate, until the plate is filled to the brim. This mixture was placed in the dark to dry for 1 week before cutting individual patches with a 1 cm cork borer.

While the polymer was still moist, platinum wire loops were placed in the polymer to dry. The loops can also be inserted after drying by placing 100 μl of dH₂0 over the area to solubilize the surface of the polymer/drug. After drying, the loop is firmly attached to the patch. A 1 cm cork borer was used to cut out individual patches for testing.

Since 1 gram of primaquine was added to the 100 mm plate (radius=5 cm, area=78.5 cm2), the amount of drug in the plate after casting and evaporation was 1 gram/78.54 cm2. Given that the patches were cut using a 1 cm cork borer (radius=0.5 cm, area=0.785 cm2), the concentration of drug in the patches was 1/100^{th} the total amount of primaquine in the mold or 10 mg of primaquine per patch.

Perform experiments to determine pulse delivery efficiency of antimalarial: Three types of skin membranes can be prepared for in-vitro transdermal delivery experiments: epidermal membranes with a thickness of approximately 0.1 mm, are prepared by heat, chemical, or enzymatic separation; split-thickness skin with a thickness of 0.2-0.5 mm are prepared using a dermatome; and full-thickness skin with a thickness of 0.5-1.0 mm. Since the main barrier to drug delivery for the skin is located in the stratum corneum, all three membrane types have been used for absorption studies. Moreover, since the capillary network begins just below the epidermis and is contained throughout the dermis, in-vitro flux determinations using full thickness skin may yield an over-estimate of the time required for the drug to reach the capillary network, since the time measured is the time needed to entirely bypass the capillary network and reach the receptor compartment of the diffusion cell.

For the initial transdermal studies and since most of the barrier function is contained in the stratum corneum, epidermal membranes (containing the stratum corneum and epidermal layers) were used for these experiments.

Human skin was obtained from the National Disease Research Interchange (NDRI), procured from an abdominoplasty procedure. The subcutaneous fat was removed using blunt dissection with a scalpel. The skin sample was placed in distilled water at 60 C for 1 minute to loosen the epidermal layer. Using forceps, the epidermal layer was removed by teasing it away from the dermis.

To visualize the integrity of the membrane and assure that there were no visible holes or tears, the membrane was viewed microscopically after placement in the permeation device using an inverted phase contrast microscope. In this manner, each epidermal membrane was examined before proceeding with the experiment to ensure its integrity.

Using a 1 cm cork borer, membrane discs were cut and inserted into a Mattek permeation device. The primaquine patch, fabricated and coated with adhesive as described in previous reports, was applied to the membrane and the donor compartment was attached and secured. The assembly was placed into a 25 mm culture dish containing 5.0 ml of phosphate buffered saline (PBS) at pH 7.4.

To determine the amount of passive diffusion, no current was applied to the device for 1 hour, at which time the first 200 μl sample (in duplicate) was taken from the receptor compartment and placed into the wells of a 96 well microtiter plate. A current dose of 80 mA*min at a current level of 4 mA was then initiated and samples were collected at 10 and 20 minutes. Immediately after the first iontophoresis dose was completed, a second 80 mA*min current dose also at a current level of 4 mA was applied and at the end of this dose, samples were collected. After 20 minutes with no current applied, the final samples were taken to again determine passive diffusion. The experiment was repeated three times with three membrane samples and three separate patches.

After completion of the experiment, a standard curve was prepared and 200 μl samples were placed into the microtiter plate. The UV absorbance at 340 nm was measured using a BioTek Synergy HT plate reader. The concentration of UV absorbing primaquine in the receptor compartment was determined by extrapolation to the standard curve, corrected for volume at the time of sampling. Minimal passive diffusion was observed before and after iontophoresis.

Unlike passive delivery patches, that increase the flux of drug delivery as the patch size increases, electrotransport is a function of the current applied and is independent of the size of the patch. For this reason, a smaller patch is better for pulsatile iontophoretic delivery since the amount of drug delivered between pulses is minimized.

Perform experiments to determine maximum deliverable dosage of antimalarial and stability: To determine the stability of the primaquine molecule after exposure to iontophoresis, the receptor compartment from one of the delivery experiments was dried down under nitrogen and reconstituted with 100 μl of dH₂O. Primaquine standards were prepared at 50 μg/10 μl, 5 μg/10 μl, 0.5 μg/10 μl, and 0.05 μg/10 μl. 10 μl samples were added to a silica gel plate with UV indicator. The TLC was developed using n-butanol:acetic acid:water (5:3:2) as the solvent and the chromatography was run for four hours at room temperature. The photograph shows a broad band for the receptor compartment contents indicating that a) intact primaquine is present, and b) there is more than one species of molecule present.

To prepare the patches, the previous casting method was modified by using smaller PDMS coated Petri dishes (35 mm) and drying in an oven at 60 C for 5 hours to reduce the drying time. This method gave patches that appeared less oxidized and retained the bright orange color of the primaquine.

A modified casting method for preparing the primaquine patches has been developed. 35 mm Petri dishes coated with PDMS were prepared and cured. To the Petri dish was added 15 ml of primaquine-PEO containing 1 g of primaquine and 2 g of PEO in 100 ml of distilled water. Platinum wire coils were inserted into the patch after 4 hours of drying time. A 1 cm cork borer was used to cut the patches from the mold.

Since 15 ml of primaquine-PEO containing 1 g/100 ml of primaquine is added to the mold, 0.15 g of total drug is distributed across the area of the plate. For the 35 mm Petri dish (radius=1.75 cm, area=9.62 cm2) the distribution of drug is 0.15 g/9.62 cm2=15.6 mg/cm2. Therefore, with a patch size of 1 cm (radius=0.5 cm, area=0.785 cm2), 12.25 mg of primaquine is contained in each patch.

After cutting the patches from the mold, the platinum wire was fed through a holder fashioned from the end of a 1 cc syringe needle plunger with a hole drilled through its length.

To hold the patch and mouse in place during the animal studies, a small rodent restrainer has been modified with Plexiglas brackets that attach to the base of the restrainer.

For the studies, mice were exposed to various currents and current dosages to determine the maximum dosage to deliver primaquine without harm to the animal. After exposure, the animals were sacrificed by decapitation and trunk blood can be collected. This was performed at 15 minutes, 30 minutes, and 60 minutes after exposure to determine the delivery profile. Sham mice, receiving no iontophoresis treatment were used.

For the extraction of primaquine and its metabolite carboxyprimaquine from whole blood, the procedure of Ward et al. was followed with some modifications. Briefly, 2 ml of 25% ammonia solution (specific gravity 0.91) was added and vortex mixed for 2 minutes. The mixture was extracted with n-hexane-ethylacetate (3.5:0.5, v/v) and centrifuged at 1000 g for 10 minutes to separate the phases. The organic phase was separated and evaporated to dryness under nitrogen. The residue was reconstituted with 25 μl of n-hexane-ethylacetate (3.5:0.5, v/v). The samples were run using silica-gel thin layer chromatography to qualitatively determine the presence or absence of primaquine in the blood for the patch treated and untreated animals, respectively.

Results and Technical Feasibility: In summary, since the therapeutic dosage of Primaquine for the treatment of malaria is 0.03 μg/ml, and assuming approximately 5 liters of blood in an adult human, it is necessary to deliver 150 μg of the drug to reach the therapeutic level. Research of the literature reveals Primaquine half-life values ranging from 3 to 9 hours. Therefore, 75 μg is required to be delivered every 3 to 9 hours to maintain the therapeutic level of the drug. Since 160 μg can be delivered in 40 minutes using electrotransport, the proposed AMPAD device is a viable alternative for maintaining therapeutic levels of the drug, avoiding the oral administration route and associated side effects and increasing compliance to the treatment regimen in soldiers and others. In addition, the ability to deliver square wave pulses of the drug reduces the development of resistance.

### Example 2

### GnRH

Gonadotropin-Releasing Hormone (GnRH), also known as luteinizing hormone-releasing hormone (LH-RH), plays a central role in the biology of reproduction. Various analogs have been used for an increasing number of clinical indications. The GnRH decapeptide (pyro-Glu-His-Trp-Ser-Tyr-Gly-Leu-Arg-Pro-Gly-NH₂ or p-EHWSYGLRPG-NH₂) is produced in neurons of the medial basal hypothalamus from a larger precursor by enzymatic processing. The decapeptide is released in a pulsatile manner into the pituitary portal circulation system where GnRH interacts with high-affinity receptors (7-Transmembrane G-Protein Coupled Receptors) in the anterior pituitary gland located at the base of the brain. In the pituitary, GnRH triggers the release of two gonadotropic hormones (gonadotropins): luteinizing hormone (LH) and follicle-stimulating hormone (FSH). In testes and ovaries, LH stimulates the production of testosterone and estradiol, respectively. FSH stimulates follicle growth in women and sperm formation in men. When correctly functioning, the pulse-timed release and concentration levels of GnRH are critical for the maintenance of gonadal steroidogenesis and for normal functions of reproduction related to growth and sexual development.

GnRH can be incorporated into a polymer electrolyte matrix at concentrations high enough to deliver therapeutic doses transdermally using iontophoresis. To meet this objective, GnRH was incorporated into a polymer electrolyte and the polymer was cast into a mold the size of a band-aid, approximately 2 cm in diameter. Polymer electrolytes are solid-like materials formed by dispersing a drug in a high molecular weight, lipophilic polymer. In essence, the molecule is trapped within the polymer until the application of an electric current. Application of electric current causes the porosity and diameter of the pores of the polymer to increase, hence providing controlled release of the drug. This technology allows molecular concentrations as high as 4 molar to be incorporated into the matrix.

The patch can be applied to human skin samples using an in vitro iontophoresis apparatus to measure the flux of GnRH that crosses the skin after application of electric current. To mimic intravenous delivery of GnRH, a pulse of 5-15 μg of the molecule needs to be delivered every 90 minutes.

Preparation of polymer-GnRH films: Films of PEO (RMM: 4,000,000, Aldrich) mixture are prepared by a standard solvent casting technique used for the preparation of polymer electrolyte films. The compositions are in the form PEOn:GnRH (where n=10 or 20). This represents the molar ratio of the ethylene oxide (EO) repeating unit to GnRH. PEO10:GnRH represents 1 molecule of GnRH associated with 10 EO units. For each preparation, 1 g of PEO is used and the mass of GnRH to be used is calculated by dividing the molecular mass of the GnRH by the molar ratio of 10 and the molecular mass of EO repeat unit (i.e. 44).

The calculated mass of GnRH is then added to 1 g of PEO in 50 mL of distilled water (for hydrophilic molecules) or acetonitrile:ethanol (for hydrophobic molecules) and stirred until complete dissolution. The mixture, which is a viscous solution, is then cast into polystyrene 2 cm diameter culture dishes. Before the polymer has cured, a loop of platinum wire was inserted into the solution such that it is firmly held in place by the cured polymer. The solution is then covered and the solvent is allowed to evaporate at room temperature. The film is then peeled from the well and stored in a sealed plastic bag over silica gel in a desiccator.

### Example 3

### Melatonin

Interstitial Fluid Acquisition Models: The ability to penetrate the skin, and the metabolic changes that occur in the skin, vary from substance to substance: for example coumarin is rapidly absorbed by the skin and passes through the barrier unchanged, while some esters may be totally modified. Permeation of substances through the skin (specifically across the stratum corneum) is a diffusion controlled process where absorption of individual substances are related to lipophilicity (represented by the partition coefficient for an octanol/water) and the molecular weight. The effect of one substance on another must also be taken into account, i.e. in the above example for coumarin absorption it was found that the take up of coumarin was greater from an oil-in-water emulsion than from an ethanolic solution. Thus applicants have determined the following factors are important in skin absorption: degree of hydration of skin, skin temperature, application vehicle, idiosyncratic factors, lipophilicity of materials, volatility of the materials molecular volume of individual components time of contact, concentration of analyte (relationship between sampled dose and absorption is compound and species specific), surface area, degree of skin barrier compromised by skin disease/physical damage etc., age of skin, number of hair follicles/thickness, and skin metabolism of components.

A simplified model can be developed to account for the transport of molecules out of the skin and through the sampling chamber. The model consists of diffusion through the skin, which can be approximated by diffusion through a semi-porous protein matrix membrane and then diffusion into a bulk solution. The concentration profile in the membrane and in the bulk solution may not be consistent, in which case a partition coefficient can be used to relate the transport of molecules from the membrane to the bulk solution. The use of an iontophoretic device can, by nature of the process, produce an electric field. The presence of an electrical charge can enhance or slow down the diffusion of molecules depending on the gradient of the electric field.

The bulk phase diffusivity needs to be adjusted to take into account the winding path through a porous matrix. The effective diffusivity is calculated from the bulk diffusivity, void fraction, and the tortuosity. The mass transfer coefficient can be calculated from the effective diffusivity and the membrane thickness. The flux of molecules in the presence of an electrical charge can be calculated with the Nernst-plank equation.

TABLE 3
Diffusion and mass transfer coefficients calculated for a few
biological molecules in membranes of different thickness.
${D}_{e} = \frac{ɛ}{τ} {D}_{AB} \\ ( Geankoplis , 1993 , pg 412 )$
${k}_{m} = \frac{{D}_{e}}{y}$ $( McCabe Smith , 1993 , pg 861 )$
$J = {D}_{e} [ \frac{d C}{d x} + zC ( \frac{F}{RT} ) \frac{d ψ}{d x} ]$ $( Deen , 1998 , pg 454 )$

| | | mass transfer | | | | | |
|---|---|---|---|---|---|---|---|
| | | coefficient | | 1 mm | .5 mm | 2 mm | 1mm |
| | Diffusivity | microns/s | seconds | min | min | min | hrs |
| Urea | 8.80E-10 | 0.88 | 1136.4 | 18.9 | 4.73 | 75.76 | 1.26 |
| Urea 2.9% gelatin | 6.40E-10 | 0.64 | 1562.5 | 26.0 | 6.51 | 104.17 | 1.74 |
| Urea 5.15% agar | 4.72E-10 | 0.47 | 2127.7 | 35.5 | 8.87 | 141.84 | 2.36 |
| NaCl | 1.51E-09 | 1.51 | 662.3 | 11.0 | 2.76 | 44.15 | 0.74 |
| NaCl 2% agarose | 1.40E-09 | 1.4 | 714.3 | 11.9 | 2.98 | 47.62 | 0.79 |
| KCl | 1.87E-09 | 1.87 | 534.8 | 8.9 | 2.23 | 35.65 | 0.59 |
| KCI porous silica | 6.60E-11 | 0.07 | 14285.7 | 238.1 | 59.52 | 952.38 | 15.87 |

D_{AB} = bulk diffusivity
Dₑ = effective diffusivity
ε = void fraction
τ = tortuosity
kₘ = membrane mass transfer coefficient
y = membrane thickness
J = Concentration flux
Dₑ = Effective diffusivity
C = Chemical concentration
z = Electrical charge
F = Faraday constant
R = Gas constant
T = Temperature
Ψ = Voltage
x = Distance

To illustrate the effect of diffusion through a porous matrix a few calculations were performed as an example. Assuming a protein layer of 1 mm, the mass transfer coefficients were calculated for Urea (0.88 microns/s) and for Urea in a 5.15% agar gel (0.47 microns/s). The addition of a gel matrix decreased the mass transfer by 47%. A change in the concentration at the inside layer of the protein can take longer to reach the sensor side of the layer for thicker membranes and membranes that have a more complex pore pathway. Urea in a 5.15% agar gel can take 35.5 minutes to traverse a membrane by diffusion alone.

Melatonin EIA: Fluid samples were assayed using a commercially available direct saliva melatonin EIA (American Laboratory Products, Cat. No. 001-EK-DSM). This is a competitive binding assay. The samples, controls, and standards are incubated with melatonin biotin conjugate for three hours and a binding competition for a melatonin antibody, which is bound to the microtiter plate, occurs between the melatonin conjugate and the melatonin in the samples. The more melatonin that is present in the sample, the less biotin conjugate is bound. After three hours the plate was washed and enzyme label was added for one hour during which time binding between the conjugate and enzyme occurs. After one hour, the plate was washed and TMB substrate was added. The substrate is converted to a chromophore that absorbs light at 450 nm, in proportion to the amount of enzyme present. The more that light is absorbed indicates that less melatonin was present in the sample. Stop solution is added after a thirty minute incubation, and the plate was read using a BioTek EL800 microplate reader.

RESULTS: The concentrations of melatonin in the samples and controls were computed using the 4-parameter logistic model available in the BioTek KC Junior software. To normalize the data, the concentrations from the pre-melatonin saliva and interstitial fluid samples were subtracted from those obtained after melatonin ingestion. This gave melatonin values that were due solely to melatonin ingestion and removed any background readings due to cross reactivity to other interstitial fluid or saliva components as well as any background measurements due to the matrix of the iontophoresis electrode buffer itself.

Four out of the five volunteers showed an increase in interstitial fluid melatonin after ingestion (mean=9.0+/−6.2 pg/ml). Five out of the five volunteers showed an increase in saliva melatonin ranging from 110.8 to >324 pg/ml. The results are listed in Table 4.

TABLE 4
Comparison of saliva and interstitial fluid (I.S.F.) melatonin
concentrations from the clinical trial samples.

| Volunteer | Saliva Melatonin (pg/ml) | I.S.F. Melatonin (pg/ml) |
|---|---|---|
| 1 | 251.456 | 9.736 |
| 2 | >384 | 1.512 |
| 3 | >384 | — |
| 4 | 174.412 | 8.036 |
| 5 | 101.304 | 16.632 |

To assess and confirm the reliability of the sampling and immunoassay analysis and to correlate to literature values, the pre melatonin saliva values were averaged (n=5, mean=17.5+/−8.4 pg/ml). This compares to approximately 8 pg/ml of melatonin that is normally observed in saliva samples at 8:00 PM, the time that the pre melatonin samples were collected.

### Example 4

### TRH

Thyrotropin-releasing hormone (TRH) is a tripeptide secreted by the hypothalamus and stimulates the pituitary gland to release thyroid stimulating hormone (TSH) and prolactin. TRH deficiency has been found to be responsible for hypothalamic hypothyroidism and can be corrected with oral administration of TRH. Enhanced transport of thyrotropin-releasing hormone (TRH) through excised rabbit pinna skin was achieved by means of iontophoresis with continuous current or monophasic periodically pulsed current. In the transdermal iontophoretic delivery of TRH, the pulsed iontophoretic flux exceeded that obtained with a continuous current. Therefore, this can also be used in conjunction with system of the present invention.

Buserelin is a man-made drug that is used in the treatment of prostate cancer. It is a drug used to enhance and/or replace hormonal therapy. Buserelin reduces the production of luteinizing hormone, leading to a fall in the levels of testosterone, which may result in shrinkage or slowing down of the growth of the cancer cells. Buserelin is delivered in a pulsatile manner, given by injection under the skin three times a day for the first week, and is then continued as a nasal spray six times per day in each nostril. Sometimes people find the injection slightly uncomfortable, and may notice an area of redness at the injection site afterwards. The nasal spray causes temporary irritation to the nasal lining. A comparison of iontophoretic release and passive release of buserelin from hydroxyl-ethylcellulose hydrogel through a cellulose membrane showed matrix release for both. When continuous non-interrupted current with different current densities (0.1-0.3 mA/cm⁻²) was applied, linear dependence of the final cumulative amount of buserelin on current duration and density was observed. Iontophoretic enhancement was also significant for release behavior. Therefore, this can also be used in conjunction with system of the present invention.

Iontophoretic pulsatile transdermal delivery of human parathyroid hormone (hPTH(1-34)) was examined in Sprague-Dawley (SD) rats, hairless rats, and beagle dogs. These findings suggest that this iontophoretic administration system could create a repeated-pulsatile pattern of serum hPTH(1-34) levels without the necessity for frequent injections, and may be useful for the treatment of osteoporosis with hPTH(1-34). Researchers have shown that intermittent administration of PTH(1-37) improves growth and bone mineral density in Uremic rats. Therefore, this can also be used in conjunction with system of the present invention.

Methylphenidate, brand name Ritalin, is a mild CNS stimulant. Methylphenidate is rapidly and extensively absorbed from tablets following oral administration; however, owing to extensive first-pass metabolism, bioavailability is low (approximately 30%) and large individual variation exists (11 to 52%). Noven Pharmaceuticals, Inc. has developed a transdermal methylphenidate patch and is currently awaiting FDA approval. Armaquest Inc. has developed and patented an encapsulated drug for pulsatile delivery of methylphenidate. Therefore, this can also be used in conjunction with system of the present invention.

Mecamylamine is a central nicotinic receptor antagonist that is believed to reduce the rewarding effects of cigarette smoking. Transdermal nicotine/mecamylamine patches are currently being marketed. However, high doses of mecamylamine cause shakiness, dizziness, fainting, constipation, and even convulsions. Furthermore, prior research has suggested that mecamylamine blocks the reinforcing effects of alcohol in animals. A study, published in the May 2002 issue of Alcoholism: Clinical & Experimental Research, has found that mecamylamine reduces the self-reported stimulant and euphoric effects of alcohol in humans, and also decreases their desire to drink more. The system of the present invention would therefore amenable to a dual pulsatile delivery system. In this manner, pulsatile delivery of nicotine followed by, or in conjunction with, low dose pulses of mecamylamine would provide sufficient amounts of nicotine and sufficiently low doses of mecamylamine to treat the addiction yet avoid the side effects that have been reported, thus increasing the efficacy of the cessation regime.

### Example 5

### Lithium

Bipolar disorder, also known as manic depression, afflicts more than 2.3 million American adults according to the National Institute of Mental Health. Because of the great morbidity and mortality rates associated with this illness, long-term treatment is often necessary to prevent the recurrence of manic episodes, reduce the loss of productivity, and control associated medical costs. The most widely used medication for maintenance treatment of bipolar disorder is lithium. Lithium has been shown to cause a prophylactic response in more than two-thirds of patients with bipolar disorder and reduce suicide risk more than eight-fold. Unfortunately, lithium also has a very narrow therapeutic window of effectiveness, with toxic effects at the high end and ineffectiveness at the low end.

Lithium has been shown to cause a prophylactic response in more than two-thirds of patients with bipolar disorder and to reduce suicide risk more than eight-fold. Unfortunately, lithium also has a very narrow therapeutic window, with toxic effects at the high end and ineffectiveness at the low end. Most patients who take lithium experience adverse side-effects, most likely due to the initial, greater than therapeutic levels, which results in poor rates of medication compliance. However, were it possible to maintain serum lithium levels within the narrow therapeutic window, the risk of toxicity and the occurrence of side-effects would be greatly reduced, and patient compliance would increase significantly.

There are two primary factors that lead to the wide fluctuations of plasma concentration of lithium, and most other drugs that are administered using conventional delivery methods. First, is the lack of controlled delivery of the drug to maintain constant plasma concentrations in the body. Physical activities and metabolic variation from individual to individual result in greatly increased or decreased rate of drug uptake, thus further reducing the effectiveness of an oral medication taken on a regular basis. Second, is the infrequency with which the drug is administered. Lithium, for example, is typically taken in pill form three times per day. After taking a pill, the plasma concentration increases significantly, followed by a steady decay until the next pill is taken. To overcome these problems, it is necessary to both acquire feedback from the body on a regular basis and to administer the drug on an as-needed basis to maintain the desired plasma concentration level. It seems obvious that conventional methods of delivery, such as pills, cannot address these issues. Using the fluid analyzing system, lithium can be effectively delivered transdermally using iontophoresis.

Currently, the only method available for monitoring blood lithium levels is by performing occasional laboratory tests, the results of which take several days to obtain. Between these periodic tests, daily lithium fluctuations in blood serum levels go unchecked, increasing the risk of toxicity and the development of intolerable side effects. The side effects of lithium are a major factor in non-compliance and contribute to its decreased usage in the United States. More than 80% of patients who are prescribed lithium experience some adverse effects, including weight gain, nausea, tremor, reduced sexual drive or performance, anxiety, hair loss, movement problems, and/or dry mouth. Furthermore, advanced stages of toxicity are generally not addressed until the patient verbally complains of them. By achieving a stable therapeutic response, the risk of side effects and toxicity could be greatly reduced or avoided, and patient compliance would increase significantly.

The present invention provides an automated, non-invasive lithium delivery and monitoring system (LDMS) that provides precise dose delivery and simultaneous monitoring of lithium in order to maintain optimum therapeutic levels. Utilization of this high-precision, closed-loop system alleviates many of the problems associated with lithium-treated bipolar disorder, including side effects, risk of toxicity, and non-compliance.

The LDMS has the potential to vastly increase patient compliance by reducing the side effects, improving the quality of life of patients by relieving them of the manic highs and depressive lows, and significantly reducing the associated financial burdens on the healthcare industry by decreasing the number of suicides and the related medical costs due to non-compliance. This is made possible due to the LDMS' ability to maintain constant therapeutic concentrations of lithium and/or other anti-psychotic medications, thereby eliminating unnecessary complications due to inappropriate dosages. While the LDMS is not a cure for bipolar disorder, it offers the greatest promise and one of the best means of controlling its debilitating symptoms and enabling those who suffer from it to lead more normal and productive lives.

To test the iontophoresis system, platinum electrodes were constructed by soldering 125 μm diameter platinum wire to insulated wire. The first electrode (the positive electrode) was positioned and secured into the donor compartment, within 1 mm of the bottom of the compartment, taking care not to touch the bottom and present the possibility of puncturing the skin sample after insertion. The second electrode (the anode) was secured to the outside of the device flush to the bottom of the chamber. The fastening screws and nuts extended beyond the bottom of the chamber, acting as legs that elevated the device a few millimeters above the culture plate. This ensured that the receiver compartment solution covered the electrode at all times during the experiment to maintain continuity between the cathode and anode.

EpiDerm culture samples (Model EPI-212 kit, 8 mm diameter) contained in their inserts, were obtained from MatTek and placed into the Millicell device. The assembly was equilibrated to 37 C for 15 minutes. The lithium solution was then transferred into the donor compartment and onto the stratum corneum (top layer of EpiDerm) and readings were taken at 0, 5.0, 10.0, 15.0, and 20.0 minutes to monitor the time course of lithium delivery. Between samplings, a transfer pipette was used to constantly agitate and mix the receiver solution to ensure that the lithium diffused into the receiver solution evenly.

Two lithium carbonate concentrations were tested as donor compartment solutions, 52.8 mM and 105.6 mM. These concentrations were chosen using chemical engineering mass transfer modeling to determine the maximum amount of lithium that would have to be delivered to a large human being such therapeutic plasma levels can be achieved. In this manner, it was assured that there would be sufficient lithium in the donor compartment for the analysis and to provide evidence as to whether such high concentrations of lithium carbonate (donor compartment) would transfer through dermis in an uncontrolled manner.

In each experiment, three chambers were prepared and connected to three Iomed Phoresor iontophoresis systems. The chambers were placed in a 6-well culture plate and 5.0 ml of Hanks balanced salt solution was added to the three wells. The plate was placed on a test tube rack in a 37° C. water bath to maintain temperature throughout the experiment. At each time point, 50 μl of the receiver solution was removed, sealed in a 12 mm×75 mm test tube, and placed aside for lithium concentration determination. At the end of the experiment, all receiver samples were assayed for lithium concentration using a commercial lithium assay. The reduction in volume after each 50 μl sampling was taken into account and the receiver compartment lithium concentration was computed.

Lithium assay: To determine the amount of lithium delivered through the artificial skin, samples were assayed using the ThemoTrace lithium assay kit, containing lithium reagent (cat. no. TR66056) and 1.0 mM lithium standard (cat. no. TR66901). In this one standard assay, the reagent blank is subtracted from all samples. The concentration is computed by taking the ratio of sample absorbance to standard absorbance and multiplying by the concentration of the standard. The assay was read at a wavelength of 515 nm using a BioTek 800 microtiter plate reader.

Results: The time course of delivery was plotted for two different lithium concentrations at two different current dosages. A two-fold increase in current caused an equivalent increase in the amount of lithium delivered. Likewise, a two-fold increase in the donor compartment lithium carbonate concentration caused an equivalent increase in the receiver compartment lithium concentration. This was repeated, and analyzed statistically for relevance.

These data indicate that lithium is transported across artificial skin in proportion to the amount of current delivered, as well as to the amount of lithium present in the donor compartment.

In the control studies, in which electrodes were present but no current was passed, there was no passive delivery of lithium without iontophoresis.

Develop plant/system models and develop a closed-loop control system: That which differentiates the Lithium Monitoring and Delivery System (LMDS) from other drug delivery systems is the inclusion of a control system, which delivers only the amount of lithium required to maintain the desired plasma concentration

For use in a device such as the LMDS, there are two aspects of control system development that need to be considered. The first is the mathematical equations (models) that are incorporated into the controller. The second is the hardware platform upon which the control algorithms are executed.

An automatic control system has four fundamental components: inputs, outputs, the controller, and the plant. The system output tracks the system inputs in a robust manner (in the context of control systems, robustness refers to the ability to have the output track the input when presented with noisy inputs and inaccurate models). In order to achieve this objective, a model of the plant is developed and a controller is developed using any one of numerous established techniques that provides the necessary system performance.

The advantage of such a model, as compared to more simple, linear control models, is that this model provides the means for “canceling” the non-linearities inherent in the underlying plant. For this system, the non-linear model includes the temporal delay between the delivery of lithium at the specific site on the skin, uptake distribution into the blood stream, and the temporal delay between lithium entering the blood stream and when it has diffused back into the interstitial fluid. Based upon the previous experiences measuring glucose, it was found that there was approximately a five minute delay between concentration levels in the blood stream and interstitial fluid. Lithium diffuses faster and is available to the tissue with less delay due to its size and charge.

Since one of the primary rationales for this technology is to limit the overshoot of serum lithium concentration that occurs when pills are consumed, simply treating the body (plant) as a first order system would be insufficient. Instead, the plant is modeled as at least a second-order system, thereby providing the ability to use “velocity” feedback control, which is known to be effective at controlling overshoot. (In the context of this invention, velocity is the rate of change of serum lithium concentration).

Two factors that enhance the likelihood of success are that the plant (body) is “well-behaved” and that the time constants involved are quite long. Unlike many mechatronic systems, the human body's dynamics, with regard to lithium regulation, is fundamentally linear. With linear systems, small input changes lead to small output changes, making such systems inherently easier to control than those that behave in a non-linear fashion. Combined with the long time constants, the controller is able to regulate plasma lithium concentrations in an extremely precise manner.

As mentioned above, the control algorithm needs a platform on which to run. Design considerations for this platform include the number of bits for the input A/D conversion, the number of bits for the output D/A conversion, and the number of processor bits, available memory, etc. For the present invention, having eight bits of A/D and D/A provide adequate accuracy while being inexpensive and straightforward to manufacture. On the input side, eight bits allow discrimination of differences in plasma concentration of less than 0.01 mM and output current discrimination of 0.015 mA. The calculations required for this controller are relatively few, and combined with the long time constants (i.e., long durations between output updates), can be performed on very meager systems. Serum lithium concentrations are controllable to within 0.05 mM from the nominal set-point under all conditions.

Produce iontophoresis delivery system: There are three issues that need to be addressed: the size of the transdermal delivery (agent delivery system) patch, the design of the iontophoresis electronics, and the design of the actual patch itself. Before detailing the efforts to address these three issues, serum lithium dynamics are first discussed as they have a major impact on the first issue.

Serum lithium dynamics: The therapeutic level of lithium in the bloodstream is between 0.8-1.2 mM. Research indicates that the plasma elimination half-life ranges from 12-27 hours, with even longer times for elderly patients and chronic lithium users. From this information, the dose rate of lithium required to maintain the therapeutic concentration and the amount needed to be stored to provide a full day's supply can be calculated.

To determine the amount of lithium that needs to be delivered, an exponential decay is assumed. This allows calculating a time constant of −0.028 t−1, where t is expressed in hours. Using this time constant, the amount of time for the plasma concentration to decay from the high end to the low end of the therapeutic range is 14.0 hours. To replenish the 0.4 mM requires the addition of 2.0 mmoles (12 mg) of lithium per 14.0 hours, or 0.14 mmoles/hr (0.8 mg/hr). Thus, the amount of lithium needed for a full day's supply is approximately 3.4 mmoles (21 mg).

Patch size: The two factors that determine the patch 74′″ size are the amount of lithium that needs to be stored and the maximum FDA allowable current for iontophoresis applications. It is also necessary to demonstrate that the requisite amount of lithium can be delivered while complying with FDA regulations.

FDA regulations limit iontophoresis current to a maximum of 4 mA. Assuming the system requires the maximum allowable current, and knowing that currents above 0.25 mA/cm2 can cause irritation to the skin, a patch size of 16 cm² is anticipated. This is slightly smaller than a BandAid™ Tough-Strips™ bandage.

As previously stated, the amount of lithium needed per day is approximately 21 mg. Since the solubility of lithium chloride (MW=42.39 g/mole) is 769 g/liter (18 Molar) it is possible to store the required amount of lithium in under 1 ml. Using a lower concentration, for example, it is possible to be able to achieve a concentration of 4M in a gel, the quantity of lithium carbonate to store is approximately 1 ml. Assuming this is evenly spread across the patch, its thickness would only be 0.5 mm.

To determine the amount of lithium that can be iontophoretically delivered to the patient (swine), the total charge delivered to the animal, using the maximum FDA allowable current of 4 mA, is calculated.

Q=It 14.4 C/hr

Since lithium has a charge of +1, and assuming that the lithium carries all of the charge, the number of atoms of lithium delivered per hour is found to be 1×1020 ions. Since the molecular weight of lithium is 6, the weight of lithium transported per hour is 1 mg/hr. This exceeds the required dosage by 20%.
Monitor Peak and Duration Levels Vs. IM Levels

In-vivo delivery of lithium in mice: To compare theoretical vs. actual delivery in the in vivo hairless mouse model, charge delivery models (current models) were used to calculate the concentration of lithium that theoretically would be delivered to the animal based upon iontophoresis time, current, and lithium concentration in the delivery chamber.

To determine the percentage of charge carried by the lithium, first the amount of lithium was calculated that would be expected to be found in the mice. First, the total charge delivered to the animal during the course of the experiment was determined.

Q=It

Q=1(mA)×1/1000(A/mA)×20(min)×60(sec/min)

Q=1.2 A sec

Q=1.2 C

Next, knowing that lithium has a charge of +1, the number of atoms of lithium delivered to the animal can be calculated.

e=1.6×10⁻¹⁹ C/electron

Atoms=Q/e

Atoms=7.5×10¹⁸

N_{A}=6.02×10²³ atoms/mol

1.25×10⁻⁵ mol

Finally, knowing the volume of blood in a mouse, 0.005 liters, the maximum lithium concentration that would be delivered, 2.49 mM, can be estimated assuming all of the charge is carried by the lithium. The lithium carries only about 10% of the total charge delivered to the mice.

The animal model employed was that of the SKH-1 male mouse, 6 weeks old, obtained from Charles River Laboratories. The mice arrived the morning of the experiments and were used within three hours of their arrival.

Due to the fragility of the iontophoresis pad application and electrode attachment, the mice had to be restrained during the experiments. To accomplish this, a commercial mouse restrainer was purchased from Kent Scientific and modified with two holders on either side of the restrainer. The holders were placed over existing access holes in the restrainer such that spring loaded electrodes could be positioned within the holder.

The mice were given a low dosage of Halothane to allow them to be weighed and positioned in the restrainer so that there was minimal risk of the electrode pads being kicked or scraped off of the patch as the mouse was being positioned. The Halothane was administered by saturating a paper towel that had been placed at the bottom of a glass desiccator jar. The jar was placed in an exhaust fume hood prior to opening the Halothane bottle. This gave a saturated Halothane environment, in a well-ventilated area, and allowed the effects of the anesthesia to be closely monitored. The mouse was placed into the jar for approximately 15 seconds and was removed immediately after succumbing to the anesthesia. This provided enough sedation to allow the mouse to be handled and placed into the restrainer without providing undue stress. The animals recovered completely within two minutes of being placed in the restrainer.

The mouse was positioned such that the iontophoresis electrodes are on either side of the rump area. In this manner, the current does not flow through vital organs. After positioning, the electrodes were connected to the Phoresor II iontophoresis system. A current dosage of 20.0 mA min was applied for a period of approximately 20 minutes.

At the end of the treatment, the mice were removed from the restrainer and decapitated using a rodent guillotine from Kent Scientific (cat. no. DCAP). Trunk blood was collected into a funnel on top of 15 ml conical tubes. After ten minutes, to allow the blood to clot, the tubes were spun at 2000 g for 15 minutes. The serum supernatant was aspirated, placed into 1.5 ml conical tubes and spun again to remove any remaining blood cells. The serum was transferred to clean 1.5 ml conical tubes and the assay was performed.

The experiments were run using five treatment groups, with n=3: First; control using passive diffusion, Second; iontophoresis with 52.8 mM lithium, Third, iontophoresis using 158.4 mM lithium, Fourth; I.M. injection of 6.76 mM lithium in restrained mice, and Fifth, I.M. injection of 6.76 mM lithium in mice that were not restrained (“mobile”).

Lithium carried only about 10% of the total charge delivered to the mice. However, the lithium was diluted into a 0.9% NaCl solution and therefore the sodium ions were carrying a large portion of the charge. By eliminating the sodium ions, lithium ion transport is subsequently increased. Using a lithium concentration of 158.4 mM resulted in serum concentrations of approximately 0.2 mM using cathodal iontophoresis of 0.2 mA*min.

Iontophoresis electronics: Included in the electronics are a DC-DC voltage source to increase the voltage, a constant current source to deliver current to the individual, and protection circuitry to limit the current to the individual in the case of catastrophic failure.

Patient protection circuitry is used to shunt any excess current to the LMDS eliminating any possibility that the circuitry can “shock” a patient. Using a resistor to monitor the current and a Zener diode to shunt current if it exceeds a threshold value allows the system to produce a current near the FDA maximum value, while discharging the current if it exceeds the recommended value.

The patch itself can be produced by casting the lithium carbonate into a hydrogel at a 4M concentration. Prior to curing, the platinum electrode is incorporated into the gel, providing excellent electrical connection to the delivery solution. Finally, hydrophobic adhesive is applied to the bottom of the gel. This eliminates any diffusion of the lithium into the patient without current applied. The electrode can be mated to the circuitry through a standardized connection such as a flip chip connection. The first device is approximately the size of a hand-held computer, however the final device can be considerably smaller so it can be comfortably worn for extended periods of time.

Paired Student's t-Test: The student's t-test was used to test the null hypothesis of no significant difference between two groups of data and to determine if the obtained results provide a reason to reject the hypothesis that they are merely a product of chance factors.

TABLE 1
Statistical analysis summary of in vivo lithium
delivery in mice, using the student's t-test

| | | Degrees of | |
|---|---|---|---|
| Pairs | t-Stat | freedom | t-Prob |
| Control vs 26.4 mM | −17.7 | 2 | 0.003 |
| Control vs 79.2 mM | −16.3 | 2 | 0.004 |
| Restrained vs Mobile | −1.07 | 2 | 0.397 |

Table 1 clearly demonstrates a significant difference (p < 0.01) between passive delivery of lithium and iontophoresis assisted delivery. The results also demonstrate an increase in lithium delivery in proportion to the concentration of lithium in the delivery pad. There was no significant difference between the restrained versus mobile groups as evident from the large variation of the restrained group.

Again, it must be noted that, as observed in the in vitro studies, there was a statistically significant iontophoresis current and donor dose dependency on delivery of lithium carbonate observed in the results for both artificial human skin and nude mice.

The present invention was able to deliver sufficient amounts of lithium carbonate, transdermally, in a non-invasive and controlled manner to an in vitro human skin model and an in vivo animal model, the nude mouse. Very low concentrations of lithium were delivered passively, providing almost no delivery during the electrically “off” state. Transdermal drug delivery is capable of controlled delivery of sufficient quantities of lithium carbonate to maintain therapeutic levels in humans.

Small pulses of lithium can be delivered throughout the day based on plasma sample measurements, again obtained non-invasively using transdermal methods. Only during initial administration is a large concentration to be delivered, then the embedded closed-loop logic system can function such that lithium levels are monitored regularly, and maintained at a constant therapeutic level of approximately 1 mM. A closed-loop delivery system improves maintenance of therapeutic levels, adjusts for activity level, adjusts for food and water intake and therefore decreases the undesirable side effects (caused by the large plasma level fluctuations) that cause a great percentage of patient non-compliance.

### Example 5

### Nicotine

Current transdermal patches deliver nicotine in a passive manner and are not capable of pulsatile delivery. Nicotine gum, inhalation devices and lozenges deliver nicotine in much the same manner. The nicotine spray delivers a pulse of nicotine that resembles the same delivery pattern as that of smoking a cigarette, but can only deliver half the amount of nicotine. Decreasing the dosage of spray during a smoking cessation regimen requires a different formulation of spray, containing smaller and smaller amounts of nicotine. This complicates the ability to deliver serially decreasing doses of nicotine as are typically utilized in addiction withdrawal programs. In addition, since the rate of delivery is completely controlled by the patient, it is possible that the spray can be over-used.

Current nicotine delivery patches rely on the passive diffusion of nicotine through the skin and into the fluid that surrounds the cells beneath the skin (interstitial fluid). From there, the nicotine diffuses into the capillary network, enters the blood stream, and is delivered to the brain. The nicotine is contained in a textile fiber material within the patch and nicotine is delivered continuously, as long as the patch is worn. This method of delivery fails to mimic plasma nicotine levels produced by cigarette smoking since it is not pulsatile and does not deliver the same level of nicotine.

The use of passive diffusion nicotine patches as part of a smoking cessation regimen has proven to be ineffective. In fact, no advantage for nicotine replacement therapy (NRT) was observed in either the short or long term for nearly 60% of California smokers classified as light smokers (<15 cigarettes/day). Since becoming available over the counter, NRT appears no longer effective in increasing long-term successful cessation in California smokers.

The agent delivery system, with incorporated microfluidic pumps and valves, provides the capability to deliver nicotine in a truly pulsatile manner by a less than 2 cm² patch. By means of the microfluidic pumps and miniature reservoirs, various levels of nicotine can be introduced into the reservoirs for iontophoretic transdermally delivery. The “on state” can be followed by an “off state” wherein the nicotine is completely emptied from the reservoir and replaced with normal saline, or left empty, and the iontophoresis electrode is turned off.

In this manner, true square-wave pulses of nicotine can be delivered. Unlike current transdermal nicotine patches, which do not have the capacity to remove the nicotine from the system other than by removing the patch, the agent delivery system is fully automated, programmable, and can deliver nicotine in a pulsatile manner. The nicotine pulses can be continuously decreased during the entire cessation regimen. Since the plasma nicotine profile more closely resembles that obtained while smoking a cigarette, the agent delivery system is more effective, thus increasing the likelihood that the full cessation regimen can be followed.

The agent delivery system can be worn for one day during waking hours (removed at night, applied in the morning). Depending on the most effective cessation regimen, a series of agent delivery systems can be manufactured with serially decreasing dosages of nicotine. The “Day 1” delivery dosage for each pulse can be automatically decreased by a minimal amount throughout the day with the ending dose being equal to the starting dose of the following day “Day 2” agent delivery system, thus providing the ability to slowly and serially decrease the nicotine dosage throughout the treatment period. The interval between delivery of nicotine can also be modulated throughout the day.

The storage volume of the nicotine solution is not limited to the 120 μl volume of the reservoir. Soft polymer PDMS reservoirs can be constructed and bonded to the silicon chip to easily provide 1.0-2.0 ml storage volumes. With an initial nicotine concentration of 50 mg/ml (maximum solubility), the 20 μl membrane interface chamber can contain 1 mg of nicotine. The membrane interface chamber is continuously replenished during the pulse period using the microfluidic pumps, thereby providing a constant concentration of nicotine in the membrane interface chamber. In this manner, current and time are the limiting variables. For example, a pumping rate of 20 μl per minute can make 5 mg available for delivery within a five minute pulse, thereby requiring only 20% delivery efficiency to equal the required 1 mg dose. A storage volume of 2.0 ml can supply sufficient nicotine for at least 25 five minute pulses, or 50 two and a half minute pulses (truly any combination or permutation) to be delivered throughout the day.

The membrane interface chamber can be emptied and filled with an isotonic buffer or saline solution between pulses. The entire patch can be covered with a backing layer of polyester film, which also houses a battery, similar to existing passive dermal patches. The nicotine solution can also be used with an electrolyte polymer membrane as described above that can prevent “leakage” both within and outside the patch. The electrolyte polymer membrane can be stimulated by electrodes to release the nicotine solution in pulses.

Cyclic voltammagrams indicted that nicotine is oxidized at voltages approaching 1 volt. The half-cell containing nicotine can be kept at a potential below 0.7V.

Polymer matrix electrolytes have been shown to be ideal for storage and delivery of molecules, such as lithium and lidocaine using iontophoresis. Polymer electrolytes are solid-like materials formed by dispersing nicotine in a high molecular weight polymer. In essence, the molecule is trapped within the polymer until the application of an electric current. Application of electric current causes the porosity of the polymer to increase, hence providing controlled release of nicotine. This technology allows molecular concentrations as high as 4M to be incorporated into the matrix. The use of polymer electrolytes to deliver nicotine can simplify the agent delivery system considerably since it can eliminate the need for reservoir and pumps. CMOS circuitry can control the amplitude and duration of the nicotine transfer in order to deliver precise amounts of nicotine. This can also provide a secondary fail-safe mechanism in case of trauma to the patch, or failure mode operation since transdermal delivery of nicotine only occurs when current is applied.

Polymer electrolytes are ionically conducting polymers that are composed essentially of solutions of ionic salts in heteropolymers, such as poly(ethylene oxide) (PEO). PEO is a semicrystalline solid with a high proportion of crystalline regions distributed in a continuous amorphous phase, which means the PEO is a solid at room temperature (tm=65 C and Tg=−60° C., thus it has structural integrity) and the PEO chains in the amorphous regions have a sufficient degree of segmental mobility, permitting ion transport. The amount and state of amorphous regions of polymer is therefore crucial to its functioning as a polymer electrolyte, which can be altered by many factors, including the type and amount of added ions (including medicinal drugs) and the method by which the polymer electrolyte is formed.

As its low molecular weight analogs, the poly(ethylene glycol)s, the PEO has minimal adverse reactions to skin (skin irritation and sensitization), as well as a sufficient loading capacity of drug dose. Unlike its low molecular weight analog like poly(ethylene glycol), which tends to form liquid or semisolids, PEO forms a solid matrix. The drug delivery property of the polymer electrolyte film for iontophoresis is assessed by checking its AC impedance.

PEO-salt complexes can be formed as soft, flexible films with a thickness that can vary from a few micrometers to about 100 micrometers. Previous studies showed that PEO can incorporate large concentrations (˜4M) of salt, making it eminently suitable as a matrix into which highly potent drugs may be incorporated.

Preparation of polymer-nicotine films: Films of PEO (RMM: 4,000,000, Aldrich) mixture are prepared by a standard solvent casting technique used for the preparation of polymer electrolyte films. The compositions are in the form PEOn:salt (where n=10 or 20). This represents the molar ratio of the ethylene oxide (EO) repeating unit to the salt. PEO10:salt represents 1 molecule of salt associated with 10 EO units. For each preparation, 1 g of PEO is used and the mass of salt to be used is calculated by dividing the molecular mass of the salt by the molar ratio of 10 and the molecular mass of EO repeat unit (i.e. 44).

The calculated mass of salt is then added to the 1 g of PEO in 50 mL of distilled water and stirred until complete dissolution. The mixture, which is a viscous solution, is then cast into polystyrene culture dishes (1-2 cm diameter). The solution is then covered and water is allowed to evaporate at a room temperature. The film is then peeled from the well and stored in a sealed plastic bag over silica gel in a desiccator.

The film can be tested by applying it to a cadaver skin sample mounted in the diffusion cell. The same scheme of pulse patterns can be used to determine delivery efficiency. The receptor compartment solution can be sampled and analyzed using EIA analysis and TLC to determine the electrochemical stability of nicotine using this delivery methodology.

### Examples

### Category 2

The present invention may also be configured to be a stand-alone delivery or a stand-along monitoring device. Monitoring may be accomplished by utilizing the feedback unit to monitor on or more agents in the interstitial fluid extracted from the patient. The feedback unit can be programmed to extract a sample for analysis. The feedback unit may be configured to analyze the interstitial fluid for more than one target molecule. The sensors used to detect the presence or absence of a target molecule may be integrated into the data storage unit where sample results may be stored for retrieval with an external computer system.

The examples provided for the stand-alone monitoring category involves: an integrated circuit programmed to sample the patient based upon a fixed time interval or on demand. A monitoring device to sample the patient utilizing a reverse iontophoretic method. Reagent, reaction and waste chambers and/or reservoirs and a microfluidic system to transport the fluids between reservoirs. The unit has an integrated USB port and may be adapted for wireless signal transmission.

The present invention for this category may be utilized, but not limited to non-invasive monitoring of: glucose, biological markers such as blood electrolytes, blood ions, biologically active substances, pharmacological drugs, drugs of abuse, pesticides, antibodies, hormones, etc. These examples are for illustrative purposes and intended to be descriptive rather than limitations.

### Example 1

### Glucose

Using interstitial fluid withdrawn from a subject, glucose concentration was measured using microscopic glucose sensors prepared from Teflon coated platinum wire measuring 125 μm in diameter. After attaching the platinum wire to a sensor stalk, a glucose oxidase membrane was applied to the electrode. The electrode was first dipped for 10 seconds in a cellulose acetate solution containing 1 g cellulose acetate: 24 g cyclohexanone: 24 g acetone. After drying for 1 minute, the electrode was dipped in a glucose oxidase (GOD)/bovine serum albumin (BSA) solution containing 0.5 ml of GOD (185 IU/mg/ml) and 0.5 ml of BSA solution containing 50 mg/ml BSA. Both solutions were prepared in 0.1M phosphate buffer, pH 7.4. Finally, after drying for 1 minute, the electrode was dipped in a 1% glutaraldehyde solution to promote crosslinking of the proteins. The electrode was allowed to dry overnight at 20 C.

Cyclic voltammetry was performed using the microscopic glucose electrode at +/−900 mv, 250 mHz cycles, with oxidatively derived current flow captured at 425 mv versus a silver/silver chloride reference electrode. Cyclic voltammetry was able to detect glucose over the entire range of physiological and subphysiological concentrations, proving that it is an appropriate technique for monitoring glucose at a wide range of concentrations. A linear regression analysis was performed on the data using a linear-log plot with a high degree of correlation (R²=0.9979) for the mean of three separate measurements over the concentration range. There are several advantages to using cyclic voltammetry to assay glucose as opposed to traditional chemical-based methods. These include the rapidity of detection and quantification (seconds), the sensitivity for glucose, the limit of detection for glucose, and the ability to recycle the reaction (i.e. perform serially repeated assays for days).

During the initial testing of the micro-fluidic PDMS sampling chambers, problems were encountered due the hydrophobic nature of the PDMS material. The hydrophobicity of the PDMS material caused air bubbles to become trapped in the micro-fluidic sampling chamber, and produced uneven flow through the chambers. To remedy this problem, it is necessary to modify the surface of the PDMS to make the material more hydrophilic. Several methods for surface modification were detailed in the previous report, and testing began on two of these methods this month: addition of surfactant to the uncured PDMS material and exposure of the PDMS to HCl.

Previously it was determined that after exposure to high concentrations of HCl for extended periods of time, the material went from hydrophilic back to hydrophobic. The surface modification testing continued using a lower concentration of HCl (0.01M). The water contact angle (as described in the previous report) only reduced to an average of 83 degrees that is only slightly hydrophilic. The goal for acceptable surface treatment was determined to be an angle of less than 65 degrees.

Other techniques for PDMS surface modification were examined. The potential techniques included UV exposure and plasma oxidation.

Optimal redox peak potential selection: In order to choose the appropriate voltage to achieve a peak current, the reduction and oxidation peaks were studied with cyclic voltammetry (CV). The potential was scanned from −0.3 V to +0.6 V vs. Ag/AgCl reference electrode. A very slow scan rate of 0.01 V/s was employed in this study to reduce the charging current associated with faster scans. A PBS buffer solution (pH 7.4) with no glucose and a 70 mM glucose solution were tested and two cyclic voltagrams were compared. From this graph, the oxidation peak is determined to be at +0.3 V.

Scan Rate: The scan rate of the CV influences the accuracy of peak current of cyclic voltagram. Faster scan rates exhibit higher charging effects and therefore reduce the accuracy of the measured peak current. AST examined this influence by testing a glucose solution (70 mM) with different scan rates. As can be seen from, the higher scan rate results in a high charging effect, which interferes with the accuracy of the test. With the slower scan rate, the peak (maximum) current change can be clearly observed at a redox potential of +0.3 V. This peak potential (glucose oxidation peak potential) can be used for all subsequent tests, assuming there is not significant interference at this potential due to interfering molecules (see future work).

Applicants succeeded in identifying and acquiring an appropriate silicone material from which the chambers can be constructed. This biomedical grade silicone material is Dow Corning MDX4-4210, which is a two part catalyst cured silastic with a 10:1 mixing ratio. Tests were conducted to determine the efficiency with which the cured silicone material is released from the mold.

Applicants produced a small number of sampling chambers. To accomplish this, Applicants first produced a set of molds at The University of Michigan Solid-State Electronics Laboratory. The molds were produced on four inch diameter silicon wafers utilizing thick photoresist. SU-8-25 and SU-8-75 photoresists were utilized to produce the 45 μm and the 85 μm thick molds respectively. The geometrical patterns of the silicone chambers are the same as the patterns of earlier glass chambers.

First, the micro-heaters were fabricated on a silicon wafer on top of a MEMS based thick silicon oxide (50 μm) fabrication technology, which acts as the thermal isolation layer. Second, about 50 μm thick PDMS (Dow Corning Sylgard 184) is patterned and cured to form a water container. Finally, a water drop is deposited into the water container and a cured PDMS film (Nusil MED10-6605), with an approximate thickness of 25 μm, is bonded on top of PDMS water container to physically seal the water into the container.

The mechanism of pumping and valving can be explained in the following way: the membrane is actuated (pop-up) by vaporizing water that expands and forces the thin PDMS membrane to actuate. This occurs when there is enough electrical power applied to the micro-heaters. As this membrane actuates, it occludes a conduit and solution can be forced in a particular direction. The figure shows optical microscopic pictures of the comparison between the un-actuated and actuated membrane. The input voltage is 15 Volts, and actuation frequency was 25 Hz.

With an anticipated utilization of sampling every 5 minutes, requiring approximately 20 seconds to pump the solution from the chamber requires a total on time of 3.2 hours/day. The pump was worked for more than six hours without degradation. This six hour test therefore constitutes two days of usage, while the intended lifetime of the device is only one day.

Design the sensor and actuator control circuitry: One of the major circuit blocks is the iontophoresis circuitry. Several sub-blocks are necessary for this circuit. First, the circuit must deliver a constant current independent of the resistance of the skin. Second, the design must utilize a low voltage power source, preferably 5V. Finally, protection circuitry for the patient must be included so excessive currents can not be delivered to the patient.

An initial analysis of the sensor circuit was completed to determine the amount of current necessary and the voltage required to drive that current.

To achieve a measurable dose, a 2 ml sample utilizing a 4 cm diameter patch requires a current of 4 mA for 10 minutes to affect complete delivery. The force of this current is enough to drag neutral molecules, such as glucose, through the skin. It is therefore necessary to maintain this constant current density.

Current density is defined as current per unit area (amps per square meter). Common units are amps per square meter (A m⁻²) or milliamps per square centimeter (mA cm⁻²).

J=I/A (1)

R=ρ(L/A)=Rs(L/W) (2)

V=IR (3)

Where Rs is the sheet resistance in Ω/square and ρ is the resistivity.
The diameter of the larger electrode utilized in Phase I is 4 cm.
Area, A_{big}=(3.14129)*r²=(3.14129)*(2 cm)²=12.57 cm²=1257 mm²
The smaller electrode is slightly irregular, however it can be approximated by a rectangle and entry and exit areas.
Area, Aₛₘₐₗₗ=6 mm×2 mm+entry and exit area=12.5 mm²
This gives an approximate area of:

$$100 ( {A}_{small} ) = {A}_{big} \qquad \text{-(4)}$$

Equating current densities in the larger and smaller electrode and using Equation (1)

$${J}_{small} = {J}_{big} \\ {I}_{small} / {A}_{small} = {I}_{big} / {A}_{big} \\ {I}_{small} = {I}_{big} ( {A}_{big} / 100 ) / {A}_{big} \\ {I}_{small} = {I}_{big} / 100 \qquad \text{-(5)}$$

Therefore to achieve the same current density in the small chamber requires 1/100 the current as was required for the large electrodes. For the example above, this means that Applicants need to deliver 40 μA of current to maintain the current density.

However, with a smaller skin surface area the resistance of the system increases. With resistivity (per unit area) ρ and the length (thickness of the skin) L through the skin remaining constant and using equation (2),

$$ρ L = RA \\ {R}_{small} {A}_{small} = {R}_{big} {A}_{big} \\ {R}_{small} = {R}_{big} {A}_{big} / ( {A}_{big} / 100 ) \\ {R}_{small} = 100 {R}_{big} \qquad \text{-(6)}$$

Using equation (3) the resistance of the skin is 100 times greater for the small sampling chamber than for the large chamber.

$${V}_{small} = {I}_{small} {R}_{small} \\ {V}_{small} = ( {I}_{big} / 100 ) * ( 100 {R}_{big} ) \\ {V}_{small} = {I}_{big} {R}_{big} \qquad \text{-(7)}$$

From this it was determined that to maintain the same current density in the small chamber, 100 times less current is necessary, and to drive the current, the exact same voltage is necessary. Example: Large skin resistance is 10 KΩ and one wants to deliver 4 mA, this requires 40V. For the same skin using a smaller chamber, skin resistance is 1MΩ and current is 40 μA that also requires 40V.

To achieve this increased voltage to drive the constant current requires a special circuit to increase the DC voltage from 5V to 40V in order to maintain a constant current to the individual.

One of the more challenging aspects of the design is providing the high voltage (40VDC) necessary for iontophoresis. In order to develop circuitry that provides this voltage at the required current from a small watch battery with a voltage of 5V, two basic methods can be employed: a step-up transformer or a transformer-less DC-DC converter.

A transformer can be utilized to produce a higher output voltage, with lower operating duty cycle from a low AC voltage source that is produced from a DC source via a DC-AC integrated circuit. Transformer leverage can improve power density and efficiency, reduce ripple, and allow the use of smaller, cheaper integrated circuits. However, they suffer from three types of efficiency loss: transformer/inductor DC resistance and switch resistance losses, transformer-leakage inductance losses, and diode delay losses when the diode is quickly and heavily reverse biased. In addition, transformers are typically costly and microscopic off-the-shelf transformers are not as readily available as other methods of DC-DC conversions.

A transformer-less DC-DC converter is an electronic device used to efficiently change DC voltage from one voltage level to another. They are needed because, unlike AC, DC cannot simply be stepped up or down using a transformer. In many ways, a DC-DC converter is the DC equivalent of a transformer. There are many different types of DC-DC converter, each of which tends to be better suited for particular types of application than for others. Two types of DC-DC converters, best suited for this application, are the charge pump voltage converter and the step-up (switching) voltage regulator.

A charge pump voltage converter typically depends on storing energy in the magnetic field of an inductor. However, this converter can also be implemented by storing energy as electric charge in a capacitor, which reduces the cost of the system. These capacitive charge-pump voltage converters use ceramic or electrolytic capacitors to store the energy and pump the voltage to a higher value. Although capacitors are more common and less expensive than the coils used in other types of DC-DC converters, capacitors cannot change their voltage level abruptly. A charging capacitor voltage always follows an exponential function, which imposes limitations that inductive voltage converters can avoid.

Charge pumps are often the best choice for powering an application requiring a combination of low power and low cost. The advantages of charge pump converters are that they do not require inductive elements, they are easy to design and have few components, and power dissipation is quite low as compared to other converter configurations. The disadvantages of charge pump configurations using switched-capacitor voltage converters for higher voltage conversions are the increased cost and space needed to accommodate large capacitors and the limited input-voltage range for practical operation.

A DC step-up (switching) voltage regulator combines inductive and capacitive step-up circuitry to produce high voltages while delivering low currents. Switching regulators operate by passing energy in discrete packets over a low-resistance switch, which they can step up, step down, and invert. A switching regulator can be practically operated over a wide input-voltage range and for high power requirements. However, they require magnetic design, and a higher component count, larger circuit area, and higher cost than charge pumps. Because of the need for increased power output, the devices employed a switching regulator to provide the voltage necessary for performing iontophoresis.

For the generation of the constant current, the device of the present invention utilizes an improved “Howland Charge Pump” configuration current regulator with an extended input voltage range (3-50V) and an adjustable output voltage range (0-60V). The circuit requires a high voltage input (provided by the switching regulator) and employs a precision voltage reference, an unregulated 5V to −5V voltage inverter, an operational amplifier, and few other resistive, capacitive, and inductive components. This type of regulator is widely used for voltage controlled current sources that have loads with one end (the patient) connected to ground.

Iontophoresis circuitry providing 40 μA at a high voltage 40VDC for sampling: To design the iontophoresis circuitry providing I_{L}=40 μA with the output voltage across the load Vₓ=40V and the load R_{L}=1MΩ, the Improved Howland Current Pump as shown is used.

Constraints required for this circuit for proper operation are:

R₂=R₄+R₅ (1)

R₁=R₃ (2)

Output Current, I_{L}=(V₁−V₂)R₂/(R₁R₅) (3)

Output Voltage across the load, Vₓ=(V₁−V₂)R_{L}R₂/(R₁R₅) (4)

Voltage on the inputs of the op-amp (common mode voltage), Vₐ=[V₁(R₂−R₅)+VₓR₁]/[R₁+(R₂−R₅)] (5)

Output of the Op-amp, Vₒ=[Vₐ(1+(R₂/R₁))]+[V₂(−R₂/R₁)] (6)

With R₁=1MΩ, R₂=100 kΩ, R₃=1MΩ, R₄=75 kΩ, R₅=25 kΩ, V₁=10V and V₂=0V,
Output Current, I_{L}=40 μA
Output Voltage across the load, Vₓ=40V
Voltage on the inputs of the op-amp (common mode voltage V_{cm}), Vₐ=37.91V
It satisfies the requirement of the op-amp OPA445 specifications 0V<V_{cm}<45V
Output of the Op-amp, Vₒ=41.7V
It satisfies the requirement of the op-amp OPA445 specifications 0V<Vₒ_{_{—}}ₘᵢₙ<45V.
   When load is changed to R_{L}=10 kΩ, keeping all other parameters same as shown.
Output Current, I_{L}=40 μA
Output Voltage across the load, Vₓ=0.4V
Voltage on the inputs of the op-amp (common mode voltage V_{cm}), Vₐ=1.07V
It satisfies the requirement of the op-amp OPA445 specifications 0V<V_{cm}<45V
Output of the Op-amp, Vₒ=1.18V
It satisfies the requirement of the op-amp OPA445 specifications 0V<Vₒ_{_{—}}ₘᵢₙ<45V

Testing at the limits showing that the current level is constant and that the circuit is in the functional range demonstrates that that the Improved Howland Current Pump circuit works over the anticipated load range for the sampling chamber (10 kΩ to 1MΩ).

Finally, patient protection circuitry shunts any excess current to circuit ground, eliminating any possibility that the circuitry can “shock” a patient and exceed FDA allowable current exposure. Using a resistor to monitor the current and a Zener diode to shunt current if it exceeds a threshold value allows the system to produce a current near the FDA maximum value, while discharging the current if it exceeds the recommended value.

Fabrication of miniaturized glucose sensor: The microsensor was fabricated as follows: a Teflon coated Pt wire (WPI, 0.125 mm i.d.) was cleanly cut and the surface of the cross-section of the wire was shown. An area of 0.0123 mm² of platinum wire cross-section was exposed with surrounding TFE coating. The area of exposed Pt wire is close to the proposed microelectrode area on the silicon electrode array chip.

Four different composition of glucose oxidase coating solutions were produced by mixing different weights of GOx, (1, 5, 10 and 20 mg) with 60 mg BSA and 0.1 mL PBS buffer (0.05M, pH 7.4) solution added to the membranes. Using gentle shaking to not damage the proteins, the enzyme and protein BSA solutions were dissolved. An 8 μL cross-linking reagent of glutaraldehyde (8% in water) was then mixed with each of above GOx-BSA solutions. After 5-8 minutes, the GOx-BSA-Glutaraldehyde mixture started to form a hydrogel. Four TFE-coated Pt wires were dipped ten times in this semi-gel solution. A thin gel film was observed on the tip of Pt surface. Then the gel-coated wires were air-dried and placed in refrigerator at 4° C. for 12 hours. Before usage, the electrode wires were immersed in PBS buffer solution for 5 minutes to provide sensor wetting.

Interference test for glucose sensors: The sensors, with the appropriate membranes and enzymes, were examined for an interference effect from molecules that affect the response of the electrode, either through direct electrode oxidation at the peak glucose oxidation potential thereby increasing the signal, reaction with the mediator thereby decreasing the glucose signal, or inhibition of the enzyme which also decreases the signal. Buffered test solutions with varying glucose concentrations and varying levels of interference molecules were produced and tested. The main interfering species for the glucose sensor is uric acid (UA, which has a typical plasma concentration of 0.2 mM). Glucose solutions, doped with uric acid, were measured using the electrodes.

A GOx coated Pt electrode was immersed in the PBS buffer solution (0.05M, pH 7.4) together with a Ag/AgCl reference electrode wire, and a counter electrode wire (Pt). The depth below the aqueous surface was 1.0 cm for all three electrodes. A stir bar was used to agitate the solution, rotating at a rate of 300 rpm. The glucose concentration was changed by adding concentrated glucose solution (1M) into the stirring buffer solution. Each addition of 25 μL of 1M glucose solution in 25 mL buffer solution increased the glucose concentration of the solution by 1 mM. The typical plasma range for glucose is 3-8 mM.

Based on the comparison of oxidatively derived current and concentration, and plotted, the glucose oxidase coated electrode exhibits a higher sensitivity (slope) to glucose (−0.038 μA/mM) than to uric acid (−0.027 μA/mM).

Assuming a normal plasma glucose level of 6 mM, the error introduced by the addition of a normal plasma concentration of uric acid (0.2 mM) is 2.96%. This interference can be reduced utilizing several different sensor methodologies. First, two sensors, one with the glucose oxidase membrane and the other with no membrane, can be utilized simultaneously and compared. Both respond equally to UA, however only the glucose oxidase membrane coated sensor responds to differing glucose concentrations. The second methodology is to coat the glucose oxidase membrane with a second, outer membrane in order to reduce the interfering effect. A thin protection membrane of Nafion (Nafion 117, Sigma Aldrich) can be used to eliminate the charged interfering substances, such as ascorbate and uric acid, etc.

Determine optimal glucose oxidase kinetics: All electrochemical measurements used a three-electrode system and were performed using a commercial potentiostat (CH-660). In these experiments, the sample solutions were tested with a stir-bar with a rotating rate of 600 rpm with a mini stir-bar (Teflon coated, 1 mm, i.d. and 10 mm length). A Pt counter electrode wire (0.25 mm i.d.) was immersed in the solution. The reference electrode Ag/AgCl wire (0.25 mm, i.d.) was also immersed in the solution. The sensing oxidation potential for the amperometric test was chosen at 0.3 mV, the oxidation potential of hydrogen peroxide versus an Ag/AgCl reference.

For measuring the amperometric i-t curve of enzyme-coated microelectrode, a series of solutions with different glucose concentrations (0, 4, 8, 12, 16, 20, 24 and 28 mM) were mixed used by spiking 100 mL concentrated 1M glucose in 2 mL 0.05 M PBS (pH 7.4) background solution. The figure shows an i-t curve resulting from the electrode coated with enzyme at a concentration of 1 mg GOx/60 mg BSA.

In studying the response kinetics, the relationship between enzyme concentration and response time helps to optimize the composition of coating hydrogel. In this test, current signal i reached its T₉₀ within 20 seconds (T₉₀ is the amount of time required for the sensor to reach 90% of the stabilized signal) when glucose concentration changes from 4 mM to 8 mM under 600 rpm stirring. It can also be seen that the dynamic concentration range of the microelectrode using 1 mg GOx/60 mg BSA composition is from 0 to ˜12 mM glucose.

The response time (T₉₀) is found to be affected by the geometry of the three electrode pattern. The distance between the working electrode, reference electrode and counter electrode, as well as the exposure area of reference and counter electrodes in solution influence the response performance.

With the increased concentration of enzyme in the membrane coating hydrogel, the response time (T₉₀) is reduced and the dynamic concentration range is extended.

Manufacture and test the multi-layer micro-fluidic conduits and chambers: In addition to the photo-patterning method of placing patterned conduits of the biocompatible PDMS materials onto the silicon substrate, AST is also optimizing screen-printing methodologies to pattern the PDMS micro-fluidic packaging materials onto the substrate. Two different biocompatible materials were examined for screen-printing: Dow Corning MDX-4210 and Sylgard 184. The Dow Corning MDX-4210 is a biomedical grade variation of Sylgard 184. The viscosity of these materials is similar to honey, which makes it difficult to remove the bubbles that are created when the two part material is thoroughly mixed.

AST's MSP-485 screen-printer was used to screen-print patterns of the MDX-4210 silicone to test the capabilities of this material and optimize the screen-printing parameters. This silicone was mixed at a ratio of 15:1 (base:catalyst) rather than the normal mixture of 10:1 to help lengthen the working time that the material was able to be utilized on the screen. To optimize the printing parameters, squeegee pressure and print speed were adjusted to provide a fully formed pattern with good leveling across the surface to ensure uniform patterns. The surface of the cured silicone was slightly non-uniform is thickness, thicker in some areas, thinner in others (variation of approximately 10 μm for a 100 μm thick membrane when the 15:1 mixing ratio was utilized). The optimal screen-printing parameters for this material are an offset of 50 mils and a pressure index setting of 50 and a print speed above 3 mm/sec.

Additionally, the MDX-4210, mixed at a 15:1, ratio was tested with the addition of 10 weight % of the Dow Corning 65 cst 360 silicone fluid to lower the viscosity of the material to be printed. The 360 silicone fluid does not get cross-linked into the polymer and is able to be washed out during the curing process. The printing with 10% 360 fluid demonstrated improved uniformity in thickness over the area of the print, with excellent definition of the material. The final thickness of this material was slightly thinner than the undiluted material leaving a membrane of 70 μm with immeasurable thickness differences across a 1.5 cm area.

Develop a reliable and reproducible thin film transfer technology: To improve the biocompatibility of the transdermal chemical sensing device, the three-dimensional (3D) layers of the micro-fluidic system were fabricated from an improved biocompatible material, PDMS MED10-6605 (Nusil), as opposed to GE-615 silicone rubber.

In general, there are two kinds of polymeric membranes required to fabricate the complex 3D micro-fluidic system, thin (1 to 10 μm) and thick (10 to 100+μm) patterned layers. PDMS is commonly used as a bulk (macro) material. The predominant fabrication process associated with PDMS is standard large feature molding.

The principle methodologies for the patterned thick PDMS film fabrication process are described as follows: (a) A photoresist layer (AZ 9260) is first deposited on the top of a solid substrate (e.g., glass or silicon, Table 2); (b) The photoresist layer is patterned by using conventional photo-lithography processes; (c) A PDMS pre-polymer solution (in the form of a viscous liquid) is poured over the substrate surface. Two different approached can be applied to remove excessive PDMS: (c1) a flat and smooth glass blade can be used to traverse the substrate surface while maintaining contact with the top surface of the photoresist layer; or (c2) a silicon/glass wafer can be placed on top of the wafer that contains the poured liquid PDMS, then a force is applied until the top wafer touches the top surface of the photoresist layer. Excessive PDMS pre-polymer is removed, leaving PDMS only in recessed regions between protruding photoresist molds; (d) After the remaining PDMS is thermally cured, Reactive Ion Etching (Plasma) is used to remove the excess, thin layer of PDMS on the top surface of the photoresist; and (e) The photoresist mold is removed selectively by using PRS2000 instead of acetone, which is absorbed into PDMS material causing swelling and delamination of the PDMS material. The height of the resultant PDMS pattern corresponds very accurately to the thickness of the photoresist. Both fabrication methodologies have been employed and have demonstrated very good results: (i) very strong adhesion of PDMS onto the substrates; (ii) well controlled lateral dimensions and heights (the 12 μm features on the mask ended up 18 μm wide at the top and 11 μm wide at the bottom of a 50 μm tall patterned PDMS layer). The figure shows a photograph of completed 50 μm deep PDMS actuator water containers.

TABLE 2
Thick PDMS film patterning process

| | | | Target | |
|---|---|---|---|---|
| Step | Process/Furnace | Specifications | Data | Comments |
| 1 | SI wafer | P type, <100> | 1 wafer | |
| 2 | Hard-bake | 110° C., | | |
| | | 60 mins | | |
| 3 | HMDS | spread @ 500 | | ≧4K is highly |
| | | rpm for 10 | | recommended |
| | | seconds, and | | |
| | | spin @ 4K rpm | | |
| | | for 20 seconds | | |
| 4 | AZ 9260 | spread @ 500 | 25 μm | Sit for |
| | | rpm for 10 | | 5 mins |
| | | seconds, and | | |
| | | spin @ 900 rpm | | |
| | | for 20 seconds | | |
| 5 | Soft-bake | 90° C., | | |
| | | 14 minutes | | |
| 6 | Bead removal | Spin @ 1K | | Swab with |
| | | rpm, 60 second | | Acetone |
| | | (first 20 sec.) | | |
| 7 | AZ 9260 | spread @ 500 | Total | Sit for |
| | | rpm for 10 | 50 μm | 5 mins |
| | | seconds, and | | |
| | | spin @ 900 rpm | | |
| | | for 20 seconds | | |
| 8 | Hard-bake | 90° C., | | |
| | | 30 minutes | | |
| 9 | Photo-lithography | | | |
| 10 | Development | AZ400K:water (1:3) | | Observing the |
| | | 5 minutes with | | pattern and |
| | | agitation | | make sure no |
| | | | | residual left |
| 11 | Dektek | | 50 μm | |

Manufacture and test the multi-layer micro-fluidic conduits and chambers: Two different biocompatible materials were examined during screen-printing: Dow Corning MDX-4210 and Sylgard 184. The Dow Corning MDX-4210 is a biomedical grade variation of Sylgard 184. The viscosity of these materials is similar to honey, which makes it difficult to remove the bubbles that are created when the two part material is thoroughly mixed.

An MSP-485 screen-printer was used to screen-print patterns of the MDX-4210 silicone to test the capabilities of this material and optimize the screen-printing parameters. This silicone was mixed at a ratio of 15:1 (base:catalyst) rather than the normal mixture of 10:1 to help lengthen the working time of the material. To optimize the printing parameters, squeegee pressure and print speed were adjusted to provide a fully formed pattern with good leveling across the surface to ensure uniform patterns. The surface of the cured silicone was slightly non-uniform in thickness, thicker in some areas, thinner in others (variation of approximately 10 μm for a 100 μm thick membrane when the 15:1 mixing ratio was utilized). The optimal screen-printing parameters for this material are an offset of 50 mils and a pressure index setting of 50 and a print speed above 3 mm/sec.

The initial studies utilizing MDX-4120 resulted in a bead forming at the edge of the print. A bead is a thicker region of material usually at the edge of the pattern typically caused by surface tension. The printing parameters were altered to alleviate this problem; however the bead problem still existed. To resolve the problem of an uneven surface during screen-printing, a different silicone material was tested. The material was LSR 4340 from Rhodia. It has a high percent elongation and low adhesion to glass and other molding substrates, which makes it desirable for producing the patterned thin films that can subsequently be transferred to another substrate. The LSR material is very viscous, so it was diluted with hexamethyldisiloxane to remove air bubbles that resulted from mixing, and improve the surface leveling. The material printed evenly on a glass substrate without a bead at the edge; however, the surface of the material had a grainy texture due to the high thixotropy, and was much thinner than expected. It is hypothesized that, as the screen snapped off of the surface, part of the printed membrane was removed from the surface because it was still attached to the screen. Both hard and soft durometer squeegees were examined to alleviate this issue without noticeable differences. Additionally, changing the squeegee pressure and print speeds did not yield optimal operating conditions.

Additionally, the MDX-4210, mixed at a 15:1, ratio was tested with the addition of 10 weight % of Dow Corning 65 cst 360 silicone fluid to lower the viscosity of the material to be printed. The 360 silicone fluid does not get cross-linked into the polymer and is able to be washed out during the curing process. Printing with 10% 360 fluid demonstrated improved uniformity in thickness over the area of the print, with excellent definition of the material. The final thickness of this material was slightly thinner than the undiluted material leaving a membrane of 70 μm with immeasurable thickness differences across a 1.5 cm area.

Laser machining of glass can also be done. Researchers at the University of Michigan have been able to produce holes and channels smaller then a micron in size. As an alternative, micro-machined plastic is a good alternative to make inexpensive complicated micro-fluidic devices. Several different types of plastic samples were obtained for testing and machining including: Noryl, Lexan, Xylex, Ultem, and acrylic. The acrylic plastic had the highest contact angle of 70 degrees, while Lexan had the lowest of 64 degrees. The Noryl, Xylex, and Ultem had a contact angle of 65 degrees, meaning they are all hydrophilic to varying degrees.

Based upon the previously reported fabrication methodologies for (a) PDMS thick film (greater than 50 μm) patterning and (b) PDMS thin film transfer utilizing flexible polymeric substrate, the pump production process and fabricated micro-fluidic pumps/valves which are crucial and compatible with the production of the three-dimensional micro-fluidic glucose sampling and analysis micro-system have been modified. First, the micro-heaters were fabricated on a silicon wafer on top of a MEMS based thick silicon oxide (50 μm) fabrication technology, which acts as the thermal isolation layer and was developed previously. Second, about 50 μm thick PDMS (Dow Corning Sylgard 184) is patterned and cured to form a water container. Finally, a water drop is deposited into the water container and a cured PDMS film (Nusil MED10-6605), with an approximate thickness of 25 μm, is bonded on top of PDMS water container to physically seal the water into the container.

The mechanism of pumping and valving can be explained in the following way: the membrane is actuated (popped-up) by vaporizing water, which expands and forces the thin PDMS membrane to actuate. This occurs when electrical power is applied to the micro-heaters. As the membrane actuates, it occludes a conduit and solution can be forced in a particular direction. The figure shows optical microscopic pictures of the comparison between the un-actuated (left) and actuated membrane. The input voltage is 15 volts, and actuation frequency is 25 Hz.

Use various techniques for sampling interstitial fluid to collect the glucose: The prototype sampling system contains integrated electrical connections to the sampling chamber to allow application of the various electro-motive techniques to obtain interstitial fluid samples. To complete the circuit for iontophoretic sampling, a second electrode connection is placed on the skin with a small amount of colloidion paste to complete the electrical circuit. By manipulating the buffering solution, the current, and the voltage applied to the system, iontophoresis, electro-osmosis, and electroporation is employed, tested, and optimized.

Osmotic methods take advantage of concentration gradients to draw small, lipophilic ions across the skin barrier. In humans, the stratum corneum is negatively charged and, therefore, allows cationic particles to diffuse across the barrier at a much higher rate than anionic particles. Often, salt or sugar solutions are utilized to provide the osmotic gradient to draw the interstitial fluids from the body.

Electro-osmosis is a process by which an externally applied potential is used to mobilize cations such as sodium, which freely cross the stratum corneum, to transfer their momentum to neutral molecules around them. This technique has been used to measure glucose, non-invasively, utilizing large electrodes and transdermal patches with excessively large surface areas and volumes.

Electroporation uses short (100-300 ms) pulses of very high voltage (50-250V) to increase transdermal interstitial fluid transport. While this method increases mass transport across the dermal membrane by several orders of magnitude, there are certain disadvantages: the high voltage required is incompatible with standard CMOS circuitry; the high voltage pulses can be irritating to the patient; and the transport may not be fully reversible.

A small circuit was designed utilizing off-the-shelf parts to deliver the currents necessary for the system. This is necessary in order to protect the subject, and deliver a specific and uniform current. The circuit is powered by batteries to assure safety. Research has shown that when applying electrical current, the resistance of the skin and flow of molecules changes significantly over the first hour. The circuitry operates under closed-loop feedback control to account for changes in current flow (and interstitial fluid transport) over time.

To ensure patient safety, batteries power the circuit. Special consideration was made to insure that the patient is protected from potentially dangerous current exposure. These considerations include failure-mode analysis, i.e. in the case that an electrode becomes disconnected, or electrical resistance increases due to electrode fouling. Current limiting circuitry was employed to ensure that, even in failure mode, dangerous currents are not injected into the body and that the path for current conduction never leaves the upper layers of the skin.

Detecting Glucose: There are several methods for determining the level of glucose in biological solutions. Of these, amperometric measurement of the byproducts from a glucose oxidase catalyzed reaction of glucose to reduced glucose and hydrogen peroxide was examined and compared with finger-prick blood glucose determinations. Amperometric detection was chosen due to its low detection limits, and sensor fabrication techniques that are amenable to ultra-miniaturization.

Requirements for the glucose assay system are based upon the physiological range of glucose present in blood (and interstitial fluid, which are in equilibrium with blood concentrations). Euglycemic levels fall in the range of 75 to 165 mg/dl, varying from person to person, according to age and physical factors. Blood glucose levels below 75 mg/dl are considered hypoglycemic and above 165 mg/dl are considered hyperglycemic. The device of the present invention is able to monitor glucose concentrations between 0 mg/dl and 300 mg/dl as shown in the standard curve in section F.

Calibration factors were established in order to correlate glucose determinations obtained transdermally from interstitial fluid with actual blood glucose values. The calibration factors include compensation for the decrease in glucose concentration in interstitial fluid, which is in equilibrium with blood glucose concentrations, as well as compensation for the decrease in glucose concentration due to the extraction of interstitial fluid through skin. This calibration was affected both from modeling parameters, as well as from actual empirical measurements, i.e. comparing finger-prick blood glucose determinations with transdermally obtained interstitial fluid glucose determinations. In this manner, each individual can self-calibrate the agent delivery system upon initial application. This technique improves accuracy by allowing compensation for different skin types and different locations of patch application.

Using serial dilution of glucose samples in phosphate buffered saline solution; fluid samples containing varying concentrations of glucose were produced. Standard calibration curves were generated and compared for accuracy and precision. The results from these tests were compared with standard glucose assays (Sigma Chemical Corp.).

Miniaturization of glucose sensors: The system of the present invention utilizes miniaturized, amperometric sensors, coated with a membrane containing glucose oxidase, to transduce the concentration of glucose within the interstitial fluid samples. There are two reasons to utilize these microscopic sensors. The first, most obvious reason involves the fact that the interstitial fluid is presented in extremely small volumes, hence the requirement for small sensors. The second is less obvious. As opposed to potentiometric sensors, whose functionality and lifetime decreases as the size of the membrane (and therefore the contained ionophore) decreases. With amperometric sensors, a charge is placed upon the sensors, and a period of time is required for the dipole molecules in the surrounding hydration shell to align with the electronic field. As the size of the sensor decreases, the size of the hydration shell decreases, hence decreasing the amount of time required for the dipole realignment. This not only increases the maximum sampling rate, but also increases the sensitivity and signal to noise ratio by a substantial amount.

Further, the utilization of microscopic sensors provides other advantages. First, the microscopic sensors are produced utilizing solid state silicon manufacture techniques. These techniques allow for inexpensive mass production, with exacting specifications, not only within a single manufacture run, but from year to year. Second, the utilization of a microscopic screen printer provides for economic production of the specialized enzymatic membrane coated sensors due to the automation provided by this device.

### Examples

### Category 3

The agent delivery system may also be adapted for outpatient and “in-office” non-surgical cosmetic procedures. The present invention would eliminate the use of needles and increase the surface area treated. The agent delivery system may be programmed to deliver cosmetic treatment agents generally injected under the skin via pulsatile administration.

The examples provided for this agent delivery system category involves: a pulsatile delivery device, an automated controller to provide programmed pulsed delivery, agent:polymer matrix, a biocompatible membrane and adhesive to attach the delivery reservoir to the skin. A feedback unit to sample the patient utilizing a reverse iontophoretic method. Reagent, reaction and waste chambers and/or reservoirs and a microfluidic system to transport the fluids between reservoirs. The unit has an integrated USB port and may be adapted for wireless signal transmission.

The present invention for this category may be utilized, but not limited to administering: collagen pre-cursors, Botox™, wound healing agents and may also be utilized to provide an electromagnetic field to stimulate tissue repair. These examples are for illustrative purposes and intended to be descriptive rather than limitations.

### Example 1

### SCITS

A Sensor-fitted Cosmetic Improvement Transdermal System (SCITS) capable of directing the deposition of collagen precursor molecules and actively directing their alignment, in a non-invasive manner, such that wrinkles can be removed and plasticity can be returned to the skin. The proposed non-invasive transdermal SCITS is able to self monitor the progress by measure epithelial-derived currents from sodium-potassium (Na⁺−K⁺) pumps in the plasma layer membrane of basal layer keratinocytes, and the dipole alignment of the collagen precursors, the zeta potentials.

The goals also include the development and incorporation of custom electrode systems to provide various modes of electro-magnetic stimulation to the face in the attempt to target and induce the formation of collagen, in the appropriate orientation, at a high rate of deposition, in a non-invasive manner. Three different methods of stimulation can be used: direct electrical stimulation, capacitive coupling, and oscillating magnetic fields generated by an induction coil.

The transdermal system includes a chamber for containing various pre-cursor substrates. Additionally, the transdermal system of the present invention includes electrode systems or devices to provide various modes of electromagnetic stimulation. The transdermal system of the present invention can be utilized to target and induce the formation of collagen, in the appropriate orientation and at a high rate of deposition, in a non-invasive manner. As a result, the skin's elasticity and plasticity can be improved and/or restored.

The present invention is capable of laying a scaffold of precursor substrates in an individual. The scaffold can be established in the epidermis, dermis, subcutaneous fat, or in any other layer within the body of an individual. The scaffold is defined as a supporting framework of precursor substrates wherein the precursor substrates are aligned and/or oriented in a manner that aids in the formation of collagen. Alignment and/or orientation of precursor substrates occur via electromagnetic stimulation. The electromagnetic stimulation increases the growth rate and control of orientation of the newly formed collagen molecules.

The basis of this embodiment of the present invention depends upon existence of basement membranes of the skin. Basement membranes, found in most tissues of biological organisms, are thin layers of specialized extracellular matrix that form supporting structures on which epithelial cells grow. Basement membranes can act as scaffolds, providing structural cues as well as enabling nutrition by diffusion until grafting occurs. They are in close apposition to the cells, provide mechanical support, divide tissues into compartments, and influence cellular behavior. Basement membranes are molecular composites of collagen, proteoglycans, and noncollagenous glycoproteins. Collagen is the major constituent of biological basement membranes and provides a scaffold for other structural macromolecules by forming a network via molecular interactions. The composite network is formed by a self-assembly process leading to a relatively regular structure. The resulting scaffold contains binding sites for cells. The nature and number of binding sites, and the way they are presented, are detected by cell surface receptors and affect cell growth.

The present invention can be used in a variety of settings and on a variety of skin surfaces. The present invention can be adapted to be any size or shape as desired. For example, the system of the present invention can be the size and length of a typical wrinkle on the face. Alternatively, the system can cover the entire face of an individual. Moreover, the present invention can be a single unit or composed of various components. Parts of the system of the present invention can also be disposable or reusable, depending upon the desired application.

Fabrication of the system of the present invention is based upon the development of a process flow. The fabrication process utilizes bulk silicon micro-machining techniques to produce the isolation windows, and thick film screen-printing techniques, spin coating, mass dispensing, or mechanical dispensing of actuation membranes.

The present invention has numerous embodiments. In one embodiment, there is provided a biochamber transdermal system including at least one perfusion chamber for containing pre-cursor substrates. Further, the system includes an electrical field-stimulating device for aligning the pre-cursor substrates. Optionally, the system can include a sensor device for measuring a zeta potential.

The perfusion chamber of the present invention is any structure capable of containing pre-cursor substrates. The chamber can be, but is not limited to, any type of tube, pipe, planar channel, conduit, or any other similar chamber known to those of skill in the art. The chamber can be made of numerous materials known to those of skill in the art. Examples of such materials include, but are not limited to, silicon, plastic, glass, polymers, translucent acrylic plastic cast sheeting, combinations thereof, and any other similar materials known to those of skill in the art.

As described above, the perfusion chamber contains pre-cursor substrates. These pre-cursor substrates are pre-cursor molecules, compounds, and/or materials capable of being absorbed by the skin to be converted by the body into a collagen scaffold. The pre-cursor substrates form the basis of a collagen scaffold in the skin of an individual. The collagen scaffold increases the plasticity and elasticity of the skin. Further, the scaffold can improve the appearance of the skin. Examples of pre-cursor substrates that can be utilized with the present invention include, but are not limited to, porous, cross linked collagen-glycosaminoglycan, polytetrafluoroethylene, poly-L-lactide and poly(ethyleneoxide)-poly(butyleneterephthalate), polyglactin, polyglycolic acid, biosynthetic materials, hydrocolloid-like material, and any other similar pre-cursor substrates known to those of skill in the art.

The biochamber transdermal system of the present invention includes an electrical field-stimulating device for aligning the pre-cursor substrates. By aligning or orienting the pre-cursor substrates, a collagen scaffold can be formed under the skin of an individual.

The application of external electric fields to tissue has been shown to have a significant effect on healing in animal models and clinical trials. These effects include increases in the concentration of adenosine triphosphate and rate of amino acid uptake, decrease in tissue oxygen tension, and increase in fibroblast proliferation and collagen production. Electrical stimulation can also aid wound healing indirectly by orienting collagen.

The electrical stimulating device can produce numerous types of electrical fields including, but not limited to, direct electrical stimulation, capacitive coupling, oscillating magnetic, combinations thereof, and any other similar electrical fields known to those of skill in the art

Direct current stimulation can be achieved by using platinum electrodes applied on the skin to generate a local electric field. For direct electrical stimulation, a potential can be applied between two platinum electrodes located on either side of the perfusion chamber, causing ionic and electronic current to flow between the electrodes. The voltage applied can be kept below 1.5 volts to prevent electrolysis of water. Currents between nanoamperes and milliamperes can be employed and in accordance with standards well known to those of skill in the art.

Capacitive coupling of an electric field can be generated with two oppositely charged plate electrodes. With this method, it is necessary to use high frequencies to generate a sufficient current flow. For capacitive coupling, 50 volt, 0.5 Hz bipolar square waves can be produced by the electrodes.

Oscillating magnetic field can be generated by an induction coil. The varying magnetic field can generate an electric field that is proportional to the rate of change of the magnetic field. A magnetic field that varies with time can generate an electric field that is proportional to the rate of change of the magnetic field.

Optionally, the system of the present invention can include a sensor device of the present invention is used to measure surface electrical properties in the skin. By measuring the surface electrical properties (e.g., zeta potentials), progress of the formation of the collagen scaffolding can be monitored. The zeta potential can serve as an indicator of biomimetic graft efficacy. Additionally, evaluating epithelial derived zeta potential has a direct correlation to early adherence properties and cell growth. The zeta potential is related to the net surface charge of the tissue preparation: a positive correlation exists between tissue adherence properties and zeta potential. The zeta potential can be calculated from the streaming potential using the Helmholtz equation:

Z=4πηKVₛ/DP

where Z=zeta potential in millivolts

η=viscosity in poise of test fluid

K=specific conductance of fluid in stathmos per centimeter

Vₛ=streaming potential measured across electrodes in millivolts

D=dielectric constant of fluid

P=pressure difference between measuring electrodes in dynes per square centimeter

Zeta potentials originate from epithelial-derived currents created by sodium-potassium (Na⁺—K⁺) pump in the plasma layer membrane of basal layer keratinocytes. According to the Burr et al. reference, positive epithelial electrical potentials (TEP) exist in abdominal skin. Electrical potentials of skin wounds changed in polarity during the first few days of healing as the number of cells increased, suggesting a possible connection between this shift in potential and the healing process. This observation, in addition to others, provides increasing evidence that electric and magnetic fields are able to alter the process of mammalian soft-tissue repair.

The sensor device is at least one electrode. The electrode can be made of numerous materials including, but is not limited to, polysilicon, elemental metal, silicide, metals, platinum, silver wire, combinations thereof, and any other similar material known to those of skill in the art. For example, the electrode can be prepared from fine silver wire electrolytically chlorided in a hydrochloric acid solution. The electrode can be integrated into the system of the present invention on either side of the perfusion chamber. The potential existing between the electrodes can be amplified by a high-impedance instrumental amplifier. The potential can then be monitored and stored using a computerized analog to digital converting system. Those of skill in the art can manufacture and connect the sensor device to the biochamber transdermal system of the present invention.

In another embodiment of the present invention, there is provided a system mask for placement on the entirety of an individual's face. The system mask includes a disposable matrix having pre-cursor substrates situated therein. The disposable matrix can cover a portion or the entire face. The disposable matrix can be made of numerous materials including, but not limited to, polymers, fabric, cloth, solid gel-like material, and any other similar materials known to those of skill in the art.

As set forth above, examples of pre-cursor substrates that can be utilized with the present invention include, but are not limited to, porous, cross linked collagen-glycosaminoglycan, polytetrafluoroethylene, poly-L-lactide and poly(ethyleneoxide)-poly(butyleneterephthalate), polyglactin, polyglycolic acid, biosynthetic materials, hydrocolloid-like material, and any other similar pre-cursor substrates known to those of skill in the art. These pre-cursor substrates can be deposited on or within the matrix utilizing methods well known to those of skill in the art.

The system mask also includes a base structure that is releasably attached to the matrix of the system. The base structure is a mask-like structure made of materials including, but not limited to, metal, plastics, polymers, conductive materials, non-conductive materials, and any other similar materials known to those of skill in the art. Electrical field stimulating devices are operatively attached to the base structure for aligning and/or orienting the pre-cursor substrates into a collagen scaffold. The electrical field stimulating devices can be attached a portion of the base structure. If conductive materials are used for the base structure, then the electrical field ca be applied across the entirety of the matrix of the system. Alternatively, if non-conductive materials are used, various electrical field stimulating devices can be used to specifically target certain areas of the face so that pre-cursor alignment and/or orientation occurs in specified areas.

As set forth above, the electrical stimulating device can produce numerous types of electrical fields including, but not limited to, direct electrical stimulation, capacitive coupling, oscillating magnetic, combinations thereof, and any other similar electrical fields known to those of skill in the art. Production of these electrical fields can be achieved using devices including, but not limited to, various electrodes, induction coils, and other similar electrical field producing devices known to those of skill in the art.

Finally, the present invention provides for various methods. One method includes a method of aligning and/or orienting collagen pre-cursor substrates by applying collagen pre-cursors to the skin surface of an individual; stimulating the collagen pre-cursors with electrical fields selected from the group consisting of direct electrical stimulation, capacitive coupling, oscillating magnetic, and combinations thereof; and aligning the collagen pre-cursors.

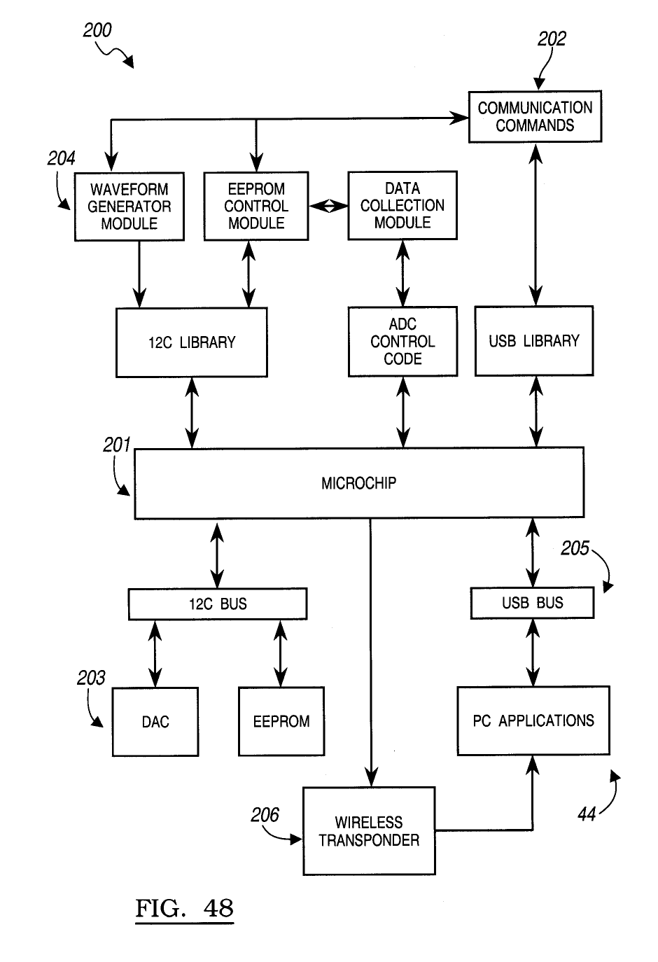

FIG. 48 illustrates a software interface block diagram 200. The microcontroller (Microchip) 201 controls all functions for the agent delivery device circuit to deliver controlled electrical current to the electrodes and allows programmability for the waveform parameters 204. Equipped with an on-board timer, the microcontroller will be able to deliver time-based current waveforms to the electrodes by use of a digital-to-analog converter (DAC) 203 and a constant-current analog circuit. This will ensure that the same amount of current is delivered over a range of skin impedances. These time parameters (period, duty cycle, diminishment rate) can be programmed to the microcontroller using the USB bus 205 from a PC 44. Alternatively, the wireless transponder 206 may be used instead of the USB bus 205. In addition, this prototype device is equipped with an analog-to-digital converter (ADC) and non-volatile memory 204 which will be used to record voltage and current delivered over a period of time in order to verify proper functionality.

Throughout this application, author and year and patents by number reference various publications, including United States patents. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A method for delivering an agent to a patient from an agent delivery device, the agent delivery device including:
an agent delivery reservoir containing an agent to be administered to a patient;
an electrolyte that is mixed with the agent and contained in the reservoir and traps the agent until electric current is applied;
an agent delivery surface in communication with the electrolyte-agent mixture, the agent delivery surface adapted to contact the patient and deliver agent received from the reservoir to the patient; and
a controller in communication with the electrolyte-agent mixture, the controller providing a series of control pulses to the electrolyte-agent mixture, each pulse allowing the delivery system to administer a portion of the agent to the patient, the series of pulses providing a temporally varying concentration of agent in the patient,
the method comprising:
contacting the patient with the agent delivery surface; and
operating the controller to administer the agent to the patent.

2. The method of claim 1, wherein the electrolyte comprises an iontophoretic electrically conductive material.

3. The method of claim 1, wherein the agent comprises: an anti-malarial agent.

4. The method of claim 1, wherein the agent comprises an antimalarial drug.

5. The method of claim 4, wherein the antimalarial drug is selected from the group consisting of amodiaquine, artemether, artemisinin, artesunate, atovaquone, cinchonine, cinchonidine, chloroquine, doxycycline, halofantrine, mefloquine, primaquine, pyrimethamine, quinine, quinidine, sulfadoxine, and combinations thereof.

6. The method of claim 1, wherein the agent comprises a hormone.

7. The method of claim 6 wherein the hormone is selected from the group consisting of gonadotropin releasing hormones (GnRH), estradiol, progesterone, growth hormone, thyroid stimulating hormone (TSH) prolactin, human parathyroid hormone buserelin, insulin, and combinations thereof.

8. The method of claim 1, wherein the agent comprises an antiretroviral drug.

9. The method of claim 8 wherein the antiretroviral drug is selected from the group consisting of abacavir, didanosine, indinavir, lamivudine, nevirapine, ritonavir, saquinavir mesylate, zalcitabine, zidovudine, and combinations thereof.

10. The method of claim 1, wherein the agent comprises an antibiotic drug.

11. The method of claim 1 wherein the antibiotic drug is selected from the group consisting of ampicillin, azithromycin, doxycycline, erythromycin, penicillin, tetracycline, and combinations thereof.

12. The method of claim 1, wherein the agent comprises an antipsychotic drug.

13. The method of claim 1, wherein the agent comprises an addictive agent.

14. The method of claim 13 wherein the addictive agent is selected from the group consisting of nicotine, morphine, methadone, oxycontin, cocaine, barbiturates and combinations thereof.

15. The method of claim 1, wherein the agent comprises a chemotherapeutic cancer agent.

16. The method of claim 15 wherein the chemotherapeutic cancer agent is selected from the group consisting of Buserelin, Taxol, and combinations thereof.

17. The method of claim 1 wherein the agent comprises a cosmetic anti-wrinkle agent.

18. The method of claim 17, wherein the cosmetic anti-wrinkle agent is selected from the group consisting of acollagen, collagen-glycosaminoglycan, polytetrafluoroethylene, poly-L-lactide and poly(ethyleneoxide)-poly(butyleneterephthalate), polyglactin, polyglycolic acid, biosynthetic materials, hydrocolloid-like materials, and combinations thereof.

19. The method of claim 1, wherein the agent comprises a naturally occurring or synthetic hydrophilic or hydrophobic agent.

20. The method of claim 1, wherein the agent comprises an analgesic.

21. The method of claim 20, wherein the analgesic agent is selected from the group consisting of non-steroidal anti-inflammatory drugs, steroids, COX-1 inhibitors, COX-2 inhibitors, and combinations thereof.
